(19) United States
(12) Patent Application Publication
(10) Pub. No.: US 2015/0272980 A1
(43) Pub. Date: Oct. 1, 2015

(54) Dosing and Administration of Oligonucleotide Cancer Therapies

(71) Applicant: PRONAI THERAPEUTICS, INC., Plymouth, MI (US)

(72) Inventors: Wendi Veloso Rodrigueza, Boston, MA (US); Mina Patel Sooch, West Bloomfield, MI (US); Shari Kay Gaylor, Kalamazoo, MI (US); Richard Adam Messman, Brighton, MI (US); Michael James Woolliscroft, Ann Arbor, MI (US)

(21) Appl. No.: 14/440,867

(22) PCT Filed: Nov. 5, 2013
(86) PCT No.: PCT/US2013/068516

§ 371 Date: May 5, 2015

Related U.S. Application Data
(60) Provisional application No. 61/722,526, filed on Nov. 5, 2012.

(51) Int. Cl. A61K 31/7088; A61K 45/06; A61K 9/127

(52) U.S. Cl. CPC A61K 31/7088; A61K 9/127; A61K 45/06

(57) ABSTRACT

The present invention relates to cancer therapies, compositions, and methods of using the same. In particular, the present invention provides methods of dosing and administration of cancer therapies comprising the administration of oligomers and liposome formulations of oligomers, wherein the cancer is mediated by the bcl-2 oncogene. In some aspects, the oligomers or liposome formulations of oligomers are administered in combination with one or more other therapeutic agents.

### TECHNICAL FIELD OF THE INVENTION

The present invention relates to cancer therapies, compositions, and methods of using the same. In particular, the present invention provides methods of dosing and administration of cancer therapies comprising the administration of oligomers and liposome formulations of oligomers, wherein the cancer is mediated by the bcl-2 oncogene. In some aspects, the oligomers or liposome formulations of oligomers are administered in combination with one or more other therapeutic agents.

### PRIORITY CLAIM

This application claims priority to U.S. Application Ser. No. 61/722,526, filed Nov. 5, 2012. The entire contents of the aforementioned application are incorporated herein by reference.

### SEQUENCE LISTING

This application incorporates by reference in its entirety the Sequence Listing entitled “Sequence_{—}2012. txt” (698 KB) which was created Nov. 5, 2012 and filed herewith on Nov. 5, 2012.

### BACKGROUND OF THE INVENTION

Cancer survival rates vary depending on the cancer site/type with overall survival rates for all types being ˜50%. Tremendous advances have been made treating patients with chemotherapeutic and target therapeutic drugs, as cocktails or combinations. In addition, genetic screening and phenotyping cell types for markers and their response to therapy have greatly increased survival rates. Despite these multi-attack approaches, cancer death rates increase yearly. It is clear that most major-incidence metastatic cancers fail to respond, or in some cases, respond initially to therapy, but then fail to respond due to drug resistance resulting from the activation of alternative survival pathways. Patients succumb to the disease due to complications that arise from the primary tumor and/or metastases. Clearly, these high mortality rates suggest a need for additional therapeutic agents that complement and enhance the armament against cancer.

Oncogenes have become the central concept in understanding cancer biology and may provide valuable targets for therapeutic drugs. In many types of human tumors, including lymphomas and leukemias, oncogenes are overexpressed, and may be associated with tumorigenicity (Tsujimoto, et al., Science 228:1440-1443 (1985)). For instance, high levels of expression of the human bcl-2 gene have been found in all lymphomas with a t(14; 18) chromosomal translocations including most follicular B cell lymphomas and many large cell non-Hodgkin's lymphomas. High levels of bcl-2 gene expression have also been found in certain leukemias that do not have a t(14; 18) chromosomal rearrangement, including most cases of chronic lymphocytic leukemia acute, many lymphocytic leukemias of the pre-B cell type, neuroblastomas, nasopharyngeal carcinomas, and many adenocarcinomas of the prostate, breast and colon. (Reed et al., Cancer Res. 51:6529 [1991]; Yunis et al., New England J. Med. 320:1047; Campos et al., Blood 81:3091-3096 [1993]; McDonnell et al., Cancer Res. 52:6940-6944 [1992); Lu et al., Int. J Cancer 53:29-35 [1993]; Bonner et al., Lab Invest. 68:43A [1993]. Other common oncogenes include TGF-α, c-ki-ras, ras, Her-2 and c-myc.

Gene expression, including oncogene expression, can be inhibited by molecules that interfere with promoter function. Accordingly, the expression of oncogenes may be inhibited by single-stranded oligonucleotides.

### SUMMARY OF THE INVENTION

Some aspects of the invention comprise a method of treating cancer, comprising administering to a patient an effective amount of an oligonucleotide compound comprising an oligomer that hybridizes under physiological conditions to an oligonucleotide sequence selected from SEQ ID NO:1249 or SEQ ID NO:1254 or the complements thereof, wherein the oligonucleotide is administered on one or more days of a dosing cycle.

In some aspects, the oligomer may be administered in a liposome formulation. In some aspects, the liposome formulation is an amphoteric liposome formulation. In some aspects, the amphoteric liposome formulation may comprise one or more amphoteric lipids, which may be formed from a lipid phase comprising a mixture of lipid components with amphoteric properties.

In some aspects, the mixture of lipid components may be selected from the group consisting of (i) a stable cationic lipid and a chargeable anionic lipid, (ii) a chargeable cationic lipid and chargeable anionic lipid and (iii) a stable anionic lipid and a chargeable cationic lipid. In some aspects, the lipid components may comprise one or more anionic lipids selected from the group consisting of DOGSucc, POGSucc, DMGSucc, DPGSucc, DGSucc, DMPS, DPPS, DOPS, POPS, DMPG, DPPG, DOPG, POPG, DMPA, DPPA, DOPA, POPA, CHEMS and Cet-P. In some aspects, the lipid components may comprise one or more cationic lipids selected from the group consisting of DMTAP, DPTAP, DOTAP, DC-Chol, MoChol, HisChol, DPIM, CHIM, DORIE, DDAB, DAC-Chol, TC-Chol, DOTMA, DOGS, (C18)2Gly+ N,N-dioctadecylamido-glycine, CTAP, CPyC, DODAP and DOEPC.

In some aspects, the lipid phase further comprises neutral lipids, which may be selected from sterols and derivatives thereof, neutral phospholipids, and combinations thereof. The neutral phospholipids may be phosphatidylcholines, sphingomyelins, phosphoethanolamines, or mixtures thereof. The phosphatidylcholines may be selected from the group consisting of POPC, OPPC, natural or hydrogenated soy bean PC, natural or hydrogenated egg PC, DMPC, DPPC or DOPC and derivatives thereof and the phosphatidylethanolamines selected from the group consisting of DOPE, DMPE, DPPE and derivatives thereof.

In some aspects, the amphoteric liposomes comprise DOPE, POPC, CHEMS and MoChol. In some aspects, the molar ratio of POPC/DOPE/MoChol/CHEMS is about 6/24/47/23.

In some aspects of the present method, the oligomer hybridizes under physiological conditions to the oligonucleotide sequence SEQ ID NO:1249 or the complement thereof. In some aspects, the oligomer may comprise an oligomer selected from the group consisting of SEQ ID NOs:1250, 1251, 1252, 1253, 1267-1477 or the complements thereof. In some aspects, the oligomer may comprise an oligomer selected from the group consisting of SEQ ID NOs:1250, 1251, 1289-1358 or the complements thereof. In some aspects, the oligomer may comprise SEQ ID NO:1250 or 1251.

In some aspects, the method may further comprise administering an additional chemotherapeutic agent or in conjunction with immunotherapy, radiotherapy or surgical therapy. The additional chemotherapeutic agent, immunotherapy, radiotherapy or surgery may be administered before, simultaneous with, or after the administration of the oligonucleotide compound of claim 1. In some aspects, the additional chemotherapeutic agent may be selected from alkylating agents (e.g., nitrogen mustards, nitrosoureas, tetrazines, aziridines, cisplatins), anti-metabolites (e.g., anti-folates), anti-microtubule agents (e.g., paclitaxel, vinca alkaloids), topoisomerase inhibitors (e.g., irinotecan, topotecan), cytotoxic antibiotics (e.g., doxorubicin, daunorubicin), metformin, insulin, 2-deoxyglucose, sulfonylureas, anti-diabetic agents generally, mitochondrial oxidative-phoshorylation uncoupling agents, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), mTOR inhibitors, or agents that influence cancer metabolism. In some aspects, the additional agent may be a targeted agent involved in blocking pathways involved tumor suppression, genesis, progression, growth, proliferation, migration, cell cycle, cell signaling, metastases, invasion, transformation, differentiation, tolerance, vascular leakage, epithelial mesenchymal transition (EMT), aggregation, angiogenesis, adhesion, development of resistance, addiction to oncogenes and non-oncogenes (cytokines, chemokines, growth factors), alteration of immune surveillance or immune response, alteration of tumor stroma/local environment, endothelial activation, extracellular matrix remodeling, hypoxia and inflammation, immune activation or immune suppression, and survival and/or prevention of cell death by apoptosis, necrosis, or autophagy. In some aspects, the additional agent may be an additional oligomer, which may hybridize to bcl-2 promoter, or to the promoter of another oncogene or disease causing gene.

In other aspects, the chemotherapeutic agent, immunotherapeutic agent, or radiotherapeutic agent is selected from metformin, insulin, 2-deoxyglucose, sulfonylureas, bendamustine, gemcitabine, lenalidomide, aurora A kinase, protease inhibitor, pan-DAC inhibitor, pomalidoide, lenalidomide, cytarabine, fludarabine, CPX-351, cytotoxic agents, anti-diabetic agent, mitochondrial oxidative-phoshorylation uncoupling agent, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), Bruton's tyrosine kinase (BTK) inhibitor, mTOR inhibitors, or agents that influence cancer metabolism, antibodies or compositions that bind or block CD38, CD19, CD30, and CD20, antibodies that stimulate T-cell mediated killing such as PD-1, phosphatidylinositide 3-kinase inhibitors, inhibitors Bruton's tyrosine kinase or spleen tyrosine kinase

In some aspects, the daily dose of oligomer may be from 1 mg/m² to 300 mg/m² oligomer per body surface area of patient. In other aspects, the daily dose of oligomer may be from 1 mg/m² to 200 mg/m² oligomer per body surface area of patient. In some aspects, the daily dose of oligomer and liposome per surface area of the patient together are from about 30 to 150 mg/m². In some aspects, the daily dose of oligomer and liposome per surface area of the patient together are selected from about 30, 40, 50, 60, 70, 80, 90, 100, 110, 120, 130, 140, to 150 mg/m².

In some aspects, the oligonucleotide may be administered via an intravenous infusion to a cancer patient. In some aspects, the oligonucleotide compound is administered intraperitoneally as part of a dialysis regimen. The infusion may be of a duration between 2 hours and 6 hours, or less than two hours.

In some aspects, the administration of the medication occurs before or during administration of the compositions of the present invention. In some aspects, the medication for treatment tolerability may be selected from intravenous, subcutaneous, sublingual, oral or rectally administered electrolyte solutions (e.g., dextrose 5% in water, normal saline), corticosteroids, diphenhydramine, anxiolytics, anti-nausea and anti-diarrheal medications or supportive care measures (e.g., hematologic growth factor support, erythropoiesis-stimulating agents).

In some aspects, the oligomer may be SEQ ID NO:1251.

In some aspects of the present method, the dose may be administered daily for one or more, two or more, three or more, four or more, or five or more days of a dosing cycle, administered daily for 1, 2, 3, 4, 5, 6, 7, 8, 9, 10 or more days of a dosing cycle then weekly thereafter. In some aspects, the dosing cycle may be selected from 15, 18, 19, 20, 21, 22, 23, 24, 25, 28 or 30 days. In some aspects, the dose may be administered on a schedule selected from daily, bidaily, every 2, 3, 4, 5, 6 days, weekly, every 2, 3, 4 weeks, or monthly.

In some aspects of the method, the overall survival rate of patients is improved. In some aspects, the progression-free survival of patients is improved. In some aspects, event-free survival is improved. In some aspects, quality of life is improved. In some aspects, treatment may continue for 1, 2, 3, 4, 5, 6, 7, 8 or more dosing cycles.

Some aspects of the present invention may comprise a method of treating cancer comprising administering to a patient an effective amount of a composition comprising an oligomer of SEQ ID NO:1251 and a liposome comprising POPC/DOPE/MoChol/CHEMS in about a 6/24/47/23 molar ratio, wherein the composition is administered on a dosing cycle selected from 15, 18, 19, 20, 21, 22, 23, 24, 25, 28 or 30 days; wherein the composition is administered daily for 1, 2, 3, 4, 5 or more days of a dosing cycle; and wherein the dose is between about 30 and 150 mg/m² body surface of the subject. In other aspects, the composition is administered on a dosing cycle of 28 days; wherein the composition is administered daily for 2 or more days in the dosing cycle. In some aspects, the dose is 120 mg/m², and wherein the composition is administered IV, on days 1-5 of a 21-day schedule. In some aspects, the present invention administered intravenously, subcutaneously, sublingually, orally or rectally, alone or in combination with chemotherapeutic, immunotherapeutic, radiotherapeutic or surgical interventions. In other aspects, the composition is administered parenterally as a bolus dose or as a continuous infusion for cycles ranging from daily to weekly to monthly as part of an induction or maintenance therapeutic regimen.

Some aspects of the present invention may comprise a method of treating cancer, comprising: administering to a patient an effective amount of an oligonucleotide compound comprising an oligomer that hybridizes under physiological conditions to an oligonucleotide sequence selected from SEQ ID NO:1249 or SEQ ID NO:1254 or the complements thereof, and administering to a patient an effective amount of an additional chemotherapeutic agent, wherein the additional chemotherapeutic agent is selected from alkylating agents (e.g., nitrogen mustards, nitrosoureas, tetrazines, aziridines, cisplatins), anti-metabolites (e.g., anti-folates), anti-microtubule agents (e.g., paclitaxel, vinca alkaloids), topoisomerase inhibitors (e.g., irinotecan, topotecan), cytotoxic antibiotics (e.g., doxorubicin, daunorubicin), metformin, insulin, 2-deoxyglucose, sulfonylureas, anti-diabetic agents generally, mitochondrial oxidative-phoshorylation uncoupling agents, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), mTOR inhibitors, or agents that influence cancer metabolism or cell signal transduction and cell signal pathways, including cell surface, intracellular and secreted proteins, lipids and carbohydrates. In some aspects, the additional chemotherapeutic agent is administered before, simultaneous with, or after the administration of the oligonucleotide compound of claim 1.

### BRIEF DESCRIPTION OF THE DRAWINGS

FIG. 1 depicts the results of a study where PNT2258 and the chemotherapeutic agents rituximab or docetaxel were administered alone or in combination to immunosuppressed mice bearing human tumors.

FIG. 2 depicts the percentage of mice with tumors in partial regression (PR) and/or complete regression (CR), as well as the percentage of animals with tumor-free survival (TFS) at the conclusion of the study depicted in FIG. 1.

FIGS. 3A-D depicts patient data and grouping into initial dosing cohort in a dosing and safety trial in human cancer patient subjects and patient data for a proof of concept single arm study. Patient data is also shown, grouped by cancer type.

FIGS. 4A-C depicts graphs and summary data of PNT2258 concentrations over time in plasma in four representative dose and safety study subjects and area under the curve for all dosing cohorts from 1 mg/m² to 150 mg/m².

FIG. 5 depicts summary data of PNT2258 concentrations over time in plasma of mice study populations.

FIG. 6 depicts the length of time subjects remained in the dose and safety study (measured in days on study), sorted by dosing cohort.

FIGS. 7A-D depicts change in BCL-2 and active BCL-2 expression pre- and post-dose in the dose and safety study subject PBMC cells and change in BCL-2 from pre to post-dose in evaluable single arm proof of concept subject PBMC cells and tumor biopsies.

FIGS. 8A-B depicts the relative amount of BCL-2 knockdown after administration of PNT-2258 in various cancer cell types from the dose and safety study subjects.

FIGS. 9A-C depicts the number of lymphocytes in the human dose and safety study subjects post-administration of various doses of PNT2258 and the human single arm proof of concept subjects post-administration of 120 mg/m² of PNT2258.

FIGS. 10A-B depicts the platelet counts in human dose and safety subjects post-administration of various doses of PNT2258 and the human single arm proof of concept subjects post-administration of 120 mg/m² of PNT2258. The dose-dependent platelet nadir occurs at days 5-9, suggesting effects that are primarily due to megakaryocytes and on-target bcl-2 effect.

FIG. 11 depicts drug interactions between PNT2258, PNT100 and metformin in a Pfeiffer human lymphoma cell line in vitro after 6 days post-administration.

### DETAILED DESCRIPTION

### I. Definitions

As used herein, “patient” refers to a mammal, including a human.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc. and non-vertebrate animals such as Drosophila and C. elegans.

As used herein, an effective amount is defined as the amount required to confer a therapeutic effect on the treated patient, and is typically determined based on age, surface area, weight and condition of the patient. The interrelationship of dosages for animals and humans (based on milligrams per meter squared of body surface) is described by Freireich et al., Cancer Chemother. Rep., 50: 219 (1966). Body surface area can be approximately determined from height and weight of the patient. See, e.g., Scientific Tables, Geigy Pharmaceuticals, Ardsley, N.Y., 537 (1970).

As used herein, the term “wherein said chemotherapy agent is present at less than one half the standard dose” refers to a dosage that is less than one half (e.g., less than 50%, less than 40%, less than 10% or less than 1%) of the minimum value of the standard dosage range used for dosing humans. In some embodiments, the standard dosage range is the dosage range recommended by the manufacturer. In other embodiments, the standard dosage range is the range utilized by a medical doctor in the field. In still other embodiments, the standard dosage range is the range considered the normal standard of care in the field. The particular dosage within the dosage range is determined, for example by the age, weight, and health of the subject as well as the type of cancer being treated.

As used herein, the term “under conditions such that expression of said gene is inhibited” refers to conditions in which an oligonucleotide of the present invention hybridizes to a gene (e.g., a regulatory region of the gene) and inhibits transcription of the gene by at least 10%, at least 25%, at least 50%, or at least 90% relative to the level of transcription in the absence of the oligonucleotide. Exemplary genes include bcl-2; additional genes that may be inhibited along with bcl-2 include, without limitation, c-ki-ras, c-Ha-ras, c-myc, her-2, and TGF-α.

As used herein, the term “under conditions such that growth of said cell is reduced” refers to conditions where an oligonucleotide of the present invention, when administered to a cell (e.g., a cancer) reduces the rate of growth of the cell by at least 10%, at least 25%, at least 50% or at least 90% relative to the rate of growth of the cell in the absence of the oligonucleotide.

As used herein, the term “nucleic acid molecule” refers to any nucleic acid containing molecule, including but not limited to, DNA or RNA. The term encompasses sequences that include any of the known base analogs of DNA and RNA.

The term “gene” refers to a nucleic acid (e.g., DNA) sequence that comprises coding sequences necessary for the production of a polypeptide, precursor or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ or upstream of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the “regulatory region” of a gene is any part of a gene that regulates the expression of a gene, including, without limitation, transcriptional and translational regulation. The regions include without limitation the 5′ and 3′ regions of genes, binding sites for regulatory factors, including without limitation transcription factor binding sites. The regions also include regions that are as long as 20,000 or more base pairs upstream or downstream of translational start sites, so long as the region is involved in any way in the regulation of the expression of the gene. The region may be as short as 20 base pairs or as long as thousands of base pairs.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, micro RNA (miRNA), rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decreases production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 8 and 100), however, as used herein, the term is also intended to encompass longer polynucleotide chains (e.g., as large as 5000 residues). Oligonucleotides are often referred to by their length. For example a 24 residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

In some embodiments, oligonucleotides are “antigens.” As used herein, the term “antigene” refers to an oligonucleotide that hybridizes to the promoter region of a gene. In some embodiments, the hybridization of the antigene to the promoter inhibits expression of the gene.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “completely complementary,” for example when used in reference to an oligonucleotide of the present invention refers to an oligonucleotide where all of the nucleotides are complementary to a target sequence (e.g., a gene).

As used herein, the term “partially complementary,” for example when used in reference to an oligonucleotide of the present invention, refers to an oligonucleotide where at least one nucleotide is not complementary to the target sequence. Exemplary partially complementary oligonucleotides are those that can still hybridize to the target sequence under physiological conditions. The term “partially complementary” refers to oligonucleotides that have regions of one or more non-complementary nucleotides both internal to the oligonucleotide or at either end. Oligonucleotides with mismatches at the ends may still hybridize to the target sequence.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions,” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The present invention is not limited to the hybridization of probes of about 500 nucleotides in length. The present invention contemplates the use of probes between approximately 8 nucleotides up to several thousand (e.g., at least 5000) nucleotides in length. One skilled in the relevant understands that stringency conditions may be altered for probes of other sizes (See e.g., Anderson and Young, Quantitative Filter Hybridization, in Nucleic Acid Hybridization [1985] and Sambrook et al., Molecular Cloning—A Laboratory Manual, Cold Spring Harbor Laboratory, Cold Spring Harbor, N.Y., 2001, and Current Protocols in Molecular Biology, M. Ausubel et al., eds., (Current Protocols, a joint venture between Greene Publishing Associates, Inc. and John Wiley & Sons, Inc., and supplements through 2006.))

It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “physiological conditions” refers to specific stringency conditions that approximate or are conditions inside an animal (e.g., a human). Exemplary physiological conditions for use in vitro include, but are not limited to, 37° C., 95% air, 5% CO₂, commercial medium for culture of mammalian cells (e.g., DMEM media available from Gibco, Md.), 5-10% serum (e.g., calf serum or horse serum), additional buffers, and optionally hormone (e.g., insulin and epidermal growth factor).

As used herein, the term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, recombinant polypeptides are expressed in bacterial host cells and the polypeptides are purified by the removal of host cell proteins; the percent of recombinant polypeptides is thereby increased in the sample.

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants.” An antigenic determinant may compete with the intact antigen (i.e., the “immunogen” used to elicit the immune response) for binding to an antibody.

As used herein, the term “western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “chemotherapeutic agents” refers to compounds that are useful in the treatment of disease (e.g., cancer). Exemplary chemotherapeutic agents affective against cancer include, but are not limited to, daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES), fluradabine, bendamustine, PARP agents, other targeted agents, such as antibodies, or antibody-like agents. Examplary targeted agents may include, for example, inhibitors of kinases, cell surface receptors and proteins/enzymes involved in intracellular and extracellular cell signaling pathways.

Included within the definition of chemotherapeutic agents are compounds useful in augmenting or the effect of a first chemotherapeutic agent or agents or oligonucleotides of the present invention, or mitigating side effects of a first chemotherapeutic agent or agents or oligonucleotide of the present invention.

Included within the definition of immunotherapy are immunomodulating agents that induce, enhance or suppress the immune response.

Included within the definition of radiotherapy are radiological interventions using X-rays, ultrasound, radiowaves, heat or magnetic fields useful in augmenting the effect of a first chemotherapeutic agent or agents or oligonucleotide of the present invention, or mitigating side effects of a first chemotherapeutic agent or agents or oligonucleotide of the present invention.

Included within the definition of surgical therapy are surgical or invasive interventions (e.g., tumor resection, central catheter placement) useful in augmenting the effect of a first chemotherapeutic agent or agents or oligonucleotide of the present invention, or mitigating side effects of a first chemotherapeutic agent or agents or oligonucleotide of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein the term “aliphatic” encompasses the terms alkyl, alkenyl, alkynyl, each of which being optionally substituted as set forth below.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl or 2-ethylhexyl. An alkyl group can be substituted (i.e., optionally substituted) with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, or hydroxy. Without limitation, some examples of substituted alkyls include carboxyalkyl (such as HOOC-alkyl, alkoxycarbonylalkyl and alkylcarbonyloxyalkyl), cyanoalkyl, hydroxyalkyl, alkoxyalkyl, acylalkyl, hydroxyalkyl, aralkyl, (alkoxyaryl)alkyl, (sulfonylamino)alkyl (such as (alkylsulfonylamino)alkyl), aminoalkyl, amidoalkyl, (cycloaliphatic)alkyl, cyanoalkyl, or haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl and 2-hexenyl. An alkenyl group can be optionally substituted with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, (cycloaliphatic)oxy, (heterocycloaliphatic)oxy, aryloxy, heteroaryloxy, aralkyloxy, (heteroaryl)alkoxy, or hydroxy.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as halo, cycloaliphatic, heterocycloaliphatic, aryl, heteroaryl, alkoxy, aroyl, heteroaroyl, (cycloaliphatic)carbonyl, (heterocycloaliphatic)carbonyl, nitro, cyano, amino, amido, acyl, sulfonyl, sulfinyl, sulfanyl, sulfoxy, urea, thiourea, sulfamoyl, sulfamide, oxo, carboxy, carbamoyl, (cycloaliphatic)oxy, (heterocycloaliphatic)oxy, aryloxy, heteroaryloxy, aralkyloxy, (heteroaryl)alkoxy, or hydroxy.

As used herein, an “amido” encompasses both “aminocarbonyl” and “carbonylamino”. These terms when used alone or in connection with another group refers to an amido group such as N(R^{X})₂—C(O)— or R^{Y}C(O)—N(R^{X})₂— when used terminally and —C(O)—N(R^{X})— or —N(R^{X})—C(O)— when used internally, wherein R^{X} and R^{Y} are defined below. Examples of amido groups include alkylamido (such as alkylcarbonylamino and alkylcarbonylamino), (heterocycloaliphatic)amido, (heteroaralkyl)amido, (heteroaryl)amido, (heterocycloalkyl)alkylamido, arylamido, aralkylamido, (cycloalkyl)alkylamido, and cycloalkylamido.

As used herein, an “amino” group refers to —NR^{X}R^{Y} wherein each of R^{X} and R^{Y} is independently hydrogen, alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, and arylamino.

When the term “amino” is not the terminal group (e.g., alkylcarbonylamino), it is represented by —NR^{X}—. R^{X} has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl). The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic) aliphatic)carbonyl; and (heteroaraliphatic)carbonyl]; sulfonyl [e.g., aliphaticsulfonyl and aminosulfonyl]; sulfinyl [e.g., aliphaticsulfinyl]; sulfanyl [e.g., aliphaticsulfanyl]; nitro; cyano; halo; hydroxyl; mercapto; sulfoxy; urea; thiourea; sulfamoyl; sulfamide; and carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((arylalkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl and ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxyl)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; and (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, and bicyclo[3.3.1]nonenyl.

A cycloalkyl or cycloalkenyl group can be optionally substituted with one or more substituents such as aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, and (heteroaraliphatic)carbonylamino], nitro, carboxy [e.g., HOOC—, alkoxycarbonyl, and alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, and (heteroaraliphatic)carbonyl], nitro, cyano, halo, hydroxy, mercapto, sulfonyl [e.g., alkylsulfonyl and arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicyclic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydro-benzofuryl, octahydro-chromenyl, octahydro-thiochromenyl, octahydro-indolyl, octahydro-pyrindinyl, decahydro-quinolinyl, octahydro-benzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline. A “heterocycloalkenyl” group, as used herein, refers to a mono- or bicyclic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, and (heteroaraliphatic)carbonylamino], nitro, carboxy [e.g., HOOC—, alkoxycarbonyl, and alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, and (heteroaraliphatic)carbonyl], nitro, cyano, halo, hydroxy, mercapto, sulfonyl [e.g., alkylsulfonyl and arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring structure having 4 to 15 ring atoms wherein one or more of the ring atoms is a heteroatom (e.g., N, O, S, or combinations thereof) and wherein one ore more rings of the bicyclic or tricyclic ring structure is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thiazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, indolinyl, benzo[b]furyl, benzo[b]thiophenyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); nitro; carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic) aliphatic)carbonyl; and (heteroaraliphatic)carbonyl]; sulfonyl [e.g., aliphaticsulfonyl and amino sulfonyl]; sulfinyl [e.g., aliphaticsulfinyl]; sulfanyl [e.g., aliphaticsulfanyl]; nitro; cyano; halo; hydroxyl; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (amino sulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxyl)heteroaryl; ((carboxy)alkyl)heteroaryl; [((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl, and (haloalkyl)heteroaryl [e.g., trihaloalkylheteroaryl].

A “heteroaraliphatic” (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

As used herein, an “acyl” group refers to a formyl group or R^{X}—C(O)— (such as -alkyl-C(O)—, also referred to as “alkylcarbonyl”) where R^{X} and “alkyl” have been defined previously. Acetyl and pivaloyl are examples of acyl groups.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X} when used as a terminal group or —OC(O)— or —C(O)O—; when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)3- when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —S(O)₂—NR^{X}R^{Y} or —NR^{X}—S(O)₂—R^{Z} when used terminally or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include alkylsulfanyl.

As used herein a “sulfinyl” group refers to —S(O)—R^{X} when used terminally and —S(O)— when used internally, wherein R^{X} has been defined above.

As used herein, a “sulfonyl” group refers to —S(O)₂—R^{X} when used terminally and —S(O)₂— when used internally, wherein R^{X} has been defined above.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) wherein R^{X} and R^{Y} have been defined above.

As used herein, the term “amidino” group refers to the structure —C═(NR^{X})N(R^{X}R^{Y}) wherein R^{X} and R^{Y} have been defined above.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent to at the end of the substituent bound to the to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables contained herein encompass specific groups, such as alkyl and aryl. Unless otherwise noted, each of the specific groups for the variables contained herein can be optionally substituted with one or more substituents described herein. Each substituent of a specific group is further optionally substituted with one to three of halo, cyano, oxoalkoxy, hydroxyl, amino, nitro, aryl, haloalkyl, and alkyl. For instance, an alkyl group can be substituted with alkylsulfanyl and the alkylsulfanyl can be optionally substituted with one to three of halo, cyano, oxoalkoxy, hydroxyl, amino, nitro, aryl, haloalkyl, and alkyl. As an additional example, the cycloalkyl portion of a (cycloalkyl)carbonylamino can be optionally substituted with one to three of halo, cyano, alkoxy, hydroxyl, nitro, haloalkyl, and alkyl. When two alkoxy groups are bound to the same atom or adjacent atoms, the two alkoxy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the foiniation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric focus of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

As used herein, co-therapies include any oligonucleotide compounds that can be used alone or in combination with other cancer therapies to treat cancer.

### II. Cancers

Compounds and methods of the present invention may be used to treat several types of cancer. Examples of cancers that can be treated in some embodiments with compounds and methods of the present invention include solid tumor cancers, including, but not limited to melanoma, metastatic melanoma, non-small cell lung cancer (NSCLC), small cell lung cancer (SCLC), multiple myeloma, chronic lymphocytic leukemia (CLL), small lymphocytic lymphoma (SLL), acute myeloid leukemia (AML), metastatic hormone refractory prostate cancer, breast cancer, ovarian cancer, thyroid cancer, pancreatic cancer, head and neck cancer, and hematological cancers including, but not limited to, all leukemias and lymphomas.

Compounds and methods of the present invention may be used to treat several types of lymphomas subtypes selected from Hodgkin lymphoma, classical Hodgkin lymphoma, lymphocyte-rich/mixed cellularity/lymphocyte depleted, lymphocyte-rich, mixed cellularity, lymphocyte-depleted, nodular sclerosis, classical Hodgkin lymphoma NOS, nodular lymphocyte predominant Hodgkin lymphoma, non-Hodgkin lymphoma, non-Hodgkin lymphoma B-cell, precursor non-Hodgkin lymphoma B-cell, mature non-Hodgkin lymphoma B-cell, chronic/small/prolymphocytic/mantle B-cell NHL, chronic/small lymphocytic leuk/lymph, prolymphocytic leukemia B-cell, mantle-cell lymphoma, lymphoplasmacytic lymphoma/Waldenstrom, lymphoplasmacytic lymphoma, waldenstrom macroglubulinemia, diffuse large B-cell lymphoma (DLBCL), DLBCL NOS, intravascular large B-cell lymphoma, primary effusion lymphoma, mediastinal large B-cell lymphoma, Burkitt lymphoma/leukemia, marginal-zone lymphoma (MZL), splenic MZL, extranodal MZL MALT type, nodal MZL, follicular lymphoma, hairy-cell leukemia, plasma cell neoplasms, plasmacytoma, multiple myeloma/plasma-cell leuk, heavy chain disease, non-Hodgkin lymphoma B-cell NOS, non-Hodgkin lymphoma T-cell, precursor non-Hodgkin lymphoma T-cell, mature Non-Hodgkin lymphoma T-cell, mycosis fungoides/Sezary syndrome, mycosis fungoides, Sezary syndrome, peripheral T-cell lymphoma, peripheral T-cell lymphom NOS, angioimmunoblastic T-cell lymphoma, subcutaneous panniculitis-like T-cell lymphoma, anaplastic large cell lymphoma T- or Null-cell, hepatosplenic T-cell lymphoma, enteropathy-type T-cell lymphoma, cutaneous T-cell lymphoma NOS, primary cutaneous anaplastic large cell lymphoma, adult T-cell leukemia/lymphoma, NK/T-cell lymph., nasal-type/aggressive NK leuk, T-cell large granular lymphocytic leukemia, prolymphocytic leukemia T-cell, non-Hodgkin lymphoma NOS T-cell, non-Hodgkin lymphoma—unknown lineage, precursor lymphoblastic leuk/lymph—unknown lineage, prolymphocytic leukemia—unknown lineage, non-Hodgkin lymphoma NOS—unknown lineage, composite Hodgkin lymphoma and NHL, lymphoid neoplasm NOS, and unclassified subtypes.

Melanoma, or cancer of the skin, is a very common form of cancer, and if diagnosed and treated early can generally be managed. However, if untreated, melanoma can lead to metastatic melanoma and is difficult to treat. Development of stage III or IV melanoma is a serious medical condition and can lead to death usually in 8 to 18 months from the time of diagnosis.

Dacarbazine is the only chemotherapeutic agent approved by the FDA to treat metastatic melanoma, and is associated with a response rate of 7-12% and a median survival of 5.6-7.8 months after the initiation of treatment. Combinations with other chemotherapeutic agents have not shown improvement in response rate. Recently, other agents including ipilimumab, a monoclonal antibody that blocks cytotoxic T-lymphocyte associated antigen 4 (CTLA-4) in combination with dacarbazine, have been shown to have better survival rates than dacarbazine alone. More recently, vemurafenib (PLX4032), a potent inhibitor of mutated BRAF kinase inhibitor showed improved survival in metastatic melanoma patients with the BRAF V600E mutation when compared to dacarbazine.

Approximately 40-60% of cutaneous melanoma carry mutations in the BRAF kinase inhibitor, which leads to the constitutive activation of downstream signaling through the MAPK pathway. Although most (approximately 90%) of the mutations consist of glutamic acid for valine at codon 600 (BRAF V600E), other activating mutations are known, such as BRAF V600K, and BRAF V600R. Targeting the BRAF V600E mutation has lead the discovery and development of vemurafenib and to an improved overall and progression-free survival in patients selected for the BRAF V600E mutation.

However, patients without the BRAF V600E mutation, would appear to have no other treatment alternative other than dacarbazine, the only chemotherapeutic agent approved by the FDA to treat metastatic melanoma. For either treatment choice, the overall survival for any metastatic melanoma patients is generally less than two years.

In other embodiments, the compositions or oligomers of the present invention can be used for treating inflammation disorders such as rheumatoid arthritis, lupis, and inflammatory bowel disease, with or without additional therapeutic agents including TNF-alpha inhibitors such as etanercept, nonsteriodal anti-inflammatory drugs (NSAIDs) such as ibuprofen, corticosteroids, disease modifying antirheumatic drugs (DMARDs) such as methotrexate, and immunosuppressants such as azathioprine, and a CD-20 inhibitor.

### III. Cancer Therapies

Cancer therapies of the present invention include oligonucleotide compounds, chemotherapy agents, radiation therapy, surgery, or combinations thereof.

A. Gene Targets of Oligonucleotide Compounds

1. Bcl-2

In many types of human tumors, including lymphomas and leukemias, the human bcl-2 gene is overexpressed, and may be associated with tumorigenicity (Tsujimoto et al., Science 228:1440-1443 [1985]). Bcl-2 has been found in many forms of both hematologic and solid tumors. These include all solid tumor cancers, including, but not limited to melanoma, metastatic melanoma, non-small cell lung cancer (NSCLC), small cell lung cancer (SCLC), acute myeloid leukemia (AML), metastatic hormone refractory prostate cancer, breast cancer, ovarian cancer, thyroid cancer, pancreatic cancer, head and neck cancer, and hematological cancers including, but not limited to, all leukemias and lymphomas.

High levels of expression of the human bcl-2 gene have been found in all lymphomas with t (14;18) chromosomal translocations including most follicular B cell lymphomas and many large cell non-Hodgkin's lymphomas. High levels of expression of the bcl-2 gene have also been found in certain leukemias that do not have a t(14; 18) chromosomal translation, including most cases of chronic lymphocytic leukemia acute, many lymphocytic leukemias of the pre-B cell type, neuroblastomas, nasopharyngeal carcinomas, and many adenocarcinomas of the prostate, breast and colon. (Reed et al., Cancer Res. 51:6529 [1991]; Yunis et al., New England J. Med. 320:1047; Campos et al., Blood 81:3091-3096 [1993]; McDonnell et al., Cancer Res. 52:6940-6944 [1992]; Lu et al., Int. J Cancer 53:29-35 [1993]; Bonner et al., Lab Invest. 68:43A [1993].).

The current model proposes that BCL-2 proteins work in a hierarchical network of inhibitory interactions to regulate apoptosis. BCL-2 family proteins are essential regulators of apoptosis that contribute to the deregulation of survival pathways in cancer cells. Pro-survival members of the family, such as BCL-2, BCL-XL and MCL-1, possess four BCL-2 homology (BH) domains. Pro-apoptotic BCL-2 proteins are divided into two sub-families Proteins such as BAX or BAK contain BH1-BH3 domains but lack the N-terminal BH4 domain. Proteins such as BAD, BID, BIM or PUMA lack all but the BH3 domain and are known as the ‘BH3-only’ proteins. In healthy cells, the pro-apoptotic effects of BAX and BAK are restrained by the pro-survival proteins BCL-2, BCL-XL and MCL-1.

However, in response to pro-apoptotic stresses, members of the BH3-only proteins are expressed or activated. BH3-only proteins inhibit the pro-survival effects of BCL-2, BCL-XL and MCL-1 thereby liberating the pro-apoptotic effects of BAX and BAK leading to cell death.

The deregulation of apoptosis is a defining characteristic of malignant cells and it is a process in which the overexpression of the BCL-2 protein plays a key role. The elevated BCL2/anti-apoptotic phenotype contributes to the chemo-resistance of a broad variety of tumors including diffuse large B-cell lymphoma and many solid tumors. Given this biological importance, BCL-2 is a prime target for drug discovery. Previous approaches to modulating BCL-2 have included RNA-targeted antisense oligonucleotides, small molecule protein inhibitors and others

2. Other Oncogene Targets

The present invention may include the co-administration of oligonucleotides designed for other oncogene targets, such as c-erb-2 (her-2), c-myc, TGF-α, c-Ha-ras, and c-ki-Ras. Other exemplary oncogenes include, but are not limited to, BCR/ABL, ABL1/BCR, ABL, BCL1, BRAF, CD24, CDK4, EGFR/ERBB-1, HSTF1, INT1/WNT1, INT2, MDM2, MET, MYB, MYC, MYCN, MYCL1, RAF1, NRAS, REL, AKT2, APC, BCL2-ALPHA, BCL2, BCL2-BETA, BCL3, BCR, BRCA1, BRCA2, CBL, CCND1, CDKN1A, CDKN1C, CDKN2A, CDKN2B, CRK, CRK-II, CSF1R/FMS, DBL, DDOST, DCC, DPC4/SMAD4, E-CAD, E2F1/RBAP, ELK1, ELK3, EPH, EPHA1, E2F1, EPHA3, ERG, ETS1, ETS2, FER, FGR, FLI1/ERGB2, FOS, FPS/FES, FRA1, FRA2, FYN, HCK, HEK, HER3/ERBB-2, ERBB-3, HER4/ERBB-4, HST2, INK4A, INK4B, JUN, JUNB, JUND, KIP2, KIT, KRAS2A, KRAS2B, LCK, LYN, MAS, MAX, MCC, MLH1, MOS, MSH2, MYBA, MYBB, NF1, NF2, P53, PDGFB, PIM1, PTC, RB1, RET, ROS1, SKI, SRC1, TAL1, TGFBR2, THRA1, THRB, TIAM1, TRK, VAV, VHL, WAF1, WNT2, WT1, YES1, ALK/NPM1, AMI1, AXL, FMS, GIP, GLI, GSP, HOX11, HST, IL3, INT2, KS3, K-SAM, LBC, LMO-1, LMO-2, L-MYC, LYL1, LYT-10, MDM-2, MLH1, MLL, MLM, N-MYC, OST, PAX-5, PMS-1, PMS-2, PRAD-1, RAF, RHOM-1, RHOM-2, SIS, TAL2, TAN1, TIAM1, TSC2, TRK, TSC1, STK11, PTCH, MEN1, MEN2, P57/KIP2, PTEN, HPC1, ATM, XPA/XPG, BCL6, DEK, AKAP13, CDH1, BLM, EWSR1/FLI1, FES, FGF3, FGF4, FGF6, FANCA, FLI1/ERGB2, FOSL1, FOSL2, GLI, HRAS1, HRX/MLLT1, HRX/MLLT2, KRAS2, MADH4, MAS1, MCF2, MLLT1/MLL, MLLT2/HRX, MTG8/RUNX1, MYCLK1, MYH11/CBFB, NFKB2, NOTCH1, NPM1/ALK, NRG/REL, NTRK1, PBX1/TCF3, PML/RARA, PRCA1, RUNX1, RUNX1/CBFA2T1, SET, TCF3/PBX1, TGFB1, TLX1, P53, WNT1, WNT2, WT1, αv-β3, PKCα, TNFα, Clusterin, Survivin, TGFβ, c-fos, c-SRC, RELA, and INT-1.

3. Non-Oncogene Targets

The present invention is not limited to co-administration of oligonucleotides effective against other oncogenes. For example, in some embodiments, the genes to be targeted include, but are not limited to, an immunoglobulin or antibody gene, a clotting factor gene, a protease, a pituitary hormone, a protease inhibitor, a growth factor, a somatomedian, a gonadotrophin, a chemotactin, a chemokine, a plasma protein, a plasma protease inhibitor, an interleukin, an interferon, a cytokine, a transcription factor, or a pathogen target (e.g., a viral gene, a bacterial gene, a microbial gene, a fungal gene).

Examples of specific genes include, but are not limited to, ADAMTS4, ADAMTS5, APOA1, APOE, APP, B2M, COX2, CRP, DDX25, DMC1, FKBP8, GH1, GHR, IAPP, IFNA1, IFNG, ILL Il10, IL12, IL13, IL2, IL4, IL7, IL8, IPW, MAPK14, Meil, MMP13, MYD88, NDN, PACE4, PRNP, PSEN1, PSEN2, RAD51, RAD51C, SAP, SNRPN, TLR4, TLR9, TTR, UBE3A, VLA-4, and PTP-1B, c-RAF, m-TOR, LDL, VLDL, ApoB-100, VEGF, rhPDGF-BB, NADs, ICAM-1, MUC1, 2-dG, CTL, PSGL-1, E2F, NF-kB, HIF, and GCPRs.

In other embodiments a gene from a pathogen is targeted. Exemplary pathogens include, but are not limited to, Human Immunodeficiency virus, Hepatitis B virus, hepatitis C virus, hepatitis A virus, respiratory syncytial virus, pathogens involved in severe acute respiratory syndrome, West Nile virus and foodborne pathogens (e.g., E. coli).

B. Oligonucleotide Design

In some embodiments, the present invention provides antigene oligonucleotides for inhibiting the expression of oncogenes, such as bcl-2. Exemplary design and production strategies for antigenes are described below. The description below is not intended to limit the scope of antigene compounds suitable for use in the present invention and that other antigenes are within the scope of the present invention.

a. Regulatory Regions of the Oncogenes

The bcl-2 gene has two promoters designated P1 and P2. P1 from which most bcl-2 mRNA is transcribed is located approximately 1.4 kb upstream of the translation initiation site and P2 is 1.3 kb downstream of P1. (See Seto, M. et al. EMBO J. 7, 123-131 (1988).) P1 is GC-rich, lacks a TATA box, has many transcription start sites and includes seven consensus binding sites for the SP1 transcription factor. P2 includes a CCAAT box and a TATA box and has two different transcription initiation sites. There are multiple NF-κB recognition sites and an SV40 enhancer-like octamer motif within P2. (See Heckman, C. A., et al. Oncogene 21, 3898-3908 (2002).) (See SEQ ID NO:1254.) Most human follicular lymphomas contain t(14;18) chromosomal translocations that result from 3′-bcl-2 gene region breakpoints. (See Tsujimoto, Y. et al. Proc. Natl. Acad. Sci. U.S.A 84, 1329-1331 (1987).) These translocations place bcl-2 expression under control of the immunoglobulin heavy chain (IgH) locus enhancer resulting in upregulation of BCL2 expression. Alternatively, there are 5′-bcl-2 breakpoint regions that result from fusions with either the IgH locus or two different immunoglobulin light chain (IgL) loci that are found in some DLCL lymphoma patient isolates. (See Yonetani, N. et al. Jpn. J. Cancer Res. 92, 933-940 (2001).) These 5′-bcl-2 breakpoints have been mapped in separate heterogeneous patient isolates to a region spanning 378 to 2312 bp upstream of the translation initiation site. (See SEQ ID NOs:1255-1266.) The importance of regulatory regions surrounding bcl-2 have been recognized by others. For example, researchers have demonstrated that a series of 20 base deletions between the P1 and P2 promoter of BCL-2 decreased transcription (Young and Korsmeyer Mol. Cell Biol 13: p 3686-3697 (1993) and Chen H M, Boxer L M. Mol Cell Biol. 15: p. 3840-3847 (11995)); Miyashita et. al. reported that p53 dependent regions upstream of the BCL-2 gene act as negative regulatory elements (Cancer Res. 54: p. 3131-3135(1994)); and Duan et. al. showed long range regulatory effects on BCL-2 transcription by enhancers in the IgH 3′ region (Oncogene 27: p. 6720-6728 (2008)). Regions around the breakpoints may be sequences that can be used for bcl-2 oligonucleotide design.

b. Oligonucleotide Design

The oligonucleotides can include any oligomer that hybridizes to the upstream regions of the bcl-2 gene, defined as SEQ ID NOs:1249 and 1254.

In some embodiments, oligonucleotides are designed based on preferred design criteria. Such oligonucleotides can then be tested for efficacy using the methods disclosed herein. For example, in some embodiments, the oligonucleotides are methylated on at least one, two or all of the CpG islands. In other embodiments, the oligonucleotides contain no methylation. The present invention is not limited to a particular mechanism. Indeed, an understanding of the mechanism is not necessary to practice the present invention. Nonetheless, it is contemplated that oligonucleotides in some embodiments are those that have at least a 50% GC content and at least two GC dinucleotides. Also, in some embodiments, the oligonucleotides do not self hybridize. In further embodiments, oligonucleotides are designed with at least 1 A or T to minimize self hybridization. In yet further embodiments, commercially available computer programs are used to survey oligonucleotides for the ability to self hybridize. In still other embodiments, oligonucleotides are at least 10, or 15 nucleotides and no more than 100 nucleotides in length. In further embodiments, oligonucleotides are 18-26 nucleotides in length. In additional embodiments, oligonucleotides comprise the universal protein binding sequences CGCCC and CGCG or the complements thereof.

In some embodiments, oligonucleotides hybridize to a promoter region of a gene upstream from the TATA box of the promoter. In further embodiments, oligonucleotides are designed to hybridize to regions of the promoter region of an oncogene known to be bound by proteins (e.g., transcription factors). In some embodiments, oligonucleotide compounds are not completely homologous to other regions of the human genome. The homology of the oligonucleotide compounds of the present invention to other regions of the genome can be determined using available search tools (e.g., BLAST, available at the Internet site of NCBI).

The present invention is not limited to the oligonucleotides described herein. Other suitable oligonucleotides may be identified (e.g., using the criteria described above or other criteria). Candidate oligonucleotides may be tested for efficacy using any suitable method. For example, candidate oligonucleotides can be evaluated for their ability to prevent cell proliferation at a variety of concentrations. In some embodiments, oligonucleotides inhibit gene expression or cell proliferation at a low concentration (e.g., less than 20 μM, or 10 μM in in vitro assays).

c. Oligonucleotide Zones

In some embodiments, regions within the promoter region of an oncogene are further defined as regions for hybridization of oligonucleotides. In some embodiments, these regions are referred to as “hot zones.”

In some embodiments, hot zones are defined based on oligonucleotide compounds that are demonstrated to be effective (see above section on oligonucleotides) and those that are contemplated to be effective based on the criteria for oligonucleotides described above. In some embodiments, hot zones encompass 10 bp upstream and downstream of each compound included in each hot zone and have at least one CG or more within an increment of 40 bp further upstream or downstream of each compound. In further embodiments, hot zones encompass a maximum of 100 bp upstream and downstream of each oligonucleotide compound included in the hot zone. In additional embodiments, hot zones are defined at beginning regions of each promoter. These hot zones are defined either based on effective sequence(s) or contemplated sequences and have a preferred maximum length of 200 bp. Based on the above described criteria, exemplary hot zones were designed. The hot zones for bcl-2 are located at bases 679-720, 930-1050, 1070-1280, and 1420-1760 of SEQ ID NO:1249.

d. Description

In one aspect, the oligonucleotides can be any oligomer that hybridizes under physiological conditions to the following sequences: SEQ ID NO:1249 or SEQ ID NO:1254. In another aspect, the oligomer can be any oligomer that hybridizes to nucleotides 500-2026, nucleotides 500-1525, nucleotides 800-1225, nucleotides 900-1125, nucleotides 950-1075 or nucleotides 970-1045 of SEQ ID NO:1249 or the complement thereof. In another aspect, the oligonucleotides can be any oligomer that hybridizes under physiological conditions to exemplary hot zones in SEQ ID NO:1249. Examples of oligomers include, without limitation, those oligomers listed in SEQ ID NOS:1250-1253 and 1267-1477 and the complements thereof. In another aspect, the oligonucleotides are SEQ ID NOs 2-22, 283-301, 463-503, 937-958, 1082-1109, 1250-1254 and 1270-1477 and the complements thereof. In an embodiment of these aspects, the oligonucleotides are from 15-35 base pairs in length.

In one embodiment, the oligomer can be SEQ ID NO:1250, 1251, 1252, 1253, 1267-1477 or the complement thereof. In another embodiment, the oligomer can be SEQ ID NO: 1250, 1251, 1267, 1268, 1276, 1277, 1285, 1286 or the complement thereof. In yet another embodiment, the oligomer can be SEQ ID NOs 1250, 1251, 1289-1358 or the complements thereof. In still another embodiment the oligomer can be SEQ ID NO:1250 or 1251.

In a further embodiment of these aspects, the oligomer has the sequence of the positive strand of the bcl-2 sequence, and thus, binds to the negative strand of the sequence.

In other aspects, the oligomers can include mixtures of bcl-2 oligonucleotides. For instance, the oligomer can include multiple oligonucleotides each of which hybridizes to different parts of SEQ ID NOs:1249 and 1254. Oligomers can hybridize to overlapping regions on those sequences or the oligomers may hybridize to non-overlapping regions. In other embodiments, oligomers can be SEQ ID NOs:1250, 1251, 1252, 1253, 1267-1477 or the complement thereof, wherein the mixture of bcl-2 oligomers comprises oligomers of at least 2 different sequences.

In other embodiments, the oligomer can include a mixture of oligomers, each of which hybridizes to a regulatory region of different genes. For instance, the oligomer can include a first oligomer that hybridizes to SEQ ID NO:1249 or 1254 and second oligomer that hybridizes to a regulatory region of a second gene. In some embodiments, the oligomer includes an oligomer of SEQ ID NOs 1250-1254 and 1267-1477 or the complements thereof, In other embodiments, the oligomer includes SEQ ID NO 1250 or 1251 or the complement thereof and an oligomer that hybridizes to the promoter region of another oncogene, such as c-erb-2 (her-2), c-myc, TGF-α, c-Ha-ras, and c-ki-Ras. Examples of such oligomers may be found in, for example, U.S. Pat. Nos. 7,524,827; 7,807,647; and 7,498,315.

In some embodiments, the present invention provides oligonucleotide therapeutics that are methylated at specific sites. The present invention is not limited to a particular mechanism. Indeed, an understanding of the mechanism is not necessary to practice the present invention. Nonetheless, it is contemplated that one mechanism for the regulation of gene activity is methylation of cytosine residues in DNA. 5-methylcytosine (5-MeC) is the only naturally occurring modified base detected in DNA (Ehrlick et al., Science 212:1350-1357 (1981)). Although not all genes are regulated by methylation, hypomethylation at specific sites or in specific regions in a number of genes is correlated with active transcription (Doerfler, Annu. Rev. Biochem. 52:93-124 [1984]; Christman, Curr. Top. Microbiol. Immunol. 108:49-78 [1988]; Cedar, Cell 34:5503-5513 [1988].) DNA methylation in vitro can prevent efficient transcription of genes in a cell-free system or transient expression of transfected genes. Methylation of C residues in some specific cis-regulatory regions can also block or enhance binding of transcriptional factors or repressors (Doerfler, supra; Christman, supra; Cedar, Cell 34:5503-5513 (1988); Tate et al., Curr. Opin. Genet. Dev. 3:225-231 [1993]; Christman et al., Virus Strategies, eds. Doerfler, W. & Bohm, P. (VCH, Weinheim, N.Y.) pp. 319-333 [1993]).

Disruption of normal patterns of DNA methylation has been linked to the development of cancer (Christman et al., Proc. Natl. Acad. Sci. USA 92:7347-7351 [1995]). The 5-MeC content of DNA from tumors and tumor derived cell lines is generally lower than normal tissues (Jones et al., Adv. Cancer Res 40:1-30 [1983]). Hypomethylation of specific oncogenes such as c-myc, c-Ki-ras and c-Ha-ras has been detected in a variety of human and animal tumors (Nambu et al., Jpn. J. Cancer (Gann) 78:696-704 [1987]; Feinberg et al., Biochem. Biophys. Res. Commun. 111:47-54 [1983]; Cheah et al., JNCI73:1057-1063 [1984]; Bhave et al., Carcinogenesis (Lond) 9:343-348 [1988]). In one of the best studied examples of human tumor progression, it has been shown that hypomethylation of DNA is an early event in development of colon cancer (Goetz et al., Science 228:187-290 [1985]). Interference with methylation in vivo can lead to tumor formation. Feeding of methylation inhibitors such as L-methionine or 5-azacytodine or severe deficiency of 5-adenosine methionine through feeding of a diet depleted of lipotropes has been reported to induce formation of liver tumors in rats (Wainfan et al., Cancer Res. 52:2071s-2077s [1992]). Studies show that extreme lipotrope deficient diets can cause loss of methyl groups at specific sites in genes such as c-myc, ras and c-fos (Dizik et al., Carcinogenesis 12:1307-1312 [1991]). Hypomethylation occurs despite the presence of elevated levels of DNA MTase activity (Wainfan et al., Cancer Res. 49:4094-4097 [1989]). Genes required for sustained active proliferation become inactive as methylated during differentiation and tissue specific genes become hypomethylated and are active. Hypomethylation can then shift the balance between the two states. In some embodiments, the present invention thus takes advantage of this naturally occurring phenomena, to provide compositions and methods for site specific methylation of specific gene promoters, thereby preventing transcription and hence translation of certain genes. In other embodiments, the present invention provides methods and compositions for upregulating the expression of a gene of interest (e.g., a tumor suppressor gene) by altering the gene's methylation patterns.

An understanding that mammalian cell promoter regions are surrounded by CpG islands and that these non-methylated regions contribute to gene regulation is emerging (Blackledge N P, Klose R J (2011) Epigenetics 6: p. 147-152 and Deaton A M, Bird A (2011) Genes Dev. 25: p. 1010-1022). These genomic regions surrounding promoters are DNAse I-hypersensitive have also enabled the discovery of cis-regulatory elements that act as transcription factors, enhancers, silencers, repressors, or control regions, which regulate gene expression (Thurman R E, Rynes E, Humbert R, Vierstra H, Maurano M T (2012) Nature 489: 75-82; Maston et al. Annu. Rev. Genomics Hum. Genet. 2006. 7:29-59; Sabo P J, Kuehn M S, Thurman R, Johnson, B E, Johnson, B E et al (2006) Nat Methods 3: p. 511-8). Additionally, higher-order secondary structures (quadruplexes, cruciforms or I-motifs), which surround the promoter regions of oncogenes, may also serve as cis-regulatory domains to modulate transcription (Brazda V, Laister R C, Jagelska E B, Arrowsmith, C (2011) BMC Mol Biol 12: p. 33-48 and Kendrick, S. and L. H. Hurley, Pure Appl Chem, 2010. 82(8): p. 1609-1621. In other embodiments, the present invention provides methods and compositions that can hybridize or bind the hypomethylated or unmethylated CG-rich areas (CpG islands).

The present invention is not limited to the use of methylated oligonucleotides. Indeed, the use of non-methylated oligonucleotides for the inhibition of gene expression is specifically contemplated by the present invention. Experiments conducted during the course of development of the present invention demonstrated that an unmethylated oligonucleotide targeted toward Bcl-2 inhibited the growth of lymphoma cells to a level that was comparable to that of a methylated oligonucleotide.

PNT100, whether unmethylated or methylated, targets an un-transcribed region of the promoter of BCL2 and therefore does not act via translational suppression of BCL2 protein synthesis. Both SEQ ID NOs:1250 and 1251 are included within the scope of the term PNT100 as used below. PNT100 is a 24-base DNA oligonucleotide sequence designed to target a region found within the t(14,18) translocation known to drive certain lymphomas. Subsequent examples use the unmethylated form, but the tem). PNT100 is inclusive of the methylated form.

C. Preparation and Formulation of Oligonucleotides

Any of the known methods of oligonucleotide synthesis can be used to prepare the modified oligonucleotides of the present invention. In some embodiments utilizing methylated oligonucleotides the nucleotide, dC is replaced by 5-methyl-dC where appropriate, as taught by the present invention. The modified or unmodified oligonucleotides of the present invention are most conveniently prepared by using any of the commercially available automated nucleic acid synthesizers. They can also be obtained from commercial sources that synthesize custom oligonucleotides pursuant to customer specifications.

While oligonucleotides are one form of compound, the present invention comprehends other oligomeric oligonucleotide compounds, including but not limited to oligonucleotide mimetics such as are described below. The oligonucleotide compounds in accordance with this invention typically comprise from about 18 to about 30 nucleobases (i.e., from about 18 to about 30 linked bases), although both longer and shorter sequences may find use with the present invention.

Specific examples of compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleotides.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In some embodiments the oligonucleotides have a phosphorothioate backbone having the following general structure.

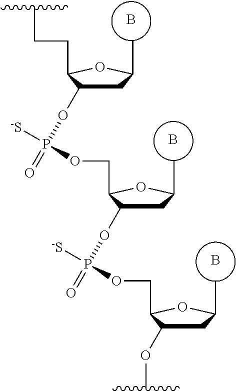

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts.

In other oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991) and Neilsen, Methods in Enzymology, 313, 156-164 (1999). PNA compounds can be obtained commercially, for example, from Applied Biosystems (Foster City, Calif., USA).

In some embodiments, oligonucleotides of the invention are oligonucleotides with phosphorothioate backbones and oligonucleotides with heteroatom backbones, and in particular —CH₂, —NH—O—CH₂—, —CH₂—N(CH₃)—O—CH₂— [known as a methylene(methylimino) or MMI backbone], —CH₂—O—N(CH₃)—CH₂—, —CH₂—N(CH₃)—N(CH₃)—CH₂, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also exemplary are oligonucleotides having morpholino backbone structures of the above-referenced U.S. Pat. No. 5,034,506.

Oligonucleotides can also have sugars other than ribose and deoxyribose, including arabinofuranose (described in International Publication number WO 99/67378, which is herein incorporated by reference), xyloarabinofuranose (described in U.S. Pat. Nos. 6,316,612 and 6,489,465, which are herein incorporated by reference), α-threofuranose (Schöning, et al. (2000) Science, 290, 1347-51, which is herein incorporated by reference) and L-ribofuranose. Sugar mimetics can replace the sugar in the nucleotides. They include cyclohexene (Wang et al. (2000) J. Am. Chem. Soc. 122, 8595-8602; Vebeure et al. Nucl. Acids Res. (2001) 29, 4941-4947, which are herein incorporated by reference), a tricyclo group (Steffens, et al. J. Am. Chem. Soc. (1997) 119, 11548-11549, which is herein incorporated by reference), a cyclobutyl group, a hexitol group (Maurinsh, et al. (1997) J. Org. Chem, 62, 2861-71; J. Am. Chem. Soc. (1998) 120, 5381-94, which are herein incorporated by reference), an altritol group (Allart, et al., Tetrahedron (1999) 6527-46, which is herein incorporated by reference), a pyrrolidine group (Scharer, et al., J. Am. Chem. Soc., 117, 6623-24, which is herein incorporated by reference), carbocyclic groups obtained by replacing the oxygen of the furnaose ring with a methylene group (Froehler and Ricca, J. Am. Chem. Soc. 114, 8230-32, which is herein incorporated by reference) or with an S to obtain 4′-thiofuranose (Hancock, et al., Nucl. Acids Res. 21, 3485-91, which is herein incorporated by reference), and/or morpholino group (Heasman, (2002) Dev. Biol., 243, 209-214, which is herein incorporated by reference) in place of the pentofuranosyl sugar. Morpholino oligonucleotides are commercially available from Gene Tools, LLC (Corvallis Oreg., USA).

The oligonucleotides can also include “locked nucleic acids” or LNAs. The LNAs can be bicyclic, tricyclic or polycyclic. LNAs include a number of different monomers, one of which is depicted in Formula I.

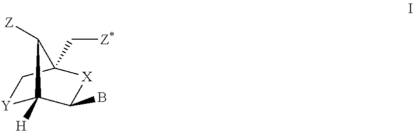

wherein
B constitutes a nucleobase;
Z* is selected from an internucleoside linkage and a terminal group;
Z is selected from a bond to the internucleoside linkage of a preceding nucleotide/nucleoside and a terminal group, provided that only one of Z and Z* can be a terminal group;
X and Y are independently selected from —O—, —S—, —N(H)—, —N(R)—, —CH₂— or —C(H)═, CH₂—O—, —CH₂—S—, —CH₂—N(H)—, —CH₂—N(R)—, —CH₂—CH₂— or —CH₂—C(H)=, —CH═CH—;
   provided that X and Y are not both O. Similarly, oligonucleotides can also include “unlocked nucleic acids” or conformationally unlocked nucleic acids (UNAs).

In addition to the LNA [2′-Y,4′-C-methylene-β-D-ribofuranosyl]monomers depicted in formula I (a [2,2,1]bicyclo nucleoside), an LNA nucleotide can also include “locked nucleic acids” with other furanose or other 5 or 6-membered rings and/or with a different monomer formulation, including 2′-Y,3′ linked and 3′-Y,4′ linked, 1′-Y,3 linked, 1′-Y,4′ linked, 3′-Y,5′ linked, 2′-Y, 5′linked, 1′-Y,2′ linked bicyclonucleosides and others. All the above mentioned LNAs can be obtained with different chiral centers, resulting, for example, in LNA [3′-Y-4′-C-methylene (or ethylene)-β (or α)-arabino-, xylo- or L-ribofuranosyl]monomers. LNA oligonucleotides and LNA nucleotides are generally described in International Publication No. WO 99/14226 and subsequent applications; International Publication Nos. WO 00/56746, WO 00/56748, WO 00/66604, WO 01/25248, WO 02/28875, WO 02/094250, WO 03/006475; U.S. Pat. Nos. 6,043,060, 6,268,490, 6,770,748, 6,639,051, and U.S. Publication Nos. 2002/0125241, 2003/0105309, 2003/0125241, 2002/0147332, 2004/0244840 and 2005/0203042, all of which are incorporated herein by reference. LNA oligonucleotides and LNA analogue oligonucleotides are commercially available from, for example, Proligo LLC, 6200 Lookout Road, Boulder, Colo. 80301 USA.

Oligonucleotides can also contain one or more substituted sugar moieties. Oligonucleotides can comprise one of the following at the 2′ sugar position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Yet other oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide or a group improving pharmacodynamic properties of an oligonucleotide and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further modification includes 2′-dimethylaminooxyethoxy (i.e., an O(CH₂)₂ON(CH₃), group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. A further modification includes constraint ethyl or cET

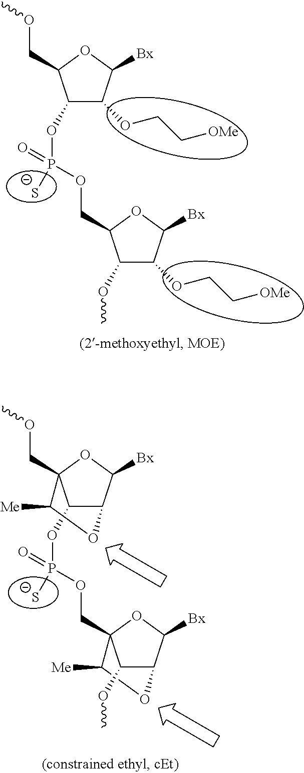

Other modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, isocytosine, pseudoisocytosine, 5-bromouracil, 5-propynyluracil, 5-propynylcytosine, 5-propyny-6-fluoroluracil, 5-methylthiazoleuracil, 6-aminopurine, 2-aminopurine, inosine, diaminopurine, 7-deazaguanine, 7-deazaadenine, 3-deazaguanine, 3-deazaadenine, 8-azaguanine, 8-azaadenine, 7-propyne-7-deazaadenine, 7-propyne-7-deazaguanine, 2-chloro-6-aminopurine, 4-acetylcytosine, 5-hydroxymethylcytosine, 8-hydroxy-N6-methyladenosine, aziridinylcytosine, 5-(carboxyhydroxyl-methyl) uracil, 5-fluorouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, N6-methyladenine, 7-methylguanine and other alkyl derivatives of adenine and guanine, 2-propyl adenine and other alkyl derivatives of adenine and guanine, 2-aminoadenine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 2-thiothymine, 5-halouracil, 5-halocytosine, 6-azo uracil, cytosine and thymine, 5-methyl-2-thiouracil, 2-thiouracil, 4-thiouracil, 5-methyluracil, 8-halo, 8-amino, 8-thiol, 8-hydroxyl and other 8-substituted adenines and guanines, 5-trifluoromethyl uracil and cytosine, N-uracil-5-oxyacetic acid methylester, uracil-5-oxyacetic acid, queosine, xanthine, hypoxanthine, 2-thiocytosine and 2,6-diaminopurine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by −0.6-1.2° C. These are particularly effective when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes pharmaceutical compositions and formulations that include the oligomeric compounds of the present invention as described below.

D. Oligonucleotide Cocktails

In some embodiments, the present invention provides cocktails comprising two or more oligonucleotides directed toward regulatory regions of genes (e.g., oncogenes). In some embodiments, two or more oligonucleotides hybridize to different regions of a regulatory region of the same gene. In other embodiments, the two or more oligonucleotides hybridize to regulatory regions of two different genes. The present invention is not limited to a particular mechanism. Indeed, an understanding of the mechanism is not necessary to practice the present invention. Nonetheless, it is contemplated that the combination of two or more compounds of the present invention provides an inhibition of cancer cell growth that is greater than the additive inhibition of each of the compounds administered separately.

E. Index of SEQ IDs

SEQ ID NO:1249 bcl-2 upstream region

SEQ ID NO:1250 PNT100 oligonucleotide methylated

SEQ ID NO:1251 PNT100 oligonucleotide not methylated

SEQ ID NO:1252 bcl-2 oligonucleotide methylated

SEQ ID NO:1253 bcl-2 oligonucleotide not methylated

SEQ ID NO:1254 bcl-2 secondary promoter sequence

SEQ ID NOs:1255-1266 bcl-2 sequences

SEQ ID NOs:1250-1254 bcl-2 oligonucleotides
and 1267-1477

SEQ ID NOs: 1448-1461 bcl-2 control oligonucleotides

F. Co-Therapies

Oligonucleotide compounds of the present invention can be used alone or in combination with a chemotherapy agent, radiation therapy or surgery.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds include both known and potential therapeutic compounds. A test compound can be determined to be therapeutic by screening using the screening methods of the present invention. In some embodiments of the present invention, test compounds include antisense compounds.

In some embodiments, the oligonucleotide compounds are used or administered with other therapeutic agents such as chemotherapeutic agents, immunotherapeutic agents, or radiotherapeutic agents selected from metformin, insulin, 2-deoxyglucose, sulfonylureas, bendamustine, gemcitabine, lenalidomide, aurora A kinase, protease inhibitor, pan-DAC inhibitor, pomalidoide, lenalidomide, cytarabine, fludarabine, CPX-351, cytotoxic agents, anti-diabetic agent, mitochondrial oxidative-phoshorylation uncoupling agent, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), BTK inhibitor, mTOR inhibitors, or agents that influence cancer metabolism, antibodies or compositions that bind or block CD38, CD19, CD30, and CD20, antibodies that stimulate T-cell mediated killing such as PD-1, phosphatidylinositide 3-kinase inhibitors, inhibitors Bruton's tyrosine kinase (BTK) or spleen tyrosine kinase.

a. Chemotherapy Agents

Chemotherapy agents of the present invention can include any suitable chemotherapy drug or combinations of chemotherapy drugs (e.g., a cocktail). Exemplary chemotherapy agents include, without limitation, alkylating agents, platinums, anti-metabolites, anthracyclines, taxanes, camptothecins, nitrosoureas, EGFR inhibitors, antibiotics, HER2/neu inhibitors, BRAF inhibitors, NRAS or RAS inhibitors, angiogenesis inhibitors, kinase inhibitors, proteaosome inhibitors, immunotherapies, hormone therapies, photodynamic therapies, cancer vaccines, histone deacetylase inhibitors, sphingolipid modulators, oligomers, other unclassified chemotherapy drugs and combinations thereof.

1. Alkylating Agents

Alkylating agents are chemotherapy agents that are thought to attack the negatively charged sites on the DNA (e.g., the oxygen, nitrogen, phosphorous and sulfur atoms) and bind to the DNA thus altering replication, transcription and even base pairing. It is also believed that alkylation of the DNA also leads to DNA strand breaks and DNA strand cross-linking. By altering DNA in this manner, cellular activity is effectively stopped and the cancer cell will die. Common alkylating agents include, without limitation, procarbazine, ifosphamide, cyclophosphamide, bendamustine, melphalan, chlorambucil, dacarbazine, busulfan, thiotepa, and the like. Dacarbazine for Injection is indicated in the treatment of metastatic malignant melanoma. In addition, injections of dacarbazine are also indicated for Hodgkin's disease as a second-line therapy when used in combination with other effective agents. Alkylating agents such as those mentioned above can be used in combination with one or more other alkylating agents and/or with one or more chemotherapy agents of a different class(es).

2. Platinums

Platinum chemotherapy agents are believed to inhibit DNA synthesis, transcription and function by cross-linking DNA subunits. (The cross-linking can happen either between two strands or within one strand of DNA.) Common platinum chemotherapy agents include, without limitation, cisplatin, carboplatin, oxaliplatin, Eloxatin™, and the like. Platinum chemotherapy agents such as those mentioned above can be used in combination with one or more other platinums and/or with one or more chemotherapy agents of a different class(es).

3. Anti-Metabolites

Anti-metabolite chemotherapy agents are believed to interfere with normal metabolic pathways, including those necessary for making new DNA. Common anti-metabolites include, without limitation, Methotrexate, 5-fluorouracil (e.g., capecitabine), gemcitabine (2′-deoxy-2′,2′-difluorocytidine monohydrochloride (β-isomer), Eli Lilly), 6-mercaptopurine, 6-thioguanine, fludarabine, cladribine, cytarabine, tegafur, raltitrexed, cytosine arabinoside, and the like. Gallium nitrate is another anti-metabolite that inhibits ribonucleotides reductase. Anti-metabolites such as those mentioned above can be used in combination with one or more other anti-metabolites and/or with one or more chemotherapy agents of a different class(es).

4. Anthracyclines

Anthracyclines are believed to promote the formation of free oxygen radicals. These radicals result in DNA strand breaks and subsequent inhibition of DNA synthesis and function. Anthracyclines are also thought to inhibit the enzyme topoisomerase by forming a complex with the enzyme and DNA. Common anthracyclines include, without limitation, daunorubicin, doxorubicin, idarubicin, epirubicin, mitoxantrone, adriamycin, bleomycin, mitomycin-C, dactinomycin, mithramycin and the like. Anthracyclines such as those mentioned above can be used in combination with one or more other anthracyclines and/or with one or more chemotherapy agents of a different class(es).

5. Taxanes

Taxanes are believed to bind with high affinity to the microtubules during the M phase of the cell cycle and inhibit their normal function. Common taxanes include, without limitation, paclitaxel, docetaxel (Taxotere™), Taxol™, taxasm, 7-epipaclitaxel, t-acetyl paclitaxel, 10-desacetyl-paclitaxel, 10-desacetyl-7-epipaclitaxel, 7-xylosylpaclitaxel, 10-desacetyl-7-epipaclitaxel, 7-N—N-dimethylglycylpaclitaxel, 7-L-alanylpaclitaxel and the like. Taxanes such as those mentioned above can be used in combination with one or more other taxanes and/or with one or more chemotherapy agents of a different class(es).

For instance, Taxotere™ is indicated for the treatment of patients with locally advanced or metastatic breast cancer after failure of prior chemotherapy; in combination with doxorubicin and cyclophosphamide is indicated for the adjuvant treatment of patients with operable node-positive breast cancer; as a single agent, is indicated for the treatment of patients with locally advanced or metastatic non-small cell lung cancer (NSCLC) after failure of prior platinum-based chemotherapy; in combination with cisplatin is indicated for the treatment of patients with unresectable, locally advanced or metastatic NSCLC who have not previously received chemotherapy for this condition; in combination with prednisone is indicated for the treatment of patients with androgen-independent (hormone-refractory) metastatic prostate cancer; in combination with cisplatin and fluorouracil is indicated for the treatment of patients with advanced gastric adenocarcinoma, including adenocarcinoma of the gastroesophageal junction, who have not received prior chemotherapy for advanced disease; and in combination with cisplatin and fluorouracil is indicated for the induction treatment of patients with locally advanced squamous cell carcinoma of the head and neck (SCCHN).

6. Camptothecins

Camptothecins are thought to complex with topoisomerase and DNA resulting in the inhibition and function of this enzyme. It is further believed that the presence of topoisomerase is required for on-going DNA synthesis. Common camptothecins include, without limitation, irinotecan, topotecan, etoposide, vinca alkaloids (e.g., vincristine, vinblastine or vinorelbine), amsacrine, teniposide and the like. Camptothecins such as those mentioned above can be used in combination with one or more other camptothecins and/or with one or more chemotherapy agents of a different class(es).

7. Nitrosoureas

Nitrosoureas are believed to inhibit changes necessary for DNA repair. Common nitrosoureas include, without limitation, carmust

ine (BCNU), lomustine (CCNU), semustine and the like. Nitrosoureas such as those mentioned above can be used in combination with one or more other nitrosoureas and/or with one or more chemotherapy agents of a different class(es).

8. EGFR Inhibitors

EGFR (i.e., epidermal growth factor receptor) inhibitors are thought to inhibit EGFR and interfere with cellular responses including cell proliferation and differentiation. EGFR inhibitors include molecules that inhibit the function or production of one or more EGFRs. They include small molecule inhibitors of EGFRs, antibodies to EGFRs, antisense oligomers, RNAi inhibitors and other oligomers that reduce the expression of EGFRs. Common EGFR inhibitors include, without limitation, gefitinib, erlotinib (Tarceva®), cetuximab (Erbitux™), panitumumab (Vectibix®, Amgen) lapatinib (GlaxoSmithKline), CI1033 or PD183805 or canternib (6-acrylamide-N-(3-chloro-4-flurorphenyl)-7-(3-morpholinopropoxy)quinazolin-4-amine, Pfizer), and the like. Other inhibitors include PKI-166 (4-[(1R)-1-phenylethylamino]-6-(4-hydroxyphenyl)-7H-pyrrolo[2,3-d]pyrimidine, Novartis), CL-387785 (N-[4-(3-bromoanilino)quinazolin-6-yl]but-2-ynamide), EKB-569 (4-(3-chloro-4-fluroranilino)-3-cyano-6-(4-dimethylaminobut2(E)-enamido)-7-ethoxyquinoline, Wyeth), lapatinib (GW2016, GlaxoSmithKline), EKB509 (Wyeth), panitumumab (ABX-EGF, Abgenix), matuzumab (EMD 72000, Merck), and the monoclonal antibody RH3 (New York Medical). EGFR inhibitors such as those mentioned above can be used in combination with one or more other EGFR inhibitors and/or with one or more chemotherapy agents of a different class(es).

9. Antibiotics

Antibiotics are thought to promote the formation of free oxygen radicals that result in DNA breaks leading to cancer cell death. Common antibiotics include, without limitation, bleomycin and rapamycin and the like. The macrolide fungicide rapamycin (also called RAP, rapamune and sirolimus) binds intracellularly to the to the immunophilin FK506 binding protein 12 (FKBP12) and the resultant complex inhibits the serine protein kinase activity of mammalian target of rapamycin (mTOR). Rapamycin macrolides include naturally occurring forms of rapamycin as well as rapamycin analogs and derivatives that target and inhibit mTOR. Other rapamycin macrolides include, without limitation, temsirolimus (CCI-779, Wyeth), everolimus and ABT-578. Antibiotics such as those mentioned above can be used in combination with one or more other antibiotics and/or with one or more chemotherapy agents of a different class(es).

10. HER2/neu Inhibitors

HER2/neu Inhibitors are believed to block the HER2 receptor and prevent the cascade of reactions necessary for tumor survival. Her2 inhibitors include molecules that inhibit the function or production of Her2. They include small molecule inhibitors of Her2, antibodies to Her2, antisense oligomers, RNAi inhibitors and other oligomers that reduce the expression of tyrosine kinases. Common HER2/neu inhibitors include, without limitation, trastuzumab (Herceptin®, Genentech) and the like. Other Her2/neu inhibitors include bispecific antibodies MDX-210(FCγR1-Her2/neu) and MDX-447 (Medarex), pertuzumab (rhuMAb 2C4, Genentech), HER2/neu inhibitors such as those mentioned above can be used in combination with one or more other HER2/neu inhibitors and/or with one or more chemotherapy agents of a different class(es).

11. Angiogenesis Inhibitors

Angiogenesis inhibitors are believed to inhibit vascular endothelial growth factor, i.e., VEGF, thereby inhibiting the formation of new blood vessels necessary for tumor life. VEGF inhibitors include molecules that inhibit the function or production of one or more VEGFs. They include small molecule inhibitors of VEGF, antibodies to VEGF, antisense oligomers, RNAi inhibitors and other oligomers that reduce the expression of tyrosine kinases. Common angiogenesis inhibitors include, without limitation, bevacizumab (Avastin®, Genentech). Other angiogenesis inhibitors include, without limitation, ZD6474 (AstraZeneca), BAY-43-9006, sorafenib (Nexavar®, Bayer), semaxanib (SU5416, Pharmacia), SU6668 (Pharmacia), ZD4190 (N-(4-bromo-2-fluorophenyl)-6-methoxy-7-[2-(1H-1,2,3-triazol-1-yl)ethoxy]quinazolin-4-amine, Astra Zeneca), Zactima™ (ZD6474, N-(4-bromo-2-fluorophenyl)-6-methoxy-7-[2-(1H-1,2,3-triazol-1-yl)ethoxy]quinazolin-4-amine, Astra Zeneca), vatalanib, (PTK787, Novartis), the monoclonal antibody IMC-1C11 (Imclone) and the like. Angiogenesis inhibitors such as those mentioned above can be used in combination with one or more other angiogenesis inhibitors and/or with one or more chemotherapy agents of a different class(es).

12. BRAF Inhibitors

The B-Raf (BRAF) variant, BRAF V600E, is the most frequent oncogenic protein kinase mutation known. The selection of potent and selective inhibitory agents to active BRAF V600E has led to a number of agents that show BRAF kinase specificity and cytotoxic effects to cells bearing the BRAF V600E mutation. In particular, the Plexxikon agent, PLX4720, was reported as demonstrating specific ERK phosphorylation in BRAF V600E but not BRAF wild-type tumor cells. In melanoma models, PLX4720 induced cell cycle arrest and apoptosis in B-Raf V600E positive cells. The Plexxikon agent, vemurafenib (PLX4032), another B-Raf V600E specific agent, was tested in humans with metastatic melanoma with the BRAF V600E. A significant treatment effect was observed for improved overall survival and progression free survival.

As noted above, although most (approximately 90%) of the mutations consist of glutamic acid for valine at codon 600 (BRAF V600E), other activating mutations are known, such as BRAF V600K, and BRAF V600R.

BRAF V600E and “wild-type” BRAF has been associated many cancers, including for example, Non-Hodgkin's lymphoma, leukemia, malignant melanoma, thyroid, colorectal, and adenocarcinoma and NSCLC.

Other BRAF inhibitors that may be used in embodiments of the present invention include, but are not limited to, GDC-0879, BAY 7304506 (regorafenib), RAF265 (CHIR-265), SB590885, Sorafenib.

13. Other Kinase Inhibitors

In addition to EGFR, HER2, BRAF and VEGF inhibitors, other kinase inhibitors are used as chemotherapeutic agents. Aurora kinase inhibitors include, without limitation, compounds such as 4-(4-N benzoylamino)aniline)-6-methyxy-7-(3-(1-morpholino)propoxy)quinazoline (ZM447439, Ditchfield et al., J. Cell. Biol., 161:267-80 (2003)) and hesperadin (Haaf et al., J. Cell Biol., 161: 281-94 (2003)). Other compounds suitable for use as Aurora kinase inhibitors are described in Vankayalapati H, et al., Mol. Cancer Ther. 2:283-9 (2003). SRC/Abl kinase inhibitors include without limitation, AZD0530 (4-(6-chloro-2,3-methylenedioxyanilino)-7-[2-(4-methylpiperazin-1-yl)ethoxy]-5-tetrahycropyran-4-yloxyquinazoline). Tyrosine kinase inhibitors include molecules that inhibit the function or production of one or more tyrosine kinases. They include small molecule inhibitors of tyrosine kinases, antibodies to tyrosine kinases and antisense oligomers, RNAi inhibitors and other oligomers that reduce the expression of tyrosine kinases. CEP-701 and CEP-751 (Cephalon) act as tyrosine kinase inhibitors. Imatinib mesylate is a tyrosine kinase inhibitor that inhibits bcr-abl by binding to the ATP binding site of bcr-abl and competitively inhibiting the enzyme activity of the protein. Although imatinib is quite selective for bcr-abl, it does also inhibit other targets such as c-kit and PDGF-R. FLT-3 inhibitors include, without limitation, tandutinib (MLN518, Millenium), sutent (SU11248, 5-[5-fluoro-2-oxo-1,2-dihydroindol-(3Z)-ylidenemethyl]-2,4-dimethyl-1H-pyrrole-3-carboxylic acid [2-diethylaminoethyl]amide, Pfizer), midostaurin (4′-N-benzoyl staurosporine, Novartis), lefunomide (SU101) and the like. MEK inhibitors include, without limitation, 2-(2-Chloro-4-iodo-phenylamino)-N-cyclopropylmethoxy-3,4-difluoro-benzamide (PD184352/CI-1044, Pfizer), PD198306 (Pfizer), PD98059 (2′-amino-3′-methoxyflavone), UO126 (Promega), Ro092-210 from fermented microbial extracts (Roche), the resorcyclic acid lactone, L783277, also isolated from microbial extracts (Merck) and the like. Tyrosine kinase inhibitors such as those mentioned above can be used in combination with one or more other tyrosine kinase inhibitors and/or with one or more chemotherapy agents of a different class(es) including phosphatidylinositide 3-kinase inhibitors, Bruton's tyrosine kinase inhibitors and spleen tyrosine kinase (also known as Syk protein (encoded by the SYK gene)) inhibitors without limitation.

14. Proteaosome Inhibitors

Proteaosome inhibitors are believed to inhibit the breakdown of some of these proteins that have been marked for destruction. This results in growth arrest or death of the cell. Common proteaosome inhibitors include, without limitation, bortezomib, ortezomib and the like. Proteaosome inhibitors such as those mentioned above can be used in combination with one or more other proteaosome inhibitors and/or with one or more chemotherapy agents of a different class(es).

15. Immunotherapies

Immunotherapies are thought to bind to and block specific targets, thereby disrupting the chain of events needed for tumor cell proliferation. Common immunotherapies include, without limitation, rituximab and other antibodies directed against CD20, Campath-1H™ and other antibodies directed against CD-50, epratuzmab and other antibodies directed against CD-22, galiximab and other antibodies directed against CD-80, apolizumab HU1D10 and other antibodies directed against HLA-DR, and the like. Radioisotopes can be conjugated to the antibody, resulting in radioimmunotherapy. Two such anti-CD20 products are tositumomab (Bexxar™) and ibritumomab (Zevalin™) Immunotherapies such as those mentioned above can be used in combination with one or more other immunotherapies and/or with one or more chemotherapy agents of a different class(es). Antibodies or compositions that bind or block CD38, CD19 and CD20 and antibodies that stimulate T-cell mediated killing such as PD-1.

Rituximab (Rituxan™), among other indications, is indicated for the treatment of patients with previously untreated follicular, CD20-positive, B-cell non-Hodgkin's lymphoma; and previously untreated and previously treated CD20-positive chronic lymphocytic leukemia in combination with fludarabine and cyclophosphamide (FC).

Yervoy™ (ipilimumab) is a monoclonal antibody that blocks a molecule known as cytotoxic T-lymphocyte antigen or CTLA-4. CTLA-4 may play a role in slowing down or turning off the body's immune system, affecting its ability to fight off cancerous cells. Yervoy may work by allowing the body's immune system to recognize, target, and attack cells in melanoma tumors. The drug is administered intravenously. Yervoy is indicated for the treatment of unresectable or metastatic melanoma. Yervoy (3 mg/kg) is administered intravenously over 90 minutes every 3 weeks for a total of four doses. Two key clinical trials have been conducted with Yervoy. The first which resulted in FDA approval based on Yervoy's safety and effectiveness in a single international study of 676 patients with melanoma. All patients in the study had stopped responding to other FDA-approved or commonly used treatments for melanoma. In addition, participants had disease that had spread or that could not be surgically removed.

Other CTLA-4 antibodies, which may be used in embodiments of the present invention include, but are not limited to tremelimumab.

16. Hormone Therapies

Hormone therapies are thought to block cellular receptors, inhibit the in vivo production of hormones, and/or eliminate or modify hormone receptors on cells, all with the end result of slowing or stopping tumor proliferation. Common hormone therapies include, without limitation, antiestrogens (e.g., tamoxifen, toremifene, fulvestrant, raloxifene, droloxifene, idoxifene and the like), progestogens e.g., megestrol acetate and the like) aromatase inhibitors (e.g., anastrozole, letrozole, exemestane, vorozole, exemestane, fadrozole, aminoglutethimide, exemestane, 1-methyl-1,4-androstadiene-3,17-dione and the like), anti-androgens (e.g., bicalutimide, nilutamide, flutamide, cyproterone acetate, and the like), luteinizing hormone releasing hormone agonist (LHRH Agonist) (e.g., goserelin, leuprolide, buserelin and the like); 5-α-reductase inhibitors such as finasteride, and the like.

Abiraterone (Zytiga™) is another useful hormone therapy, which inhibits the enzyme 17 α-hydroxylase/C17,20 lyase in testicular, prostate, and adrenal cancer tissue, blocking the synthesis of precursors of testosterone. Hormone therapies such as those mentioned above can be used in combination with one or more other hormone therapies and/or with one or more chemotherapy agents of a different class(es).

17. Photodynamic Therapies

Photodynamic therapies expose a photosensitizing drug to specific wavelengths of light to kill cancer cells. Common photodynamic therapies include, for example, porfimer sodium (e.g., Photofrin®) and the like. Photodynamic therapies such as those mentioned above can be used in combination with one or more other photodynamic therapies and/or with one or more chemotherapy agents of a different class(es).

18. Cancer Vaccines

Cancer vaccines are thought to utilize whole, inactivated tumor cells, whole proteins, peptide fragments, viral vectors and the like to generate an immune response that targets cancer cells. Common cancer vaccines include, without limitation, modified tumor cells, peptide vaccine, dendritic vaccines, viral vector vaccines, heat shock protein vaccines and the like.

19. Histone Deacetylase Inhibitors

Histone deacetylase inhibitors are able to modulate transcriptional activity and consequently, can block angiogenesis and cell cycling, and promote apoptosis and differentiation. Histone deacetylase inhibitors include, without limitation, SAHA (suberoylanilide hydroxamic acid), depsipeptide (FK288) and analogs, Pivanex™ (Titan), CI994 (Pfizer), MS275 PXD101 (CuraGen, TopoTarget) MGCD0103 (MethylGene), LBH589, NVP-LAQ824 (Novartis) and the like and have been used as chemotherapy agents. Histone deacetylase inhibitors such as those mentioned above can be used in combination with one or more other histone deacetylase inhibitors and/or with one or more chemotherapy agents of a different class(es).

20. Sphingolipid Modulators

Modulators of Sphingolipid metabolism have been shown to induce apoptosis. For reviews see N. S. Radin, Biochem J, 371:243-56 (2003); D. E. Modrak, et al., Mol. Cancer Ther, 5:200-208 (2006), K. Desai, et al., Biochim Biophys Acta, 1585:188-92 (2002) and C. P. Reynolds, et al. and Cancer Lett, 206, 169-80 (2004), all of which are incorporated herein by reference. Modulators and inhibitors of various enzymes involved in sphingolipid metabolism can be used as chemotherapeutic agents.

(a) Ceramide has been shown to induce apoptosis, consequently, exogenous ceramide or a short-chain ceramide analog such as N-acetylsphingosine (C₂-Cer), C₆-Cer or C₃-Cer has been used. Other analogs include, without limitation, Cer 1-glucuronide, poly(ethylene glycol)-derivatized ceramides and pegylated ceramides.

(b) Modulators that stimulate ceramide synthesis have been used to increase ceramide levels. Compounds that stimulate serine palmitoyltransferase, an enzyme involved in ceramide synthesis, include, without limitation, tetrahydrocannabinol (THC) and synthetic analogs and anandamide, a naturally occurring mammalian cannabinoid. Gemcitabine, retinoic acid and a derivative, fenretinide[N-(4-hydroxyphenyl)retinamide, (4-HPR)], camptothecin, homocamptothecin, etoposide, paclitaxel, daunorubicin and fludarabine have also been shown to increase ceramide levels. In addition, valspodar (PSC833, Novartis), a non-immunosuppressive non-ephrotoxic analog of cyclosporin and an inhibitor of p-glycoprotein, increases ceramide levels.

(c) Modulators of sphingomyelinases can increase ceramide levels. They include compounds that lower GSH levels, as GSH inhibits sphingomyelinases. For example, betathine (β-alanyl cysteamine disulfide), oxidizes GSH, and has produced good effects in patients with myeloma, melanoma and breast cancer. COX-2 inhibitors, such as celecoxib, ketoconazole, an antifungal agent, doxorubicin, mitoxantrone, D609 (tricyclodecan-9-yl-xanthogenate), dexamethasone, and Ara-C(1-β-D-arabinofuranosylcytosine) also stimulate sphingomyelinases.

(d) Molecules that stimulate the hydrolysis of glucosylceramide also raise ceramide levels. The enzyme, GlcCer glucosidase, which is available for use in Gaucher's disease, particularly with retinol or pentanol as glucose acceptors and/or an activator of the enzyme can be used as therapeutic agents. Saposin C and analogs thereof, as well as analogs of the anti-psychotic drug, chloropromazine, may also be useful.

(e) Inhibitors of glucosylceramide synthesis include, without limitation, PDMP (N-[2-hydroxy-1-(4-morpholinylmethyl)-2-phenylethyldecanamide]), PMPP (D,L-threo-(1-phenyl-2-hexadecanoylamino-3-morpholino-1-propanol), P4 or PPPP (D-threo-1-phenyl-2-palmitoylamino-3-pyrrolidino-1-propanol), ethylenedioxy-P4, 2-decanoylamine-3-morpholinoprophenone, tamixofen, raloxifene, mifepristone (RU486), N-butyl deoxynojirimycin and anti-androgen chemotherapy (bicalutamide+leuprolide acetate)). Zavesca®, (1,5-(butylimino)-1,5-dideoxy-D-glucitol) usually used to treat Gaucher's disease, is another inhibitor of glucosylceramide synthesis.

(f) Inhibitors of ceramidase include, without limitation, N-oleoylethanolamine, a truncated form of ceramide, D-MAPP (D-erythro-2-tetradecanoylamino-1-phenyl-1-propanol) and the related inhibitor B13 (p-nitro-D-MAPP).

(g) Inhibitors of sphingosine kinase also result in increased levels of ceramide. Inhibitors include, without limitation, safingol (L-threo-dihydrosphingosine), N,N-dimethyl sphingosine, trimethyl sphingosine and analogs and derivatives of sphingosine such as dihydrosphingosine, and myriocin.

(h) Fumonisins and fumonisin analogs, although they inhibit ceramide synthase, also increase levels of sphinganine due to the inhibition of de novo sphingolipid biosynthesis, resulting in apoptosis.

(i) Other molecules that increase ceramide levels include, without limitation, miltefosine (hexadecylphosphocholine). Sphingolipid modulators, such as those mentioned above, can be used in combination with one or more other sphingolipid modulators and/or with one or more chemotherapy agents of a different class(es).

21. Other Oligomers

In addition to the oligonucleotides presented above, other oligonucleotides have been used as cancer therapies. They include Genasense® (oblimersen, G3139, from Genta), an antisense oligonucleotide that targets bcl-2 and G4460 (LR3001, from Genta) another antisense oligonucleotides that targets cancer pathways including, but not limited to STAT-3, survivin, c-myb and others. Other oligomers include, without limitation, siRNAs, decoys, RNAi oligonucleotides and the like. Oligonucleotides, such as those mentioned above, can be used in combination with one or more other oligonucleotide inhibitors and/or with one or more chemotherapy agents of a different class(es).

22. Other Chemotherapy Drugs

Additional unclassified chemotherapy agents are described in Table 1 below.

TABLE 1
Additional unclassified chemotherapy agents.

| Generic Name | Brand Name | Manufacturer |
|---|---|---|
| aldesleukin | Proleukin ™ | Chiron Corp., |
| (des-alanyl-1, serine-125 human interleukin-2) | | Emeryville, CA |
| alemtuzumab | Campath ™ | Millennium and |
| (IgG1κ anti CD52 antibody) | | ILEX Partners, LP, |
| | | Cambridge, MA |
| alitretinoin | Panretin ™ | Ligand |
| (9-cis-retinoic acid) | | Pharmaceuticals, |
| | | Inc., San Diego CA |
| allopurinol | Zyloprim ™ | GlaxoSmithKline, |
| (1,5-dihydro-4H-pyrazolo[3,4-d]pyrimidin-4- | | Research Triangle |
| one monosodium salt) | | Park, NC |
| altretamine | Hexalen ™ | US Bioscience, |
| (N,N,N′,N′,N″,N″,- hexamethyl-1,3,5-triazine- | | West |
| 2, 4, 6-triamine) | | Conshohocken, PA |
| amifostine | Ethyol ™ | US Bioscience |
| (ethanethiol, 2-[(3-aminopropyl)amino]-, | | |
| dihydrogen phosphate (ester)) | | |
| anastrozole | Arimidex ™ | AstraZeneca |
| (1,3-Benzenediacetonitrile, a, a, a′, a′- | | Pharmaceuticals, |
| tetramethyl-5-(1H-1,2,4-triazol-1-ylmethyl)) | | LP, Wilmington, |
| | | DE |
| arsenic trioxide | Trisenox ™ | Cell Therapeutic, |
| | | Inc., Seattle, WA |
| asparaginase | Elspar ™ | Merck & Co Inc., |
| (L-asparagine amidohydrolase, type EC-2) | | Whitehouse Station, |
| | | NJ |
| BCG Live | TICE BCG ™ | Organon Teknika, |
| (lyophilized preparation of an attenuated strain | | Corp., Durham, NC |
| of Mycobacterium bovis (Bacillus Calmette- | | |
| Gukin [BCG], substrain Montreal) | | |
| bexarotene capsules | Targretin ™ | Ligand |
| (4-[1-(5,6,7,8-tetrahydro-3,5,5,8,8- | | Pharmaceuticals |
| pentamethyl-2-napthalenyl) ethenyl] benzoic acid) | | |
| bexarotene gel | Targretin ™ | Ligand |
| | | Pharmaceuticals |
| carmustine with polifeprosan 20 implant | Gliadel Wafer ™ | Guilford |
| | | Pharmaceuticals, |
| | | Inc., Baltimore, MD |
| celecoxib | Celebrex ™ | Searle |
| (as 4-[5-(4-methylphenyl)-3-(trifluoromethyl)- | | Pharmaceuticals, |
| 1H-pyrazol-1-yl] benzenesulfonamide) | | England |
| chlorambucil | Leukeran ™ | GlaxoSmithKline |
| (4-[bis(2chlorethyl)amino]benzenebutanoic acid) | | |
| cladribine | Leustatin, 2- | R.W. Johnson |
| (2-chloro-2′-deoxy-b-D-adenosine) | CdA ™ | Pharmaceutical |
| | | Research Institute, |
| | | Raritan, NJ |
| dacarbazine | DTIC-Dome ™ | Bayer AG, |
| (5-(3,3-dimethyl-1-triazeno)-imidazole-4- | | Leverkusen, |
| carboxamide (DTIC)) | | Germany |
| dactinomycin, actinomycin D | Cosmegen ™ | Merck |
| (actinomycin produced by Streptomyces | | |
| parvullus, C₆₂H₃₆N₁₂O₁₆) | | |
| darbepoetin alfa | Aranesp ™ | Amgen, Inc., |
| (recombinant peptide) | | Thousand Oaks, CA |
| denileukin diftitox | Ontak ™ | Seragen, Inc., |
| (recombinant peptide) | | Hopkinton, MA |
| dexrazoxane | Zinecard ™ | Pharmacia & |
| ((S)-4,4′-(1-methyl-1,2-ethanediyl)bis-2,6- | | Upjohn Company |
| piperazinedione) | | |
| dromostanolone propionate | Dromostanolone ™ | Eli Lilly & |
| (17b-Hydroxy-2a-methyl-5a-androstan-3-one | | Company, |
| propionate) | | Indianapolis, IN |
| dromostanolone propionate | Masterone | Syntex, Corp., Palo |
| | injection ™ | Alto, CA |
| Elliott's B Solution | Elliott's B | Orphan Medical, |
| | Solution ™ | Inc |
| epoetin alfa | Epogen ™ | Amgen, Inc |
| (recombinant peptide) | | |
| estramustine | Emcyt ™ | Pharmacia & |
| (estra-1,3,5(10)-triene-3,17-diol(17(beta))-, 3- | | Upjohn Company |
| [bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| exemestane | Aromasin ™ | Pharmacia & |
| (6-methylenandrosta-1,4-diene-3, 17-dione) | | Upjohn Company |
| filgrastim | Neupogen ™ | Amgen, Inc |
| (r-metHuG-CSF) | | |
| floxuridine (intraarterial) | FUDR ™ | Roche |
| (2′-deoxy-5-fluorouridine) | | |
| fulvestrant | Faslodex ™ | IPR |
| (7-alpha-[9-(4,4,5,5,5-penta | | Pharmaceuticals, |
| fluoropentylsulphinyl) nonyl]estra-1,3,5-(10)- | | Guayama, Puerto |
| triene-3,17-beta-diol) | | Rico |
| gemtuzumab ozogamicin | Mylotarg ™ | Wyeth Ayerst |
| (anti-CD33 hP67.6) | | |
| hydroxyurea | Hydrea ™ | Bristol-Myers |
| | | Squibb |
| ifosfamide | IFEX ™ | Bristol-Myers |
| (3-(2-chloroethyl)-2-[(2- | | Squibb |
| chloroethyl)amino]tetrahydro-2H-1,3,2- | | |
| oxazaphosphorine 2-oxide) | | |
| imatinib mesilate | Gleevec ™ | Novartis AG, Basel, |
| (4-[(4-Methyl-1-piperazinyl)methyl]-N-[4- | | Switzerland |
| methyl-3-][4-(3-pyridinyl)-2- | | |
| pyrimidinyl]amino]-phenyl]benzamide | | |
| methanesulfonate) | | |
| interferon alpha-2a | Roferon-A ™ | Hoffmann-La |
| (recombinant peptide) | | Roche, Inc., Nutley, |
| | | NJ |
| interferon alpha-2b | Intron A ™ | Schering AG, |
| (recombinant peptide) | (Lyophilized | Berlin, Germany |
| | Betaseron) | |
| irinotecan HCl | Camptosar ™ | Pharmacia & |
| ((4S)-4,11-diethyl-4-hydroxy-9-[(4-piperi- | | Upjohn Company |
| dinopiperidino)carbonyloxy]-1H-pyrano[3′, 4′: | | |
| 6,7] indolizino[1,2-b] quinoline-3,14(4H, 12H) | | |
| dione hydrochloride trihydrate) | | |
| letrozole | Femara ™ | Novartis |
| (4,4′-(1H-1,2,4-Triazol-1-ylmethylene) | | |
| dibenzonitrile) | | |
| leucovorin | Wellcovorin ™ , | Immunex, Corp., |
| (L-Glutamic acid, N[4[[(2-amino-5-formyl- | Leucovorin ™ | Seattle, WA |
| 1,4,5,6,7,8-hexahydro-4oxo-6-pteridinyl) | | |
| methyl]amino]benzoyl], calcium salt (1:1)) | | |
| levamisole HCl | Ergamisol ™ | Janssen Research |
| ((-)-( S)-2,3,5, 6-tetrahydro-6-phenylimidazo | | Foundation, |
| [2,1-b] thiazole monohydrochloride | | Titusville, NJ |
| C₁₁H₁₂N₂S•HCl) | | |
| lomustine | CeeNU ™ | Bristol-Myers |
| (1-(2-chloro-ethyl)-3-cyclohexyl-1- | | Squibb |
| nitrosourea) | | |
| meclorethamine, nitrogen mustard | Mustargen ™ | Merck |
| (2-chloro-N-(2-chloroethyl)-N- | | |
| methylethanamine hydrochloride) | | |
| megestrol acetate | Megace ™ | Bristol-Myers |
| 17α(acetyloxy)-6-methylpregna-4,6-diene- | | Squibb |
| 3,20-dione | | |
| melphalan, L-PAM | Alkeran ™ | GlaxoSmithKline |
| (4-[bis(2-chloroethyl) amino]-L-phenylalanine) | | |
| mercaptopurine, 6-MP | Purinethol ™ | GlaxoSmithKline |
| (1,7-dihydro-6H-purine-6-thione | | |
| monohydrate) | | |
| mesna | Mesnex ™ | Asta Medica |
| (sodium 2-mercaptoethane sulfonate) | | |
| methotrexate | Methotrexate ™ | Lederle |
| (N-[4-[[(2,4-diamino-6- | | Laboratories |
| pteridinyl)methyl]methylamino]benzoyl]-L- | | |
| glutamic acid) | | |
| methoxsalen | Uvadex ™ | Therakos, Inc., Way |
| (9-methoxy-7H-furo[3,2-g][1]-benzopyran-7-one) | | Exton, Pa |
| mitomycin C | Mutamycin ™ | Bristol-Myers |
| | | Squibb |
| mitomycin C | Mitozytrex ™ | SuperGen, Inc., |
| | | Dublin, CA |
| mitotane | Lysodren ™ | Bristol-Myers |
| (1,1-dichloro-2-(o-chlorophenyl)-2-(p- | | Squibb |
| chlorophenyl) ethane) | | |
| mitoxantrone | Novantrone ™ | Immunex |
| (1,4-dihydroxy-5,8-bis[[2-[(- | | Corporation |
| hydroxyethyl)amino]ethyl]amino]-9,10- | | |
| anthracenedione dihydrochloride) | | |
| nandrolone phenpropionate | Duraboln-50 ™ | Organon, Inc., West |
| | | Orange, NJ |
| nofetumomab | Verluma ™ | Boehringer |
| | | Ingelheim Pharma |
| | | KG, Germany |
| oprelvekin | Neumega ™ | Genetics Institute, |
| (IL-11) | | Inc., Alexandria, |
| | | VA |
| pamidronate | Aredia ™ | Novartis |
| (phosphonic acid (3-amino-1- | | |
| hydroxypropylidene) bis-, disodium salt, | | |
| pentahydrate, (APD)) | | |
| pegademase | Adagen ™ | Enzon |
| ((monomethoxypolyethylene glycol | (Pegademase | Pharmaceuticals, |
| succinimidyl) 11-17-adenosine deaminase) | Bovine) | Inc., Bridgewater, |
| | | NJ |
| pegaspargase | Oncaspar ™ | Enzon |
| (monomethoxypolyethylene glycol | | |
| succinimidyl L-asparaginase) | | |
| pegfilgrastim | Neulasta ™ | Amgen, Inc |
| (covalent conjugate of recombinant methionyl | | |
| human G-CSF (Filgrastim) and | | |
| monomethoxypolyethylene glycol) | | |
| pentostatin | Nipent ™ | Parke-Davis |
| | | Pharmaceutical Co., |
| | | Rockville, MD |
| pipobroman | Vercyte ™ | Abbott |
| | | Laboratories, |
| | | Abbott Park, IL |
| plicamycin, mithramycin | Mithracin ™ | Pfizer, Inc., NY, |
| (antibiotic produced by Streptomyces plicatus) | | NY |
| quinacrine | Atabrine ™ | Abbott Labs |
| (6-chloro-9-(1-methyl-4-diethyl-amine) | | |
| butylamino-2-methoxyacridine) | | |
| rasburicase | Elitek ™ | Sanofi-Synthelabo, |
| (recombinant peptide) | | Inc., |
| sargramostim | Prokine ™ | Immunex Corp |
| (recombinant peptide) | | |
| streptozocin | Zanosar ™ | Pharmacia & |
| (streptozocin 2-deoxy-2- | | Upjohn Company |
| [[(methylnitrosoamino)carbonyl]amino]- | | |
| a(and b)-D-glucopyranose and 220 mg citric | | |
| acid anhydrous) | | |
| talc | Sclerosol ™ | Bryan, Corp., |
| (Mg₃Si₄O₁₀ (OH)₂) | | Woburn, MA |
| temozolomide | Temodar ™ | Schering |
| (3,4-dihydro-3-methyl-4-oxoimidazo[5,1-d]- | | |
| as-tetrazine-8-carboxamide) | | |
| teniposide, VM-26 | Vumon ™ | Bristol-Myers |
| (4′-demethylepipodophyllotoxin 9-[4,6-0-(R)- | | Squibb |
| 2-thenylidene-(beta)-D-glucopyranoside]) | | |
| testolactone | Teslac ™ | Bristol-Myers |
| (13-hydroxy-3-oxo-13,17-secoandrosta-1,4- | | Squibb |
| dien-17-oic acid [dgr]-lactone) | | |
| thioguanine, 6-TG | Thioguanine ™ | GlaxoSmithKline |
| (2-amino-1,7-dihydro-6H-purine-6-thione) | | |
| thiotepa | Thioplex ™ | Immunex |
| (Aziridine, 1,1′,1″-phosphinothioylidynetris-, | | Corporation |
| or Tris (1-aziridinyl) phosphine sulfide) | | |
| topotecan HCl | Hycamtin ™ | GlaxoSmithKline |
| ((S)-10-[(dimethylamino) methyl]-4-ethyl-4,9- | | |
| dihydroxy-1H-pyrano[3′, 4′: 6,7] indolizino | | |
| [1,2-b] quinoline-3,14-(4H,12H)-dione | | |
| monohydrochloride) | | |
| toremifene | Fareston ™ | Roberts |
| (2-(p-[(Z)-4-chloro-1,2-diphenyl-1-butenyl]- | | Pharmaceutical |
| phenoxy)-N,N-dimethylethylamine citrate (1:1)) | | Corp., Eatontown, |
| | | NJ |
| tositumomab, I 131 tositumomab | Bexxar ™ | Corixa Corp., |
| (recombinant murine immunotherapeutic | | Seattle, WA |
| monoclonal IgG₂ₐ lambda anti-CD20 antibody | | |
| (I 131 is a radioimmunotherapeutic antibody)) | | |
| tretinoin, ATRA | Vesanoid ™ | Roche |
| (all-trans retinoic acid) | | |
| uracil mustard | Uracil Mustard | Roberts Labs |
| | Capsules ™ | |
| valrubicin, N-trifluoroacetyladriamycin-14- | Valstar ™ | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| zoledronate, zoledronic acid | Zometa ™ | Novartis |
| ((1-Hydroxy-2-imidazol-1-yl-phosphonoethyl) | | |
| phosphonic acid monohydrate) | | |

23. Other Chemotherapeutic Agents

Additional drugs that may be co-administered with compounds of the present invention include metformin, insulin, 2-deoxyglucose, sulfonylureas, anti-diabetic agents generally, mitochondrial oxidative-phoshorylation uncoupling agents, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), mTOR inhibitors, or agents that influence cancer metabolism.

24. Drug Cocktails

Chemotherapy agents can include cocktails of two or more chemotherapy drugs mentioned above. In several embodiments, a chemotherapy agent is a cocktail that includes two or more alkylating agents, platinums, anti-metabolites, anthracyclines, taxanes, camptothecins, nitrosoureas, EGFR inhibitors, antibiotics, HER2/neu inhibitors, angiogenesis inhibitors, kinase inhibitors, proteaosome inhibitors, immunotherapies, hormone therapies, photodynamic therapies, cancer vaccines, sphingolipid modulators, oligomers or combinations thereof

In one embodiment, the chemotherapy agent is a cocktail that includes an immunotherapy, an alkylating agent, an anthracycline, a camptothecin and prednisone. In other embodiments, the chemotherapy agent is a cocktail that includes rituximab, an alkylating agent, an anthracycline, a camptothecin and prednisone. In other embodiments, the chemotherapy agent is a cocktail that includes rituximab, cyclophosphamide, an anthracycline, a camptothecin and prednisone. In still other embodiments, the chemotherapy agent is a cocktail that includes rituximab, cyclophosphamide, doxorubicin, vincristine and prednisone (e.g., R—CHOP). In other embodiments, combination chemotherapeutic regimens may include, but are not limited to ABVD, AC, BEACOPP, BEP, CA, CAF, CAPDX, CAV, CBV, ChIVPP/EVA, CHOP, R—CHOP, COP, CVP, CMF, COPP, CTD, CVAD, Hyper-CVAD, DICE, DT-PACE, EC, ECF, EP, EPOCH, FEC, FL, FOLFIRI, FOLFIRINOX, FOLFOX, ICE, R-ICE, IFL, m-BACOD, MACOP-B, MOPP, MVAC, PCV, POMP, Pro-MACE-MOPP, Pro-MACE CytaBOM, R-FCM, Stanford V, TCH, Thal/Dex, TIP, EE-4a, DD-4a, VAC, VAD, VAMP, Regimen I, VAPEC-B, and VIP or combination including one or more of the following agents: lenalidomide, ofatumumab, obinutuzumab, R05072759, GA101, RG7159, idelalisib, GS-1101, CAL-101, bortezomib, everolimus, ibrutinib, panobinostat, alisertib, brentuximab or vorinostat.

In another embodiment, the chemotherapy agent is a cocktail that includes doxorubicin, ifosfamide and mesna.

In other embodiments, the chemotherapy agent is a cocktail that includes an anti-metabolite and a taxane. For example, the chemotherapy agent includes gemcitabine and taxotere.

In other embodiments, the chemotherapy agent is a cocktail that includes dacarbazine, mitomycin, doxorubicin and cisplatin.

In other embodiments, the chemotherapy agent is a cocktail that includes doxorubicin and dacarbazine.

In alternative embodiments, the chemotherapy agent is a cocktail that includes an alkylating agent, a camptothecins, an anthracycline and dacarbazine. In other examples, the chemotherapy agent includes cyclophosphamide, vincristine, doxorubicin and dacarbazine.

In still other embodiments, the chemotherapy agent is a cocktail that includes an alkylating agent, methotrexate, an anti-metabolite and one or more anthracyclines. For example, the chemotherapy agent includes 5-fluorouracil, methotrexate, cyclophosphamide, doxorubicin and epirubicin.

In yet other embodiments, the chemotherapy agent is a cocktail that includes a taxane and prednisone or estramustine. For example, the chemotherapy agent can include docetaxel combined with prednisone or estramustine.

In still yet another embodiment, the chemotherapy agent includes an anthracycline and prednisone. For example, the chemotherapy agent can include mitoxantrone and prednisone.

In other embodiments, the chemotherapy agent includes a rapamycin macrolide and a kinase inhibitor. The kinase inhibitors can be EGFR, Her2/neu, VEGF, Aurora kinase, SRC/Abl kinase, Bruton's tyrosine kinase, PI3 kinase, and/or MEK inhibitors.

In another embodiment the chemotherapy agent includes two or more sphingolipid modulators.

In still another embodiment the chemotherapy agent includes an oligomer, such as Genasense® and one or more alkylating agents, platinums, anti-metabolites, anthracyclines, taxanes, camptothecins, nitrosoureas, EGFR inhibitors, antibiotics, HER2/neu inhibitors, angiogenesis inhibitors, kinase inhibitors, proteaosome inhibitors, immunotherapies, hormone therapies, photodynamic therapies, cancer vaccines, sphingolipid modulators, PARP inhibitors or combinations thereof

Moreover, the chemotherapy drug or drugs composing the chemotherapy agent can be administered in combination therapies with other agents, or they may be administered sequentially or concurrently to the patient.

b. Radiation Therapy

In several embodiments of the present invention, radiation therapy is administered in addition to the administration of an oligonucleotide compound. Radiation therapy includes both external and internal radiation therapies.

1. External Radiation Therapy

External radiation therapies include directing high-energy rays (e.g., x-rays, gamma rays, and the like) or particles (alpha particles, beta particles, protons, neutrons and the like) at the cancer and the normal tissue surrounding it. The radiation is produced outside the patient's body in a machine called a linear accelerator. External radiation therapies can be combined with chemotherapies, surgery or oligonucleotide compounds.

2. Internal Radiation Therapy

Internal radiation therapies include placing the source of the high-energy rays inside the body, as close as possible to the cancer cells. Internal radiation therapies can be combined with external radiation therapies, chemotherapies or surgery.

Radiation therapy can be administered with chemotherapy simultaneously, concurrently, or separately. Moreover radiation therapy can be administered with surgery simultaneously, concurrently, or separately.

c. Surgery

In alternative embodiments, of the present invention, surgery is used to remove cancerous tissue from a patient. Cancerous tissue can be excised from a patient using any suitable surgical procedure including, for example, laparoscopy, scalpel, laser, scissors and the like. In several embodiments, surgery is combined with chemotherapy. In other embodiments, surgery is combined with radiation therapy. In still other embodiments, surgery is combined with both chemotherapy and radiation therapy.

### IV. Pharmaceutical Compositions

In one aspect of the present invention, a pharmaceutical composition comprises one or more oligonucleotide compounds and a chemotherapy agent. For example, a pharmaceutical composition comprises an oligonucleotide compound having SEQ. ID NO.1250, 1251, 1252, or 1253; and one or more of an alkylating agent, a platinum, an anti-metabolite, an anthracycline, a taxane, a camptothecins, a nitrosourea, an EGFR inhibitor, an antibiotic, a HER2/neu inhibitor, an angiogenesis inhibitor, a proteaosome inhibitor, an immunotherapy, a hormone therapy, a photodynamic therapy, a cancer vaccine, a PARP inhibitor, a cell proliferation inhibitor, other chemotherapy agents such as those illustrated in Table 1, or combinations thereof

In one embodiment, the pharmaceutical composition comprises an oligonucleotide compound and a chemotherapy agent including a dacarbazine, a B-RAF V600E inhibitor, or an antibody that binds to the cytotoxic T lymphocyte-associated antigen 4 (CTLA-4) or combinations thereof. The B-raf inhibitor may be vemurafenib. The CTLA-4 antibody may be ipilimumab.

The pharmaceutical composition may further comprise an immunotherapy, an alkylating agent, an anthracycline, a camptothecin and prednisone. For example, the pharmaceutical composition comprises one or more oligonucleotide compounds comprising SEQ ID NOs 2-281, 283-461, 463-935, 937-1080, 1082-1248, 1250-1254 and 1267-1477, and complements thereof; and a chemotherapy agent including an immunotherapy, an alkylating agent, an anthracycline, a camptothecin, and prednisone. In other embodiments, the pharmaceutical composition comprises an oligonucleotide compound and a chemotherapy agent that includes rituximab, cyclophosphamide, an anthracycline, a camptothecin and prednisone. In still other embodiments, the pharmaceutical composition comprises an oligonucleotide and a chemotherapy agent including rituximab, cyclophosphamide, doxorubicin, vincristine and prednisone (e.g., R—CHOPS). In some embodiments, the pharmaceutical composition may comprise, for example, an oligonucleotide compound and bendamustine. In other embodiments, the pharmaceutical composition may comprise an oligonucleotide compound and fludarabine, cyclophosphamine, and, optionally, rituximab (FCR).

Pharmaceutical compositions of the present invention can optionally include medicaments such as anesthesia, nutritional supplements (e.g., vitamins, minerals, protein and the like), chromophores, combinations thereof, and the like.

A. Oligonucleotide Delivery

The oligonucleotide compounds of the present invention may be delivered using any suitable method. In some embodiments, naked DNA is administered. In other embodiments, lipofection is utilized for the delivery of nucleic acids to a subject. In still further embodiments, oligonucleotides are modified with phosphothiolates for delivery (see e.g., U.S. Pat. No. 6,169,177, herein incorporated by reference).

In some embodiments, oligonucleotides are sequestered in lipids (e.g., liposomes or micelles) to aid in delivery (See e.g., U.S. Pat. Nos. 6,458,382, 6,429,200; U.S. Patent Publications 2003/0099697, 2004/0120997, 2004/0131666, 2005/0164963, and International Publication WO 06/048329, each of which is herein incorporated by reference).

As used herein, “liposome” refers to one or more lipids forming a complex, usually surrounded by an aqueous solution. Liposomes are generally spherical structures comprising lipids, such as phospholipids, steroids, fatty acids, and are lipid bilayer type structures, and can include unilamellar vesicles, multilamellar structures, and amorphous lipid vesicles. Generally, liposomes are completely closed lipid bilayer membranes containing an entrapped aqueous volume. The liposomes may be unilamellar vesicles (possessing a single bilayer membrane) or multilamellar (onion-like structures characterized by multiple membrane bilayers, each separated from the next by an aqueous layer). Liposomes of the present invention may also include a DNAi oligonucleotide as defined below, either bound to the liposomes or sequestered in or on the liposomes. The molecules include, but are not limited to, DNAi oligonucleotides and/or other agents used to treat diseases such as cancer.

As used herein, “sequestered”, “sequestering”, or “sequester” refers to encapsulation, incorporation, or association of a drug, molecule, compound, including a DNAi oligonucleotide, with the lipids of a liposome. The molecule may be associated with the lipid bilayer or present in the aqueous interior of the liposome or both. “Sequestered” includes encapsulation in the aqueous core of the liposome. It also encompasses situations in which part or all of the molecule is located in the aqueous core of the liposome and part outside of the liposome in the aqueous phase of the liposomal suspension, where part of the molecule is located in the aqueous core of the liposome and part in the lipid portion of the liposome, or part sticking out of the liposomal exterior, where molecules are partially or totally embedded in the lipid portion of the liposome, and includes molecules associated with the liposomes, with all or part of the molecule associated with the exterior of the liposome.

Particularly, after a systemic application, the oligonucleotide and/or other agents must be stably sequestered in the liposomes until eventual uptake in the target tissue or cells. Accordingly, the guidelines for liposomal formulations of the FDA regulate specific preclinical tests for liposomal drugs (http://www.fda.gov/cder/guidance/2191dft.pdf). After injection of liposomes into the blood stream, serum components interact with the liposomes, which can lead to permeabilization of the liposomes. However, release of a drug or molecule that is encapsulated in a liposome depends on molecular dimensions of the drug or molecule. Consequently, a plasmid of thousands of base pairs is released much more slowly than smaller oligonucleotides or other small molecules. For liposomal delivery of drugs or molecules, it is ideal that the release of the drug during circulation of the liposomes in the bloodstream be as low as possible.

1. Amphoteric Liposomes

In some embodiments, liposomes used for delivery may be amphoteric liposomes, such as those described in US 2009/0220584, incorporated herein by reference. Amphoteric liposomes are a class of liposomes having anionic or neutral charge at about pH 7.5 and cationic charge at pH 4. Lipid components of amphoteric liposomes may be themselves amphoteric, and/or may consist of a mixture of anionic, cationic, and in some cases, neutral species, such that the liposome is amphoteric.

As used herein, an “amphoteric liposome” is a liposome with an amphoteric character, as defined below.

As used herein, sequestered, sequestering, or sequester refers to encapsulation, incorporation, or association of a drug, molecule, compound, including a DNAi oligonucleotide, with the lipids of a liposome. The molecule may be associated with the lipid bilayer or present in the aqueous interior of the liposome or both. “Sequestered” includes encapsulation in the aqueous core of the liposome. It also encompasses situations in which part or all of the molecule is located in the aqueous core of the liposome and part outside of the liposome in the aqueous phase of the liposomal suspension, where part of the molecule is located in the aqueous core of the liposome and part in the lipid portion of the liposome, or part sticking out of the liposomal exterior, where molecules are partially or totally embedded in the lipid portion of the liposome, and includes molecules associated with the liposomes, with all or part of the molecule associated with the exterior of the liposome.

As used herein, “polydispersity index” is a measure of the heterogeneity of the particle dispersion (heterogeneity of the diameter of liposomes in a mixture) of the liposomes. A polydispersity index can range from 0.0 (homogeneous) to 1.0 (heterogeneous) for the size distribution of liposomal formulations.

The amphoteric liposomes include one or more amphoteric lipids or alternatively a mix of lipid components with amphoteric properties. Suitable amphoteric lipids are disclosed in PCT International Publication Number WO02/066489 as well as in PCT International Publication Number WO03/070735, the contents of both of which are incorporated herein by reference. Alternatively, the lipid phase may be formulated using pH-responsive anionic and/or cationic components, as disclosed in PCT International Publication Number WO02/066012, the contents of which are incorporated by reference herein. Cationic lipids sensitive to pH are disclosed in PCT International Publication Numbers WO02/066489 and WO03/070220, in Budker, et al. 1996, Nat. Biotechnol., 14(6):760-4, and in U.S. Pat. No. 6,258,792 the contents of which are incorporated by reference herein, and can be used in combination with constitutively charged anionic lipids or with anionic lipids that are sensitive to pH. Conversely, the cationic charge may also be introduced from constitutively charged lipids that are known to those skilled in the art in combination with a pH sensitive anionic lipid. (See also PCT International Publication Numbers WO05/094783, WO03/070735, WO04/00928, WO06/48329, WO06/053646, WO06/002991 and U.S. Patent publications 2003/0099697, 2005/0164963, 2004/0120997, 2006/159737, 2006/0216343, each of which is also incorporated in its entirety by reference.)

Amphoteric liposomes of the present invention include (1) amphoteric lipids or a mixture of lipid components with amphoteric properties, (2) neutral lipids, (3) one or more DNAi oligonucleotides, (4) a cryoprotectant and/or lyoprotectant, and (5) a spray-drying cryoprotectant. In addition, the DNAi-liposomes have a defined size distribution and polydispersity index.

As used herein, “amphoter” or “amphoteric” character refers to a structure, being a single substance (e.g., a compound) or a mixture of substances (e.g., a mixture of two or more compounds) or a supramolecular complex (e.g., a liposome) comprising charged groups of both anionic and cationic character wherein

(i) at least one of the charged groups has a pK between 4 and 8,

(ii) the cationic charge prevails at pH 4 and

(iii) the anionic charge prevails at pH 8,

resulting in an isoelectric point of neutral net charge between pH 4 and pH 8 Amphoteric character by that definition is different from zwitterionic character, as zwitterions do not have a pK in the range mentioned above. Consequently, zwitterions are essentially neutrally charged over a range of pH values. Phosphatidylcholine or phosphatidylethanolamines are neutral lipids with zwitterionic character.

As used herein, “Amphoter I Lipid Pairs” refers to lipid pairs containing a stable cation and a chargeable anion. Examples include without limitation DDAB/CHEMS, DOTAP/CHEMS and DOTAP/DOPS. In some aspects, the ratio of the percent of cationic lipids to anionic lipids is lower than 1.

As used herein, “Amphoter II Lipid Pairs” refers to lipid pairs containing a chargeable cation and a chargeable anion. Examples include without limitation Mo-Chol/CHEMS, DPIM/CHEMS or DPIM/DG-Succ. In some aspects, the ratio of the percent of cationic lipids to anionic lipids is between about 5 and 0.2.

As used herein, “Amphoter III Lipid Pairs” refers to lipid pairs containing a chargeable cation and stable anion. Examples include without limitation Mo-Chol/DOPG or Mo-Chol/Chol-SO₄. In one embodiment, the ratio of the percent of cationic lipids to anionic lipids is higher than 1.

Abbreviations for lipids refer primarily to standard use in the literature and are included here as a helpful reference:
DMPC Dimyristoylphosphatidylcholine
DPPC Dipalmitoylphosphatidylcholine
DSPC Distearoylphosphatidylcholine
POPC Palmitoyl-oleoylphosphatidylcholine
OPPC 1-oleoyl-2-palmitoyl-sn-glycero-3-phosphocholine
DOPC Dioleoylphosphatidylcholine
DOPE Dioleoylphosphatidylethanolamine
DMPE Dimyristoylphosphatidylethanolamine
DPPE Dipalmitoylphosphatidylethanolamine
DOPG Dioleoylphosphatidylglycerol
POPG Palmitoyl-oleoylphosphatidylglycerol
DMPG Dimyristoylphosphatidylglycerol
DPPG Dipalmitoylphosphatidylglycerol
DLPG Dilaurylphosphatidylglycerol
DSPG Distearoylphosphatidylglycerol
DMPS Dimyristoylphosphatidylserine
DPPS Dipalmitoylphosphatidylserine
DOPS Dioleoylphosphatidylserine
POPS Palmitoyl-oleoylphosphatidylserine
DMPA Dimyristoylphosphatidic acid
DPPA Dipalmitoylphosphatidic acid
DSPA Distearoylphosphatidic acid
DLPA Dilaurylphosphatidic acid
DOPA Dioleoylphosphatidic acid
POPA Palmitoyl-oleoylphosphatidic acid
CHEMS Cholesterolhemisuccinate
DC-Chol 3-β-[N—(N′,N′-dimethylethane)carbamoyl]cholesterol
Cet-P Cetylphosphate
DODAP (1,2)-dioleoyloxypropyl)-N,N-dimethylammonium chloride
DOEPC 1,2-dioleoyl-sn-glycero-3-ethylphosphocholine
DAC-Chol 3-β-[N—(N,N′-dimethylethane) carbamoyl]cholesterol
TC-Chol 3-β-[N—(N′,N′,N′-trimethylaminoethane) carbamoyl]cholesterol
DOTMA (1,2-dioleyloxypropyl)-N,N,N-trimethylammoniumchloride) (Lipofectin®)
DOGS ((C18)2GlySper3+) N,N-dioctadecylamido-glycyl-spermine (Transfectam®)
CTAB Cetyl-trimethylammoniumbromide
CPyC Cetyl-pyridiniumchloride
DOTAP (1,2-dioleoyloxypropyl)-N,N,N-trimethylammonium salt
DMTAP (1,2-dimyristoyloxypropyl)-N,N,N-trimethylammonium salt
DPTAP (1,2-dipalmitoyloxypropyl)-N,N,N-trimethylammonium salt
DOTMA (1,2-dioleyloxypropyl)-N,N,N-trimethylammonium chloride)
DORIE (1,2-dioleyloxypropyl)-3 dimethylhydroxyethyl ammoniumbromide)
DDAB Dimethyldioctadecylammonium bromide
DPIM 4-(2,3-bis-palmitoyloxy-propyl)-1-methyl-1H-imidazole
CHIM Histaminyl-Cholesterolcarbamate
MoChol 4-(2-Aminoethyl)-Morpholino-Cholesterolhemisuccinate
HisChol Histaminyl-Cholesterolhemisuccinate
HCChol Nα-Histidinyl-Cholesterolcarbamate
HistChol Nα-Histidinyl-Cholesterol-hemisuccinate
AC Acylcarnosine, Stearyl- & Palmitoylcarnosine
HistDG 1,2-Dipalmitoylglycerol-hemisuccinat-N-Histidinyl-hemisuccinate; and Distearoyl-, Dimyristoyl-, Dioleoyl- or palmitoyl-oleoyl derivatives
IsoHistSuccDG 1,2-ipalmitoylglycerol-O-Histidinyl-Nα-hemisuccinate, and Distearoyl-, Dimyristoyl, Dioleoyl or palmitoyl-oleoyl derivatives
DGSucc 1,2-Dipalmitoyglycerol-3-hemisuccinate & Distearoyl-, dimyristoyl- Dioleoyl or palmitoyl-oleoylderivatives
EDTA-Chol cholesterol ester of ethylenediaminetetraacetic acid
Hist-PS Nα-histidinyl-phosphatidylserine
BGSC bisguanidinium-spermidine-cholesterol
BGTC bisguanidinium-tren-cholesterol
DOSPER (1,3-dioleoyloxy-2-(6-carboxy-spermyl)-propylarnide
DOSC (1,2-dioleoyl-3-succinyl-sn-glyceryl choline ester)
DOGSDO (1,2-dioleoyl-sn-glycero-3-succinyl-2-hydroxyethyl disulfide ornithine)
DOGSucc 1,2-Dioleoylglycerol-3-hemisucinate
POGSucc Palimtolyl-oleoylglycerol-oleoyl-3-hemisuccinate
DMGSucc 1,2-Dimyristoylglycerol-3-hemisuccinate
DPGSucc 1,2-Dipalmitoylglycerol-3-hemisuccinate

The following structures provide non-limiting examples of lipids that are suitable for use in the compositions in accordance with the present invention. The membrane anchors of the lipids are shown exemplarily and serve only to illustrate the lipids of the invention and are not intended to limit the same.

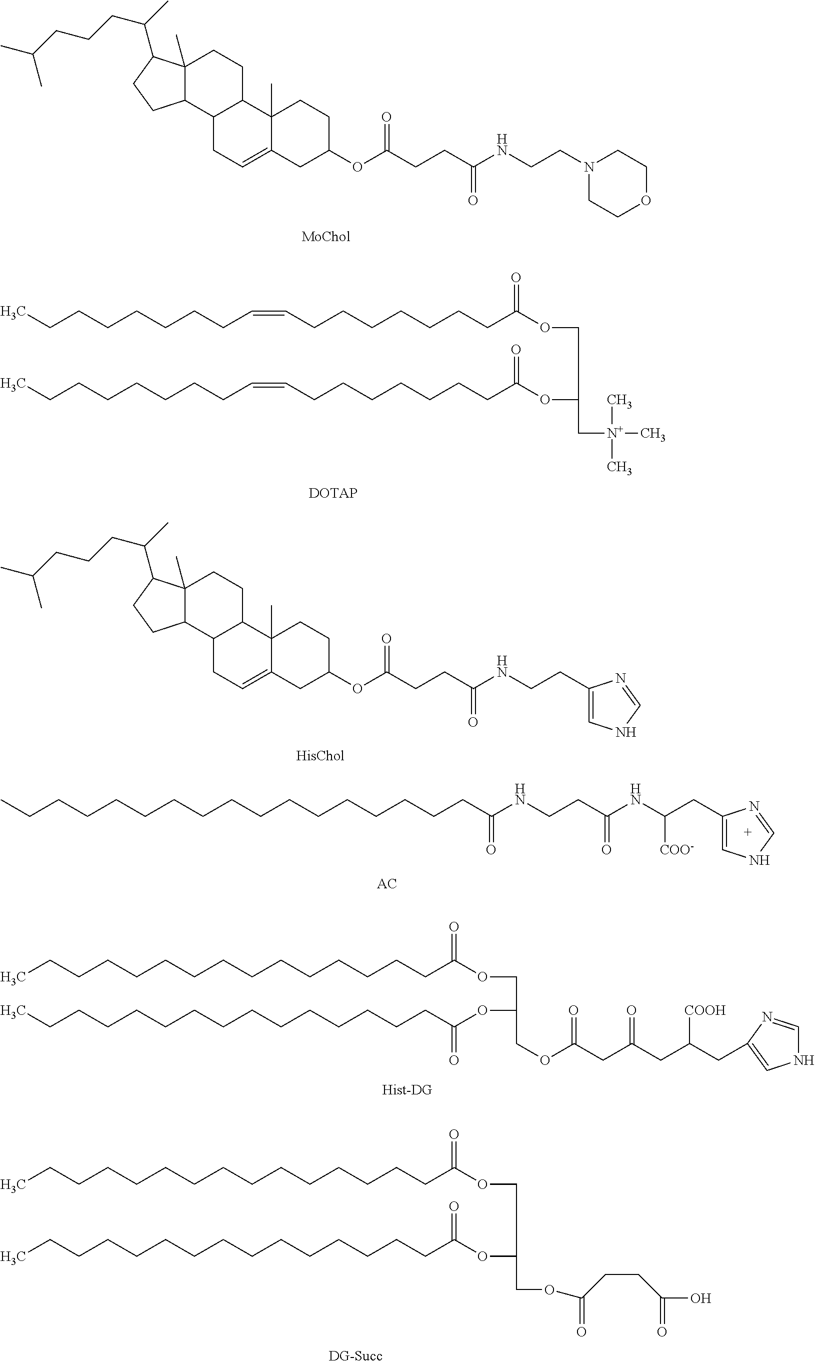

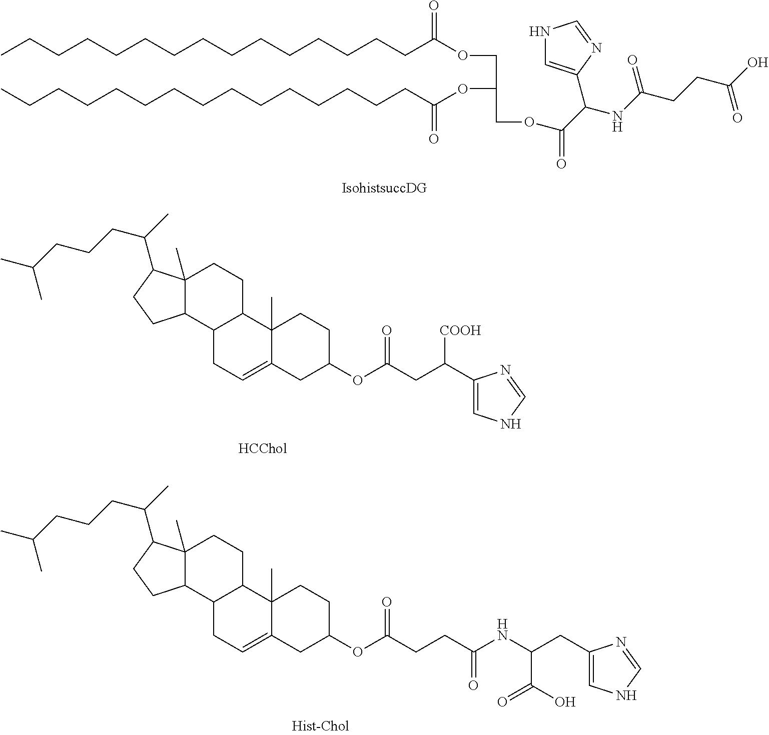 [SMILES: CCCCCCCCCCCCCCCC(=O)OCC(COC(=O)C(NC(=O)CCC(=O)O)c1c[nH]cn1)OC(=O)CCCCCCCCCCCCCCC ; CC(C)CCCC(C)C1CCC2C3CC=C4CC(OC(=O)CC(C(=O)O)c5c[nH]cn5)CCC4(C)C3CCC12C ; CC(C)CCCC(C)C1CCC2C3CC=C4CC(OC(=O)CCC(=O)NC(Cc5c[nH]cn5)C(=O)O)CCC4(C)C3CCC12C]

Amphoteric lipids are disclosed in PCT International Publication Numbers WO02/066489 and WO03/070735, the contents of both of which are incorporated herein by reference. The overall molecule assumes its pH-dependent charge characteristics by the simultaneous presence of cationic and anionic groups in the “amphoteric substance” molecule portion. More specifically, an amphoteric substance is characterized by the fact that the sum of its charge components will be precisely zero at a particular pH value. This point is referred to as isoelectric point (IP). Above the IP the compound has a negative charge, and below the IP it is to be regarded as a positive cation, the IP of the amphoteric lipids according to the invention ranging between 4.5 and 8.5.

The overall charge of the molecule at a particular pH value of the medium can be calculated as follows:

z=Σnᵢ×((qᵢ−1)+(10^{(pK-pH)}/(1+10^{(pK-pH)}))
qᵢ: absolute charge of the ionic group below the pK thereof (e.g. carboxyl=0, single-nitrogen base=1, di-esterified phosphate group=−1)
nᵢ number of such groups in the molecule.

For example, a compound is formed by coupling the amino group of histidine to cholesterol hemisuccinate. At a neutral pH value of 7, the product has a negative charge because the carboxyl function which is present therein is in its fully dissociated form, and the imidazole function only has low charge. At an acid pH value of about 4, the situation is reversed: the carboxyl function now is largely discharged, while the imidazole group is essentially fully protonated, and the overall charge of the molecule therefore is positive.

In one embodiment, the amphoteric lipid is selected from the group consisting of HistChol, HistDG, isoHistSuccDG, Acylcarnosine and HCChol. In another embodiment, the amphoteric lipid is HistChol.

Amphoteric lipids can include, without limitation, derivatives of cationic lipids which include an anionic substituent Amphoteric lipids include, without limitation, the compounds having the structure of the formula:

Z—X-W1-Y-W2-HET

wherein:

Z is a sterol or an aliphatic; Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesteril, dihydrocholesterol, 19-hydroxycholesterol, 5α-cholest-7-en-3β-ol, 7-hydroxycholesterol, epocholesterol, ergosterol dehydroergosterol, and derivatives thereof;

Each W1 is independently an unsubstituted aliphatic;

Each W2 is independently an aliphatic optionally substituted with HO(O)C-aliphatic-amino or carboxy;

Each X and Y is independently absent, —(C═O)—O—, —(C═O)—NH—, —(C═O)—S—, —O—, —NH—, —S—, —CH═N—, —O—(O═C)—, —S—(O═C)—, —NH—(O═C)—, —N═CH—, and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl, or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

In other embodiments, amphoteric lipids include, without limitation, the compounds having the structure of the formula:

Z—X-W1-Y-W2-HET

wherein:

Z is a structure according to the general formula

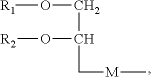

wherein R1 and R2 are independently C₈-C₃₀ alkyl or acyl chains with 0, 1 or 2 ethylenically unsaturated bonds and M is selected from the group consisting of —O—(C═O); —NH—(C═O)—; —S—(C═O)—; —O—; —NH—; —S—; —N═CH—; —(O═C)—O—; —S—(O═C)—; —NH—(O═C)—, —N═CH—, —S—S—; and

Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesteril, dihydrocholesterol, 19-hydroxycholesterol, 5αcholest-7-en-3β-ol, 7-hydroxycholesterol, epicholesterol, ergosterol dehydroergosterol, and derivatives thereof;

Each W1 is independently an unsubstituted aliphatic with up to 8 carbon atoms;

Each W2 is independently an aliphatic, carboxylic acid with up to 8 carbon atoms and 0, 1, or 2 ethyleneically unsaturated bonds;

X is absent and Y is —(C═O)—O—; —(C═O)—NH—; —NH—(C═O)—O—; —O—; —NH—; —CH═N—; —O—(O═C)—; —S—; —(O═C)—; —NH—(O═C)—; —O—(O═C)—NH—, —N═CH— and/or —S—S—; and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl, or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

Alternatively, the lipid phase can be formulated using pH-responsive anionic and/or cationic components, as disclosed in PCT International Publication Number WO02/066012, the contents of which are incorporated by reference herein. Cationic lipids sensitive to pH are disclosed in PCT International Publication Numbers WO02/066489 and WO03/070220, in Budker, et al. (1996), Nat Biotechnol. 14(6):760-4, and in U.S. Pat. No. 6,258,792, the contents of all of which are incorporated by reference herein. Alternatively, the cationic charge may be introduced from constitutively charged lipids known to those skilled in the art in combination with a pH sensitive anionic lipid. Combinations of constitutively (e.g., stable charge over a specific pH range such as a pH between about 4 and 9) charged anionic and cationic lipids, e.g. DOTAP and DPPG are not preferred. Thus, in some embodiments of the invention, the mixture of lipid components may comprise (i) a stable cationic lipid and a chargeable anionic lipid, (ii) a chargeable cationic lipid and chargeable anionic lipid or (iii) a stable anionic lipid and a chargeable cationic lipid.

The charged groups can be divided into the following 4 groups.

(1) Strongly (e.g., constitutively charged) cationic, pKa>9, net positive charge: on the basis of their chemical nature, these are, for example, ammonium, amidinium, guanidium or pyridinium groups or timely, secondary or tertiary amino functions.

(2) Weakly cationic, pKa<9, net positive charge: on the basis of their chemical nature, these are, in particular, nitrogen bases such as piperazines, imidazoles and morpholines, purines or pyrimidines. Such molecular fragments, which occur in biological systems, are, for example, 4-imidazoles (histamine), 2-, 6-, or 9-purines (adenines, guanines, adenosines or guanosines), 1-, 2- or 4-pyrimidines (uracils, thymines, cytosines, uridines, thymidines, cytidines) or also pyridine-3-carboxylic acids (nicotinic esters or amides). Nitrogen bases with preferred pKa values are also formed by substituting nitrogen atoms one or more times with low molecular weight alkene hydroxyls, such as hydroxymethyl or hydroxyethyl groups. For example, aminodihydroxypropanes, triethanolamines, tris-(hydroxymethyl)methylamines, bis-(hydroxymethyl)methylamines, tris-(hydroxyethyl)methylamines, bis-(hydroxyethyl)methylamines or the corresponding substituted ethylamines.

(3) Weakly anionic, pKa>4, net negative charge: on the basis of their chemical nature, these are, in particular, the carboxylic acids. These include the aliphatic, linear or branched mono-, di- or tricarboxylic acids with up to 12 carbon atoms and 0, 1 or 2 ethylenically unsaturated bonds. Carboxylic acids of suitable behavior are also found as substitutes of aromatic systems. Other weakly anionic groups are hydroxyls or thiols, which can dissociate and occur in ascorbic acid, N-substituted alloxane, N-substituted barbituric acid, veronal, phenol or as a thiol group.

(4) Strongly (e.g., constitutively charged) anionic, pKa<4, net negative charge: on the basis of their chemical nature, these are functional groups such as sulfonate or phosphate esters.

The amphoteric liposomes contain variable amounts of such membrane-forming or membrane-based amphiphilic materials, so that they have an amphoteric character. This means that the liposomes can change the sign of the charge completely. The amount of charge carrier of a liposome, present at a given pH of the medium, can be calculated using the following formula:

z=Σnᵢ((qᵢ−1)+10^{(pK-pH)}/(1+^{(pK-pH)}))
in which
qᵢ is the absolute charge of the individual ionic groups below their pK (for example, carboxyl=0, simple nitrogen base=1, phosphate group of the second dissociation step=−1, etc.)
nᵢ is the number of these groups in the liposome.

At the isoelectric point, the net charge of the liposome is 0. Structures with a largely selectable isoelectric point can be produced by mixing anionic and cationic portions.

In one embodiment, cationic components include DPIM, CHIM, DORIE, DDAB, DAC-Chol, TC-Chol, DOTMA, DOGS, (C₁₈)₂Gly⁺ N,N-dioctadecylamido-glycine, CTAB, CPyC, DODAP DMTAP, DPTAP, DOTAP, DC-Chol, MoChol, HisChol and DOEPC. In another embodiment, cationic lipids include DMTAP, DPTAP, DOTAP, DC-Chol, MoChol and HisChol.

The cationic lipids can be compounds having the structure of the formula

L-X-spacer1-Y-spacer2-HET

wherein:

L is a sterol or [aliphatic(C(O)O)—]₂-alkyl-;

Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesteril, dihydrocholesterol, 19-hydroxycholesterol, 5αcholest-7-en-3β-ol, 7-hydroxycholesterol, epocholesterol, ergosterol dehydroergosterol, and derivatives thereof;

Each spacer 1 and spacer 2 is independently an unsubstituted aliphatic;

Each X and Y is independently absent, —(C═O)—O—, —(C═O)—NH—, —(C═O)—S—, —O—, —NH—, —S—, —CH═N—, —O—(O═C)—, —S—(O═C)—, —NH—(O═C)—, —N═CH—, and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

In another embodiment, pH sensitive cationic lipids can be compounds having the structure of the formula

L-X-spacer1-Y-spacer2-HET

wherein:

L is a structure according to the general formula

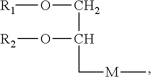

wherein R1 and R2 are independently C₈-C₃₀ alkyl or acyl chains with 0, 1 or 2 ethylenically unsaturated bonds and M is absent, —O—(C═O); —NH—(C═O)—; —S—(C═O)—; —O—; —NH—; —S—; —N═CH—; —(O═C)—O—; —S—(O═C)—; —NH—(O═C)—; —N═CH—, —S—S—; and

Sterol is selected from the group consisting of cholesterol, sitosterol, campesterol, desmosterol, fucosterol, 22-ketosterol, 20-hydroxysterol, sigmasterol, 22-hydroxycholesterol, 25 hydroxycholesterol, lanosterol, 7-dehydrocholesterol, dihydrocholesterol, 19-hydroxycholesterol, 5α-cholest-7-en-3β-ol, 7-hydroxycholesterol, epicholesterol, ergosterol dehydroergosterol, and derivatives thereof;

Each spacer 1 and spacer 2 is independently an unsubstituted aliphatic with 1-8 carbon atoms;

X is absent and Y is absent, —(C═O)—O—; —(C═O)—NH—; —NH—(C═O)—O—; —O—; —NH—; —CH═N—; —O—(O═C)—; —S—; —(O═C)—; —NH—(O═C)—; —O—(O═C)—NH—, —N═CH— and/or —S—S—; and

HET is an amino, an optionally substituted heterocycloaliphatic or an optionally substituted heteroaryl.

In some aspects, the HET is an optionally substituted heterocycloaliphatic including at least one nitrogen ring atom, or an optionally substituted heteroaryl including at least one nitrogen ring atom. In other aspects, the HET is morpholinyl, piperidinyl, piperazinlyl, pyrimidinyl or pyridinyl. In another aspect, the cationic lipid has the structure Sterol-X-spacer1-Y-spacer2-morpholinyl or Sterol-X-spacer1-Y-spacer2-imidazolyl. In still further aspects, the sterol is cholesterol.

The above compounds can be synthesized using syntheses of 1 or more steps, and can be prepared by one skilled in the art.

The amphoteric mixtures further comprise anionic lipids, either constitutively or conditionally charged in response to pH, and such lipids are also known to those skilled in the art. In one embodiment, lipids for use with the invention include DOGSucc, POGSucc, DMGSucc, DPGSucc, DMPS, DPPS, DOPS, POPS, DMPG, DPPG, DOPG, POPG, DMPA, DPPA, DOPA, POPA, CHEMS and CetylP. In another embodiment, anionic lipids include DOGSucc, DMGSucc, DMPG, DPPG, DOPG, POPG, DMPA, DPPA, DOPA, POPA, CHEMS and CetylP.

Neutral lipids include any lipid that remains neutrally charged at a pH between about 4 and 9. Neutral lipids include, without limitation, cholesterol, other sterols and derivatives thereof, phospholipids, and combinations thereof. The phospholipids include any one phospholipid or combination of phospholipids capable of forming liposomes. They include phosphatidylcholines, phosphatidylethanolamines, lecithin and fractions thereof, phosphatidic acids, phosphatidylglycerols, phosphatidylinolitols, phosphatidylserines, plasmalogens and sphingomyelins. The phosphatidylcholines include, without limitation, those obtained from egg, soy beans or other plant sources or those that are partially or wholly synthetic or of variable lipid chain length and unsaturation, POPC, OPPC, natural or hydrogenated soy bean PC, natural or hydrogenated egg PC, DMPC, DPPC, DSPC, DOPC and derivatives thereof. In one embodiment, phosphatidylcholines are POPC, non-hydrogenated soy bean PC and non-hydrogenated egg PC. Phosphatidylethanolamines include, without limitation, DOPE, DMPE and DPPE and derivatives thereof. Phosphatidylglycerols include, without limitation, DMPG, DLPG, DPPG, and DSPG. Phosphatidic acids include, without limitation, DSPA, DMPA, DLPA and DPPA.

Sterols include cholesterol derivatives such as 3-hydroxy-5.6-cholestene and related analogs, such as 3-amino-5.6-cholestene and 5,6-cholestene, cholestane, cholestanol and related analogs, such as 3-hydroxy-cholestane; and charged cholesterol derivatives such as cholesteryl-beta-alanine and cholesterol hemisuccinate. Sterols further include MoChol and analogues of MoChol.

In one embodiment neutral lipids include but are not limited to DOPE, POPC, soy bean PC or egg PC and cholesterol.

In some aspects, the invention provides a mixture comprising amphoteric liposomes and a DNAi oligonucleotide. In an embodiment of the first aspect, the amphoteric liposomes have an isoelectric point of between 4 and 8. In a further embodiment, the amphoteric liposomes are negatively charged or neutral at pH 7.4 and positively charged at pH 4.

In some embodiments, the amphoteric liposomes include amphoteric lipids. In a further embodiment, the amphoteric lipids can be HistChol, HistDG, isoHistSucc DG, Acylcarnosine, HCChol or combinations thereof. In another embodiment, the amphoteric liposomes include a mixture of one or more cationic lipids and one or more anionic lipids. In yet another embodiment, the cationic lipids can be DMTAP, DPTAP, DOTAP, DC-Chol, MoChol or HisChol, or combinations thereof, and the anionic lipids can be CHEMS, DGSucc, Cet-P, DMGSucc, DOGSucc, POGSucc, DPGSucc, DG Succ, DMPS, DPPS, DOPS, POPS, DMPG, DPPG, DOPG, POPG, DMPA, DPPA, DOPA, POPA or combinations thereof.

In yet another embodiment, the liposomes also include neutral lipids. In a further embodiment, the neutral lipids include sterols and derivatives thereof. In an even further embodiment, the sterols comprise cholesterol and derivatives thereof. The neutral lipids may also include neutral phospholipids. In one embodiment, the phospholipids include phosphatidylcholines or phosphatidylcholines and phosphoethanolamines. In another embodiment, the phosphatidylcholines are POPC, OPPC, natural or hydrogenated soy bean PC, natural or hydrogenated egg PC, DMPC, DPPC or DOPC and derivatives thereof and the phosphatidylethanolamines are DOPE, DMPE, DPPE or derivatives and combinations thereof. In a further embodiment, the phosphatidylcholine is POPC, OPPC, soy bean PC or egg PC and the phosphatidylethanolamines is DOPE.

In an even further embodiment, the lipids of the amphoteric liposomes include DOPE, POPC, CHEMS and MoChol; POPC, Chol, CHEMS and DOTAP; POPC, Chol, Cet-P and MoChol, or POPC, DOPE, MoChol and DMGSucc.

In another aspect, the amphoteric liposomes of the mixture of the invention can be formed from a lipid phase comprising a mixture of lipid components with amphoteric properties, wherein the total amount of charged lipids in the liposome can vary from 5 mole % to 70 mole %, the total amount of neutral lipids may vary from 20 mole % to 70 mole %, and a DNAi oligonucleotide. In an embodiment of the first aspect, the amphoteric liposomes include 3 to 20 mole % of POPC, 10 to 60 mole % of DOPE, 10 to 60 mole % of MoChol and 10 to 50 mole % of CHEMS. In a further embodiment, the liposomes include POPC, DOPE, MoChol and CHEMS in the molar ratios of POPC/DOPE/MoChol/CHEMS of about 6/24/47/23 or 15/45/20/20. In yet another embodiment, the liposomes include 3 to 20 mole % of POPC, 10 to 40 mole % of DOPE, 15 to 60 mole % of MoChol and 15 to 60 mole % of DMGSucc. In a further embodiment, the liposomes include POPC, DOPE, DMGSucc and MoChol in the molar ratios of POPC/DOPE/DMGSucc/MoChol of about 6/24/47/23 or 6/24/23/47. In still another embodiment, the liposomes include 10 to 50 mole % of POPC, 20 to 60 mole % of Chol, 10 to 40 mole % of CHEMS and 5 to 20 mole % of DOTAP. In a further embodiment, the liposomes include POPC, Chol, CHEMS and DOTAP in the molar ratio of POPC/Chol/CHEMS/DOTAP of about 30/40/20/10. In yet another embodiment the liposomes include 10 to 40 mole % of POPC, 20 to 50 mole % of Chol, 5 to 30 mole % of Cet-P and 10 to 40 mole % of MoChol. Ina further embodiment, the molar ratio of POPC/Chol/Cet-P/MoChol is about 35/35/10/20.

In a third aspect, the DNAi oligonucleotide contained in the amphoteric liposomal mixture comprises a DNAi oligonucleotide that hybridizes to SEQ ID NO:1249 or portions thereof. In another embodiment, the DNAi oligonucleotide can be SEQ ID NO:1250, 1251, 1252, 1253, 1267-1447 or the complement thereof. In yet another embodiment the DNAi oligonucleotide can be SEQ ID NO:1250 or 1251 or the complement thereof.

The amphoteric liposomal mixture of this invention may further include an additional DNAi oligonucleotide, e.g., comprising one of SEQ ID NOs:1250-1253 and 1270-1477, or selected from the group consisting of SEQ ID NOs:2-281, 283-461, 463-935, 937-1080, 1082-1248 and the complements thereof.

In another aspect, the DNAi oligonucleotides contained in the liposomal mixture are between 15 and 35 base pairs in length.

In another aspect, the amphoteric liposome-DNAi oligonucleotide mixture includes the DNAi oligonucleotides SEQ ID NO:1250 or 1251 and amphoteric liposomes comprising POPC, DOPE, MoChol and CHEMS in the molar ratio of POPC/DOPE/MoChol/CHEMS of about 6/24/47/23.

In another aspect, the amphoteric liposome-DNAi oligonucleotide mixture includes the DNAi oligonucleotide, PNT100 (SEQ ID NO:1250 or 1251), and amphoteric liposomes comprising POPC, DOPE, MoChol and CHEMS in the molar ratio of POPC/DOPE/MoChol/CHEMS of about 15/45/20/20.

In another aspect, the amphoteric liposomes of the mixture can include a size between 50 and 500 ηm. In one embodiment, the size is between 80 and 300 ηm and in another embodiment the size is between 90 and 200 ηm.

In another aspect, the amphoteric liposomes may have an isoelectric point between 4 and 8. In an embodiment of the sixth aspect, the amphoteric liposomes may be negatively charged or neutral at pH 7.4 and positively charged at pH 4.

In another aspect, the amphoteric liposomes have a DNAi oligonucleotide concentration of at least about 2 mg/ml at a lipid concentration of 10 to 100 mM or less.

In another aspect, the invention provides a method of preparing amphoteric liposomes containing a DNAi oligonucleotide. In one embodiment, the method includes using an active loading procedure and in another, a passive loading procedure. In a further embodiment, the method produces liposomes using manual extrusion, machine extrusion, homogenization, microfluidization or ethanol injection. In yet another embodiment, the method has an encapsulation efficiency of at least 35%.

In another aspect, the invention provides a method of introducing the DNAi oligonucleotide-amphoteric liposome mixture to cells or an animal. In one embodiment, the method includes administering the mixture to mammal to treat cancer. The administered mixtures can reduce or stop tumor growth in mammals. In another embodiment, the introduction of the mixture results in a reduction of cell proliferation. In another embodiment, the mixture is administered to a cancer cell, a non-human animal or a human. In a further embodiment, the mixture is introduced to an animal at a dosage of between 0.01 mg to 100 mg per kg of body weight. In yet another embodiment, the mixture is introduced to the animal one or more times per day or continuously. In still another embodiment, the mixture is introduced to the animal via topical, pulmonary or parenteral administration or via a medical device. In an even further embodiment, the mixture administered to the animal or cells further includes a chemotherapy agent, and/or a cell targeting component. In yet another embodiment, the mixture may be administered to the mammal in a sequential manner.

In some embodiments, amphoteric liposomes formulations may comprise POPC/DOPE/MoChol/CHEMS at molar ratios of 6/24/47/23, respectively. Such liposomes are cholesterol-rich and negatively-charged. This is unique among lipid delivery systems and contributes to cellular uptake. In some embodiments, oligonucleotides of SEQ ID NO:1251 or 1250 (PNT100) may be sequestered in amphoteric liposomes with this formulation (hereinafter, “PNT2258”).

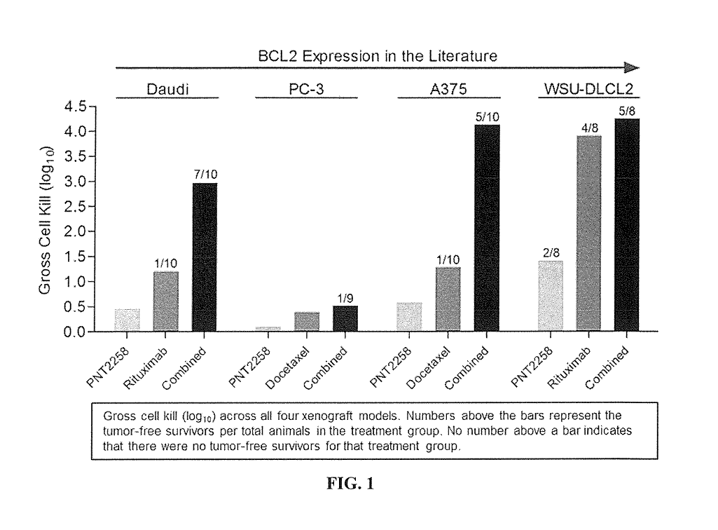

PNT2258, is an innovative therapeutic that is expected to address unmet medical needs in many cancers where the target gene Bcl-2 is overexpressed or where transcription is upregulated. It is known that Bcl-2 is overexpressed in lymphoma, prostate, melanoma, and breast cancers. PNT2258 showed anti-tumor activity against almost all of these indications in mouse models of cancer alone, as well as in combination with rituxamib or docetaxel (FIG. 1). In combination, PNT2258 demonstrated tumor-free survival in all the models.

PNT2258 is cholesterol-rich and negatively-charged. This is unique among lipid delivery systems or polymeric vesicles and contributes to cellular uptake. PNT2258 has shown long circulating half-life, stability, and remarkable antitumor efficacy in animal models. It is also well established that rapidly dividing cells scavenge cholesterol from the circulation/intracellular milieu and cholesterol-rich particles are attracted to the extracellular matrix. Not to be limited by theory, it is postulated that PNT2258 is likely directed into cells through these mechanisms.

PNT2258 reduces Bcl-2 expression and has antitumor efficacy against at least 4 tumor xenograft models. Data suggests that PNT2258 has remarkable synergistic activity in combination with Rituxan® (rituximab) in a Rituxan-resistant xenograft model of NHL and in combination with Taxotere® (docetaxel) in a highly refractory melanoma model. PNT2258's mode of action appears to be multi-factorial, and includes effects on gene expression (gene silencing), apoptosis (cell death) induction as well as stimulation of immune responses to harness the body's innate killing response. These results demonstrate striking therapeutic synergy. Other agents, such as dacarbazine, Vemurafenib (PLX4032), or ipilimumab may also demonstrate therapeutic synergy or an additive effect given with PNT2258.

2. Other Liposomal Delivery Vehicles

Liposomes include, without limitation, cardiolipin based cationic liposomes (e.g., NeoPhectin, available from NeoPharm, Forest Lake, Ill.) and pH sensitive liposomes.

In some embodiments of the present invention, NeoPhectin is utilized as the liposomal delivery vehicle. In some embodiments, the NeoPhectin is formulated with the oligonucleotide so as to reduce free NeoPhectin. In other embodiments, NeoPhectin is present at a charge ratio 6:1 or less (e.g., 5:1, and 4:1) of NeoPhectin to oligonucleotide.

In yet other embodiments, lipids, particularly phospholipids that comprise some liposomes, are conjugated to polyethylene glycol or a derivative thereof, to increase the time that the liposomes circulate in the blood after intravenous injection. (See e.g., Moghimi, S. M. and Szebeni, J, Prog. Lipid Res., 42:463-78, 2003 and Li, W., et al., J. Gene Med., 7:67-79, 2005, which are incorporated herein by reference.) Such liposomes, termed “stealth liposomes” are able to avoid the reticuloentothelial system (RES), resulting in half lives of more than 24 hours in some cases. In one embodiment, the phospholipids in liposomes are conjugated to polyethylene glycol-diorthoester molecules, as described in Li, W., et al., J. Gene Med., 7:67-79, 2005. In other embodiments, the PEG-liposomes are targeted to specific cell receptors. For example, haloperidol conjugated at the distal end of a PEG-linked phospholipids in a cationic liposome targeted sigma receptors that are overexpressed on some cancer cells as described in Mukherjee, et al., J. Biol. Chem., 280, 15619-27, 2005, which is incorporated herein by reference. Anisamide conjugated to PEG-linked phospholipids in liposomes also targets the sigma receptor. (Banerjee, et al., Int. J. Cancer, 112, 693-700, 2004, which is incorporated herein by reference.)

Other liposomic delivery vehicles include lipid nanoparticles which are designed to encapsulate and deliver small oligonucleotides. Examples of lipid nanoparticles include, but are not limited to, for example, stable nucleic-acid-lipid particles (SNALPS; see e.g., Semple et al. Nature Biotech. Lett. (Jan. 17, 2010 doi:10.1038/nbt.1602); and lipidoids (see e.g., Love et al., P.N.A.S. (USA)107(5) 1864-1869).

3. Polymeric Vesicles

In further embodiments, oligonucleotides are sequestered in polymer vesicles. Polymer vesicles can be made from a number of different materials, but in general are formed from block copolymers, for example, polystyrene₄₀-poly(isocyano-L-alanine-L-alanine)ₘ. (See for example, Discher, et al., Science, 297:967-73, 2002; Torchilin, Cell. Mol. Life Sci, 61:2549-59, 2004; Taubert, et al., Curr Opin Chem Biol, 8:598-603, 2004; Lee, et al., Pharm. Res., 22:1-10, 2005; and Gaucher, et al., J. Control. Rel, 109:169-88, 2005, each of which is incorporated herein by reference.) Copolymer vesicles are formed from a number of molecules, including, without limitation, polyacrylic acid-polystyrene, nonionic polyethyleneoxide-polybutadiene, the triblock (polyethyleneoxide)₅-(poly[propyleneoxide])₆₈-(polyethyleneoxide)₅, polyethyleneoxide-poly(propylenesulfide), polyethyleneoxide-polylactide, and polyethylene glycol-polylysine. Many copolymers, particularly those of either amphiphilic or oppositely charged copolymers, including polystyrene₄₀-poly(isocyano-L-alanine-L-alanine)ₘ, self assemble into vesicles in aqueous conditions.

Oligonucleotides can be loaded into the polymer vesicles using several methods. First, the block copolymer can be dissolved along with the oligonucleotides in an aqueous solvent. This method works well with moderately hydrophobic copolymers. Second, for amphiphilic copolymers that are not readily soluble in water, and where a solvent that solubilizes both the oligonucleotides and the copolymer is available, the oligonucleotide and copolymer are dissolved in the solvent and the mixture is dialyzed against water. A third method involves dissolving both the oligonucleotides and copolymer in a water/tert-butanol mixture and subsequent lyophilization of the solvents. The oligonucleotide-loaded vesicles are formed spontaneously when the lyophilized oligonucleotide-copolymer is reconstituted in an injectable vehicle. (Dufresne, et al., in Gurny, (ed.), B. T. Gattefosse, vol. 96, Gattefosse, Saint-Priest, p. 87-102, 2003, which is incorporated herein by reference.)

Polymer vesicles can be targeted to specific cells by tethering a ligand to the outer shell of vesicles by post modification of a copolymer with a bifunctional spacer molecule or by the direct synthesis of heterobifunctional block copolymers.

In yet another embodiment, oligonucleotides can be sequestered in hybrid liposome-copolymer vesicles, as described in Ruysschaert, et. al., J. Am. Chem. Soc., 127, 6242-47, 2005, which is incorporated herein by reference. For example, an amphiphilic triblock copolymers, including poly(2-methyloxazoline)-block-poly(dimethylsiloxan)-block-poly(2-methyloxazoline) can interact with lipids, including phospholipids to form hybrid liposome-copolymer vesicles.

4. Oligonucleotide Modifications

In some embodiments, nucleic acids for delivery are compacted to aid in their uptake (See e.g., U.S. Pat. Nos. 6,008,366, 6,383,811 herein incorporated by reference). In some embodiments, compacted nucleic acids are targeted to a particular cell type (e.g., cancer cell) via a target cell binding moiety (see e.g., U.S. Pat. Nos. 5,844,107, 6,077,835, each of which is herein incorporated by reference).

In some embodiments, oligonucleotides are conjugated to other compounds to aid in their delivery. For example, in some embodiments, nucleic acids are conjugated to polyethylene glycol to aid in delivery (see e.g., U.S. Pat. Nos. 6,177,274, 6,287,591, 6,447,752, 6,447,753, and 6,440,743, each of which is herein incorporated by reference). In yet other embodiments, oligonucleotides are conjugated to protected graft copolymers, which are chargeable drug nano-carriers (PharmaIn), described in U.S. Pat. No. 7,138,105, and U.S. publication numbers 2006/093660 and 2006/0239924, which are incorporated herein by reference. In still further embodiments, the transport of oligonucleotides into cells is facilitated by conjugation to vitamins (Endocyte, Inc, West Lafayette, Ind.; See e.g., U.S. Pat. Nos. 5,108,921, 5,416,016, 5,635,382, 6,291,673 and WO 02/085908; each of which is herein incorporated by reference). In other embodiments, oligonucleotides are conjugated to nanoparticles (e.g., NanoMed Pharmaceuticals; Kalamazoo, Mich.).

In still other embodiments, oligonucleotides are associated with dendrimers. Dendrimers are synthetic macromolecules with highly branched molecular structures. Representative dendrimeric structures are cationic polymers such as starburst polyamidoamine (PAMAM), one of which, SuperFect®, is available from Qiagen (Valencia, Calif.). Other dendrimers include polyester dentrimers described by Gillies, et al., Mol. Pharm., 2:129-38, 2005, which is incorporated herein by reference; phenylacetylene dendrimers, described in Janssen and Meijer, eds, Synthesis of Polymers, Materials science and technology series, Weinheim, Germany: Wiley-VCH Verlag GMBH, Chapter 12, 1999, which is incorporated herein by reference; poly(L-lysine) dendrimer-block-poly(ethylene glycol)-block-poly(L-lysine) dendrimers described by Chol, et al., J. Am. Chem. Soc. 122, 474-80, 2000, which is incorporated herein by reference; amphiphilic dendrimers, described by Joester, et al., Angew Chem Int. Ed. Engl., 42:1486-90, 2003, which is incorporated herein by reference; polyethylene glycol star like conjugates, described by Liu et al., Polym Chem, 37:3492-3503, 1999, which is incorporated herein by reference; cationic phosphorus-containing dendrimers described by Loup, et al., Chem Eur J, 5:3644-50, 1999, which is incorporated herein by reference; poly(L-lysine) dendrimers, described by Ohasaki, et al., Bioconjug Chem, 13:510-17, 2002, which is incorporated herein by reference and amphipathic asymmetric dendrimers, described by Shah, et al., Int. J. Pharm, 208:41-48, 2000, which is incorporated herein by reference. Poly propylene imine dendrimers, described in Tack, et al., J. Drug Target, 14:69-86, 2006, which is incorporated herein by reference; and other dendrimers described above, can be chemically modified to reduce toxicity, for example, as described in Tack, et al.

Dendrimers complex with nucleic acids as do other cationic polymers with high charge density. In general, the dendrimer-nucleic acid interaction is based on electrostatic interactions. Dendrimers can be conjugated with other molecules, such as cyclodextrins to increase efficiency of systemic delivery of dendrimer-nucleic acid complexes. (See Dufes, et al., Adv. Drug Del. Rev, 57, 2177-2202, 2005, and Svenson and Tomalia, Adv. Drug Del. Rev., 57, 2106-29, 2005, both of which are incorporated herein by reference.) Some dendrimers have a flexible open structure that can capture small molecules in their interior, and others have an inaccessible interior. (See Svenson and Tomalia, Adv. Drug Del. Rev., 57, 2106-29, 2005.)

In still further embodiments, oligonucleotides are complexed with additional polymers to aid in delivery (see e.g., U.S. Pat. Nos. 6,379,966, 6,339,067, 5,744,335; each of which is herein incorporated by reference. For example, polymers of N-2-hydroxypropyl methylacrylamide are described in U.S. patent publication number 2006/0014695, which is incorporated herein by reference. Similar cationic polymers are described in International Patent Publication number WO 03/066054 and U.S. patent publication number 2006/0051315, both of which are incorporated herein by reference. Other polymers are described by Intradigm Corp., Rockville, Md.).

5. Other Delivery Methods

In still further embodiments, the controlled high pressure delivery system developed by Mirus (Madison, Wis.) is utilized for delivery of oligonucleotides. The delivery system is described in U.S. Pat. No. 6,379,966, which is incorporated herein by reference.

B. Formulations, Administration and Uses

The compositions of the present invention may be administered orally, parenterally, by inhalation spray, topically, rectally, nasally, intraocularly, buccally, vaginally, or via an implanted reservoir. The term “parenteral” as used herein includes subcutaneous, intravenous, intramuscular, intra-articular, intra-synovial, intrasternal, intrathecal, intraperitoneal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, isotonic sodium chloride solution, and dextrose solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In embodiments where oligomers are prepared in liposomes, the oligomer/liposome formulations may lyophilized or spray-dried for storage. Suitable cryoprotectants and spray-drying protectants may include sugars, for example, but not limited to, glucose, sucrose, trehalose, isomaltose, somaltotriose, mannitol, and lactose. Other cryoprotectants may include dimethylsulfoxide, sorbitol and other agents that alter the glass phase melting temperature (Tₘ). Preparations may include anti-adherents such as magnesium stearate and leucine, buffers, such as Tris or phosphate buffer, and chelating agents, such as EDTA.

The pharmaceutically acceptable compositions of this invention may be orally administered in any orally acceptable dosage form including, but not limited to, capsules, tablets, aqueous suspensions or solutions. In some embodiments, the complex is a mixture of lipids, lipid-like, polymer or polymer-like delivery agents and a cation (e.g. lipids and calcium to form cochleates) or a mixture of lipids lipids, lipid-like, polymer or polymer-like delivery agents and an anion. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In several embodiments, the pharmaceutically acceptable compositions of this invention are formulated for oral administration.

The amount of the compounds of the present invention that may be combined with the carrier materials to produce a composition in a single dosage form will vary depending upon the host treated, the particular mode of administration.

C. Dosing Schedules and Regimen:

In some aspects of the invention, doses of the compositions of the present invention may be administered from 1, 2, 3, 4, 5 or more consecutive or non-consecutive days of a dosing cycle (e.g., 15, 18, 19, 20, 21, 22, 23, 24, 25, 28 or 30 days). In some aspects, doses of the compositions of the present invention may be administered 1, 2, 3, 4, 5 or more days of a dosing cycle (e.g., 15, 18, 19, 20, 21, 22, 23, 24, 25, 28, 30 days), then weekly thereafter.

In some aspects of the present invention, doses of the compositions of the present invention may be administered on a periodic schedule, daily, bidaily, every 2, 3, 4, 5, 6 days, weekly, every 2, 3, 4 weeks, monthly, or more.

Dosing schedules may be administered until certain set points are reached, e.g., based on tumor response measured by RECIST, FDG-PET, or other cancer-based (i.e., lymphoma-based) criteria is or are reached.

In some aspects of the invention, the oligonucleotides of the present invention may be liposome-encapsuled for administration. In some aspects, the composition may be PNT2258.

In some aspects, doses of the liposome-encapsuled oligonucleotides of the present invention may be between about 30 to about 300 mg per m² subject surface area; between about 30 mg per m² subject surface area to about 150 mg/m² (about 30, 40, 50, 60, 70, 80, 90, 100, 110, 120, 130, 140, 150 mg/m².)

In some aspects, doses of the liposome-encapsuled oligonucleotides of the present invention may be administered intravenously; administered intraperitoneally as part of a dialysis regimen to achieve sufficient exposure levels (AUCs).

In some aspects, doses may be administered as an IV infusion of 2 hours to 6 hours; may be administered as a slow IV push of less than 2 hours based on Cₘₐₓ and AUC achieved.

In some aspects, the dose may be administered i.v. at about 0.1, 0.25, 0.5, 1, 1.5, 2.5, 3 hours per dose. In some aspects, medication for treatment tolerability, such as steroids, Benadryl, anti-anxiety (given orally or IV) medication may be administered before or during administration of the compositions of the present invention.

In some aspects, combination therapies useful for treatment of cancer may be administered before, simultaneously or after administration of the compositions of the present invention.

In some aspects, co-medications to alleviate side effects of administration (hydration or prophylactic treatment for potential of tumor lysis syndrome due to action of PNT2258 and/or clearance of BCL-2 sensitive circulation tumor cells in hematological tumors and NHL) may be co-administered, or administered before or after administration of the compositions of the present invention.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents normally administered to treat or prevent a particular disease, or condition, are known as “appropriate for the disease, or condition, being treated.”

In some aspects of the present invention, the dosage cycle comprises a daily dose of the oligomer from 1 mg/m2 to 300 mg/m2 per body surface area of the patient.

In some aspects of the present invention, the daily dose of the oligomer and liposome per surface area of the patient is from about 30 to 150 mg/m².

In some aspects of the present invention, the daily dose of the oligomer and liposome per surface area of the patient together is selected from about 30, 40, 50, 60, 70, 80, 90, 100, 110, 120, 130, 140, or 150 mg/m². In further aspects of the present invention, the daily dose is 20 mg/m².

The other embodiments, the oligomer is administered via an intravenous infusion or intraperitoneally as part of a dialysis regimen to a cancer patient.

In some aspects of the present invention, the infusion or daily dose occurs at a duration between 2 hours and 6 hours or 3 hours or less than 2 hours.

In some aspects of the present invention, the duration is modified based on fixed daily dose or modifying volume of for a fixed daily dose depending on tolerability of a patient. The duration may be decreased or increased to improve tolerability and lessening side effects.

In some aspects of the present invention, the methods further comprising administering a medication for increasing tolerability, wherein the administration of the medication occurs before or during administration of the oligomer of the present invention. These medications for increasing tolerability may include the co-administration of intravenous, subcutaneous, sublingual, oral or rectally administered electrolyte solutions such as dextrose 5% in water or normal saline; co-administration of intravenous, subcutaneous, sublingual, oral or rectally administered corticosteroid; co-administration of intravenous, subcutaneous, sublingual, oral or rectally administered diphenhydramine; co-administration of intravenous, subcutaneous, sublingual, oral or rectally administered anxiolytics; co-administration of intravenous, subcutaneous, sublingual, oral or rectally administered anti-diarrheal medication; co-administration of intravenous, subcutaneous, sublingual, oral or rectally administered supportive care measure such as hematologic growth factor support or erythropoiesis-stimulating agent.

In one aspects of the present invention, the oligomer is SEQ ID NO:1251.

In some aspects of the present invention, the administration of the oligomer is a daily dose of one or more, two or more, three or more, four or more, or five or more days of a dosing cycle.

In other aspects of the present invention, the administration of the oligomer is a daily dose for 1, 2, 3, 4, 5, 6, 7, 8, 9, 10 or more days of a dosing cycle.

In some aspects of the present invention, the dosing cycle is selected from 15, 18, 19, 20, 21, 22, 23, 24, 25, 28, or 30 days.

In some aspects of the present invention, the daily dose is administered on a schedule selected from once or twice per day; every 2, 3, 4, 5, or 6 days; weekly; or every 2, 3, 4 weeks, or monthly.

In some aspects of the present invention, The administration of the oligomer improves overall survival rate or progression-free of the patient.

In other aspects of the present invention, administration produces decreases in tumor size or tumor metabolism of radioloabeled glucose in the patient. The tumor metabolism cab be measured for example by FDG-PET.

In some aspects of the present invention, the administration increases quality of life of a patient, or improvement in ECOG performance and Cheson criteria,

In some aspects of the present invention, The method of any one of claims 1-54, wherein the patient does not experience a clinically significant neutropenia or tumor lysis syndrome.

In some aspects of the present invention, the patient does not experience a clinically significant tumor lysis syndrome after the administration of a hydrating solution, potassium sequestration agent, or allopurinol.

In some aspects of the present invention, the patient experiences a transient decrease in lymphocyte count.

In some aspects of the present invention, the patient experiences a transient decrease in platelet count.

In some aspects of the present invention, the patient does not experience a significant nausea or need for an anti-emetic medication.

In some aspects of the present invention, the patient does not experience a significant diarrhea or need for an anti-diarrheal medication.

In some aspects of the present invention, the administration of the oligomer continues for 1, 2, 3, 4, 5, 6, 7, 8 or more dosing cycles.

### V. Kits

Oligomers of the present invention, including oligomers encapsulated within liposomes of the present invention, may be provided in kits, wherein the kits comprise one or more doses of the liposome-encapsuled oligonucleotides of the present invention may be between about 30 to about 300 mg per m² subject surface area; between about 30 mg per m² subject surface area to about 150 mg/m² (about 30, 40, 50, 60, 70, 80, 90, 100, 110, 120, 130, 140, 150 mg/m².) In some aspects, kits may include one or more doses of additional chemotherapeutic agents or additional oligomers targeting bcl-2 or other genes.

Kits may be designed for home or self-administration by subjects, or in hospitals, in patient, outpatient, or dialysis center etc. settings.

### VI. Examples of Cancer Therapies

The following examples are provided in order to demonstrate and further illustrate certain preferred embodiments and aspects of the present invention and are not to be construed as limiting the scope thereof.

### Example 1

### Liposome Formulations

Various liposome formulations were tested for ease of manufacture and scalability, stability in the presence of serum, and encapsulation efficiency. A series of prototype liposomes having different lipid components, PNT100-to-lipid ratios, and particle size and distribution were evaluated for efficacy and potency against human tumor xenograft models in vivo (see Table 2 below).

TABLE 2
Evaluation of the Lipid Composition of PNT100 Liposome

| Lipid | | | | | | |
|---|---|---|---|---|---|---|
| Components | | | | | | |
| (Molar %) | Prep 1 | Prep 2 | Prep 3 | Prep 4 | Prep 5 | Prep 6 |
| POPC | 30 | 35 | 6 | 6 | 15 | 6 |
| DOPE | | | 24 | 24 | 45 | 24 |
| MOCHOL | | 20 | 47 | 23 | 20 | 47 |
| DOTAP | 10 | | | | | |
| CHEMS | 20 | | | | 20 | 23 |
| Cet-P | | 10 | | | | |
| DMGS | | | 23 | 47 | | |
| CHOL | 40 | 35 | | | | |
| Lipid Categories | CHOL containing | | DMGS containing | | CHEMS containing | |
| | | MOCHOL containing | | | | |
| | | | DOPE containing | | | |
| CF release in full | 8% | 7% | 11% | 12% | 16% | 10% |
| human serum in 4 | | | | | | |
| hours @ 37° C. | | | | | | |
| % Encapsulation | 11 | 53 | 67 | 16 | 60 | 49 |
| Drug/Lipid ratio | 25 | 11 | 26 | 37 | 12 | 20 |
| (μg/μmol) | | | | | | |
| Average diameter | 187/198 | 143/152 | 141/157 | not | 163/158 | 157/174 |
| (nm) and | 0.12/0.09 | 0.16/0.20 | 0.18/0.20 | tested | 0.22/0.36 | 0.23/0.25 |
| Polydispersion | | | | | | |
| index at initial/4 | | | | | | |
| weeks at 5° C. | | | | | | |

Abbreviations:
Prep - Preparation;
CF - carboxyfluorescein;
DOTAP - 1,2-Dioleoy1-3-Trimethylammonium-Propane,
DMGS - dimyristoyl glycerol succinate,
CET-P - Cetyl Phosphate
Particle diameter measured using a Malvern Zetasizer 3000 HSA;
Percent encapsulation is calculated by dividing the drug-to-lipid ratio value of the starting mixture by the value of the final preparation whilst accounting for preparation volumes.

The data suggested that a molar ratio of DOPE/POPC/CHEMS/MOCHOL (24:6:23:47) (i.e., the lipid formulation of PNT2258) provided the optimal balance of reproducibility of preparation, encapsulation efficiency, stability in serum, and efficacy in vivo. This composition responds to pH changes during manufacturing and, it is presumed, also when PNT2258 is administered in vivo. MOCHOL is a pH-titratable lipid that is positively charged at pH 4 during manufacturing, actively binding and thus encapsulating the negatively-charged PNT100 within the liposome interior. When the pH is adjusted to physiological, MOCHOL becomes uncharged and CHEMS becomes negatively charged thus releasing unencapsulated PNT100 from the outer surface of the liposomes. DOPE is believed to act in cooperation with CHEMS as a fusogenic component to destabilize endosomal membranes when PNT2258 is endocytosed in vivo. POPC functions as a structural lipid and, with the cholesterol derivatives CHEMS and MOCHOL, is believed to stabilize the liposomal bilayer as PNT2258 circulates in vivo.

PNT2258 is targeted to be a 2.5 mg/mL solution of PNT100 encapsulated in liposomes and is ready-to-use for IV infusion after thawing. The mixing of an aqueous solution with ethanol is commonly used to encapsulate molecules and enable the formation of liposomes (i.e. ethanolosomes) as the lipids organize to exclude water. PNT100 is highly soluble in aqueous solutions. When PNT100 and the lipids are combined at pH 4, MOCHOL molecules are positively charged and interact with the negatively charged PNT100 to encapsulate it into liposomes. A portion of PNT100 also associates with the outer surface of the liposomes. As the pH is shifted to physiological, any unencapsulated PNT100 is released from the PNT2258 surface because MOCHOL becomes uncharged and CHEMS becomes negatively charged thereby releasing any unencapsulated (free) PNT100 from the surface.

Step 1: Encapsulation of PNT100 into Liposomes

Mixing of PNT100 and lipids to form an ethanolic solution of liposomes.

The purity and the moisture content of PNT100 was corrected for the preparation of the aqueous solution of PNT100 maintained at pH 4. The ethanol solution of lipid wass warned to 55° C. to improve DOPE solubility in ethanol.

The encapsulation of PNT100 into liposomes was evaluated at the two parts: (1) the mixing or loading step where the ratio at which PNT100 and lipids combined and the ethanol content are evaluated and (2) the dilution and pH shift step where the effects ionic strength, pH and ethanol percentage were assessed. The data suggested that PNT100 and lipids can be efficiently combined at ratios of 1:20 to 1:5 (weight per weight, w/w). It was determined that suggests that approximately, 1:8 PNT100-to-lipids in a 30% ethanol followed by the simultaneous dilution to 7.5% ethanol and pH adjustment to 7.4 is optimal. These conditions drove good encapsulation of PNT100, formation of particles of approximately 130 nm mean diameter, and maintain manageable process volumes.

pH shift and Ethanol Dilution

Sodium acetate/acetic acid was chosen to maintain the pH at 4 during mixing because it would allow for proton redistribution between inside and outside of the liposomes after adjustment to physiological pH with the shift buffer. Sucrose was added to maintain osmolality and minimize ionic strength for efficient PNT100-lipid interaction at pH 4. 100 mM sodium chloride, 136 mM sodium monophosphate dibasic pH 9.0 solution was used as the shift buffer to adjust the pH to 7.4 and to increase the ionic strength to maximize the release of non-encapsulated drug.

A high flow continuous process was utilized to ensure rapid mixing times and reduce processing times.

Step 2: Refinement of PNT2258 Particle Diameter and Distribution

The average particle diameter and distribution of PNT2258 during the manufacturing process was monitored by dynamic light scattering. The refinement of particle diameter and distribution by extrusion was implemented to improve the physiochemical and biological properties of PNT2258. This refinement narrowed the particle size distribution of PNT2258 thereby improving filterability for sterile-filtration and consistency of drug-to-lipid ratios. In addition, limited pharmacology data suggest that PNT2258 efficacy was improved and toxicity may be reduced.

The influence of implementing extrusion prior to dilution and the pH shift was also evaluated. The added benefit of extrusion was not observed when performed prior to pH shift.

The pressures used, the flow rates observed, and the number of cycles of extrusion were evaluated to arrive at the appropriate conditions to refine particle size and distribution yet minimize shear forces which would significantly influence PNT100 encapsulation.

Step 3: Ultrafiltration and Diafiltration

Sucrose was used as the dialysis buffer to minimize using additional excipients and is used as a cryoprotectant during PNT2258 freezing and storage.

Step 4: Sterile-Filtration and Fill/Finish

PNT2258 was sterile-filtered using a 0.22 μm sterile-filter, filled into vials and stored frozen until use. Several filter matrices were evaluated including cellulose acetate, polyvinyledine fluoride (PVDF) and polyethersulfone (PES).

Pharmacological testing demonstrated that freezing PNT2258 improved its efficacy. Moreover, repeat free-thaws showed that PNT100 remained encapsulated in PNT2258 and particle diameter and distribution did not change.

### Example 2

### Efficacy of Combination Treatment

The combination of two or more compounds of the present invention provides an inhibition of cancer cell growth that is greater than the additive inhibition of each of the compounds administered separately. For instance, FIG. 1 depicts the results of a study where PNT2258 and the chemotherapeutic agents rituximab or docetaxel were administered alone or in combination to immunosuppressed mice bearing human tumors (i.e. Daudi-Burkitts lymphoma; prostate (PC-3); melanoma (A375); diffuse large cell lymphoma (WSU-DLCL2)). Note that effects of co-administration were in many cases greater than additive; also note that efficacy of PNT2258 was increased with the level of bcl-2 expression in a particular cancer.

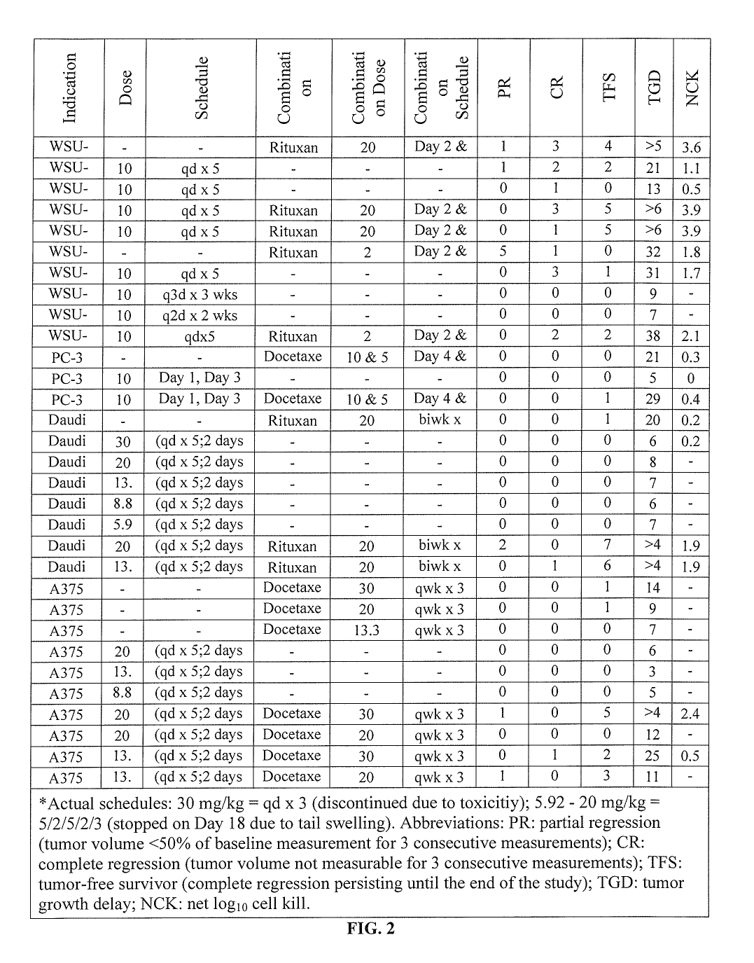

FIG. 2 depicts the percentage of mice with tumors in partial regression (PR) and/or complete regression (CR), as well as the percentage of animals with tumor-free survival (TFS) at the conclusion of the study depicted in FIG. 1.

### Example 3

### Experimental Design of Dose Range Study

The study was an open-label, single-arm, Phase 1 dose-escalation study of PNT2258 in patients with advanced solid tumors. Patients received PNT2258 as an intravenous infusion over 2 hours once daily for 5 consecutive days (Days 1-5) of a 21-day cycle (3 weeks). The initial dose level was 1 mg/m². The dose was doubled until the 64 mg/m² dose level is completed (e.g., Cohort 1=1 mg/m²; Cohort 2=2 mg/m²; Cohort 3=4 mg/m²). Thereafter, dose escalation should proceed with increases of 30 mg/m² increments with the next dose level at 90 mg/m² and continuing to 120 mg/m² and 150 mg/m² in subsequent dose escalations. If a patient dosed at ≦64 mg/m² experienced a ≧Grade 2 toxicity during Cycle 1 (excluding alopecia, nausea or vomiting with less than maximal antiemetic treatment, and diarrhea with less than maximal antidiarrheal treatment), then doses were increased in increments of 33% using cohorts of 3-6 patients guided by the observance of DLTs (dose-limiting toxicities).

DLT on this study were defined as the following treatment-related events experienced during Cycle 1:

Grade 4 neutropenia of greater than 5 days duration, or Grade 3 or greater febrile neutropenia of any duration.

Grade 4 thrombocytopenia.

Any Grade 3 or greater non-hematologic toxicity (except alopecia, nausea/vomiting well-controlled with antiemetics, and laboratory abnormalities felt to be clinically insignificant or that were elevated at baseline).

Any toxicity resulting in a treatment delay beyond 2 weeks.

Acute infusion reaction that requires removal from the study (i.e., does not resolve to baseline or ≦Grade 1 after infusion interruption and resumption at a slower rate).

A 2-Grade increase in AST(SGOT)/ALT(SGPT) for patients with baseline Grade 1 or 2 abnormalities.

The dose at the beginning of each cycle was calculated based on the patient's computed body surface area obtained prior to dosing on Cycle 1 Day 1 unless there was ≧10% change since baseline. If there was a ≧10% change, the current weight was used to calculate the dose for that cycle.

If the patient developed an acute reaction to treatment during infusion, the infusion rate may be reduced according to the investigator's judgment or the infusion may be interrupted until the reaction resolves to baseline or ≦Grade 1; however, total infusion time, including interruptions, may not exceed 6 hours. If toxicities did not resolved to baseline or ≦Grade 1, the infusion was terminated and the patient was removed from the study. Patients experiencing clinically significant infusion reactions received premedication prior to subsequent dosing.

The majority of the patients received PNT2258 as an intravenous infusion over 2 hours once daily for 5 consecutive days (Days 1-5) of a 21-day cycle (3 weeks). However, several patients received PNT2258 at a third (six hours) or half (4 hours) the dose rate either during Cycle 1 or Cycle 2. Further, several patients received PNT2258 for 4 consecutive days rather than 5 consecutive days or several patients received PNT2258 as part of a 28-day cycle (4 weeks). Overall, the dose range of 1-150 mg/m² was well-tolerated. Dose rate and dose schedule were adjusted to patient tolerability and availability to return to the clinic for dosing, thereby providing support for PNT2258 at different dose regimens.

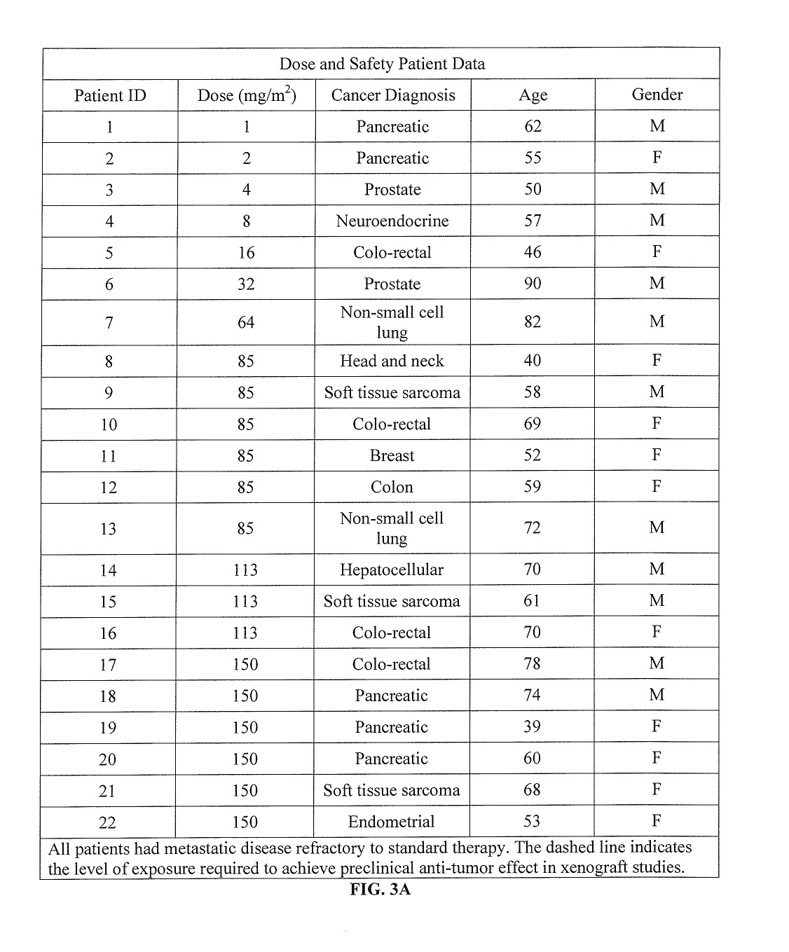

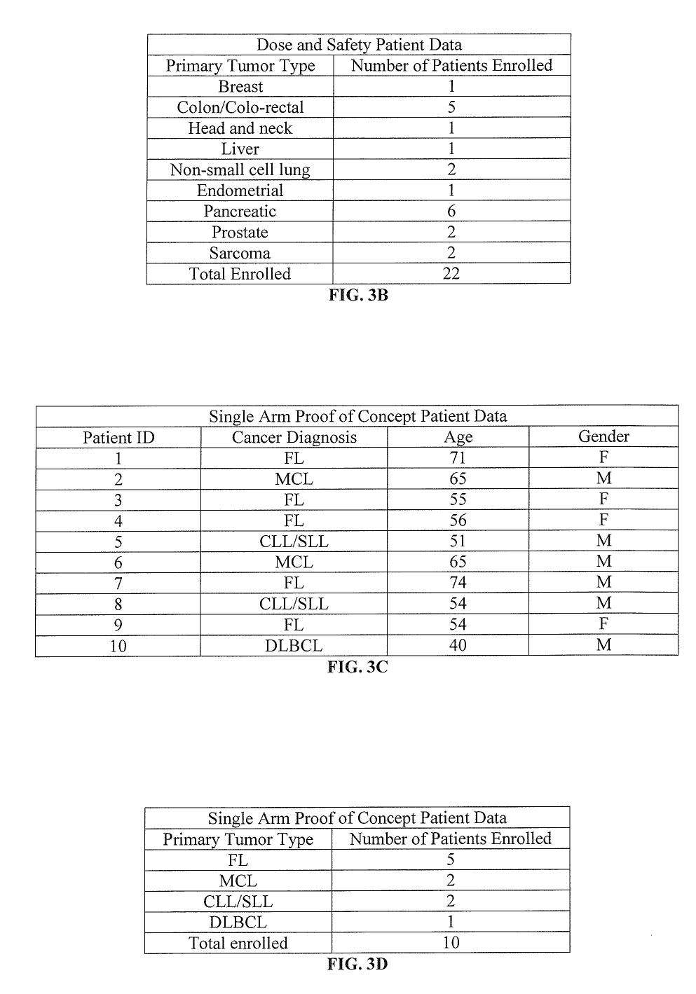

FIG. 3 provides the patient information and assignment into initial dosing regimes for the study, and also shows the number of patients having a particular cancer type.

### Example 4

### Adverse Events

PNT2258 was safely dosed in 22 patients who collectively have received over 60 cycles or the equivalent of over 300 doses. Adverse events are provided below in Table 3.

TABLE 3
Adverse Events
Frequency of All Reported Adverse Events

| | Number | | CTCAE | |
|---|---|---|---|---|
| | of | Frequency | Grade | |
| Adverse Event | Events* | (%) | Range | Attribution |
| Fatigue | 8 | 10.3 | 1-2 | Not Related |
| Infusion | 6 | 7.7 | 1-3 | Related |
| Reaction** | | | | |
| Fever | 4 | 5.1 | 1-2 | Possible |
| Dyspnea | 4 | 5.1 | 1-2 | Not Related |
| Tumor Pain | 4 | 5.1 | 1-3 | Not Related |
| Nausea | 3 | 3.8 | 1-2 | Possible |
| UTI | 3 | 3.8 | 1-4 | Not Related |
| Thrombo- | 4 | 3.8 | 1-3 | Related |
| cytopenia | | | | |

Dose-Limiting Toxicities

| | Number | | | |
|---|---|---|---|---|
| Dose Level | of | | CTCAE | |
| (mg/m²) | Patients | Adverse Event | Grade | Attribution |
| 85 | 1 | Infusion Reaction^{§} | 3 | Related |
| 150 | 1 | Elevated AST/ALT* | 3 | Related |
| 150 | 1 | Decreased Platelets** | 4 | Related |

*A total of 79 adverse events were reported. There were no significant changes in blood pressure, heart rate or changes in EKGs.
**Infusion reaction manifested as back and flank pain.
^{§}Investigators considered this toxicity as “idiosyncratic” in nature. The infusion reaction was manifesting as “flank pain” or “back pain” that resolved after stoppage of the infusion; subsequent patients were given prophylactic dexamethasone. Toxicity was not observed at the highest administered dose.
*Increase in AST/ALT was observed in a patient with metastatic disease to the liver. Elevated levels resolved spontaneously within 48 hours.
**Cycle 2 occurence.
Toxicity observed at the 150 mg/m² dose level defined the maximally-tolerated dose.

Overall, one death (due to progressive disease) and two grade 4 adverse events (sepsis and thrombocytopenia) were reported. The events of death and sepsis were not considered to be related to PNT2258. The principal investigator determined that thrombocytopenia, was related to the PNT2258.

Eight patients experienced a total of ten grade 3 adverse events. The adverse events of renal failure, elevated alkaline phosphatase, uncontrolled pain, pneumonia and urinary tract infection, each reported by one patient, were not considered to be related to the study drug administration. Two patients (dosed at 85 mg/m² and 113 mg/m² respectively) reported grade 3 infusion reactions (four events) that were considered to be related to the study drug. The patients reported the events within minutes of the initiation of study drug administration. The events resolved immediately following the stopping of the infusion.

Fatigue was reported most frequently (10.3%) with eight events reported by seven patients. Seven of the eight events were not related to study drug; one event was possibly related. Three patients experienced a total of five events of infusion reactions (7.7%); all were related to the study drug. Four events each (5.1%) were reported for fever, dyspnea, and tumor pain. One of the four events reported for fever was possibly related to study drug. None of the events reported for dyspnea and tumor pain were related to the study drug. Three events each (3.8%) were reported for nausea, urinary tract infection and thrombocytopenia. The events of nausea and thrombocytopenia were related to the study drug. The events of urinary tract infection were not related to the study drug.

### Example 5

### Pharmacokinetics of PNT2258 in Subjects

PNT2258 pharmacokinetics was determined over the dose range, dose rates and dose schedules administered.

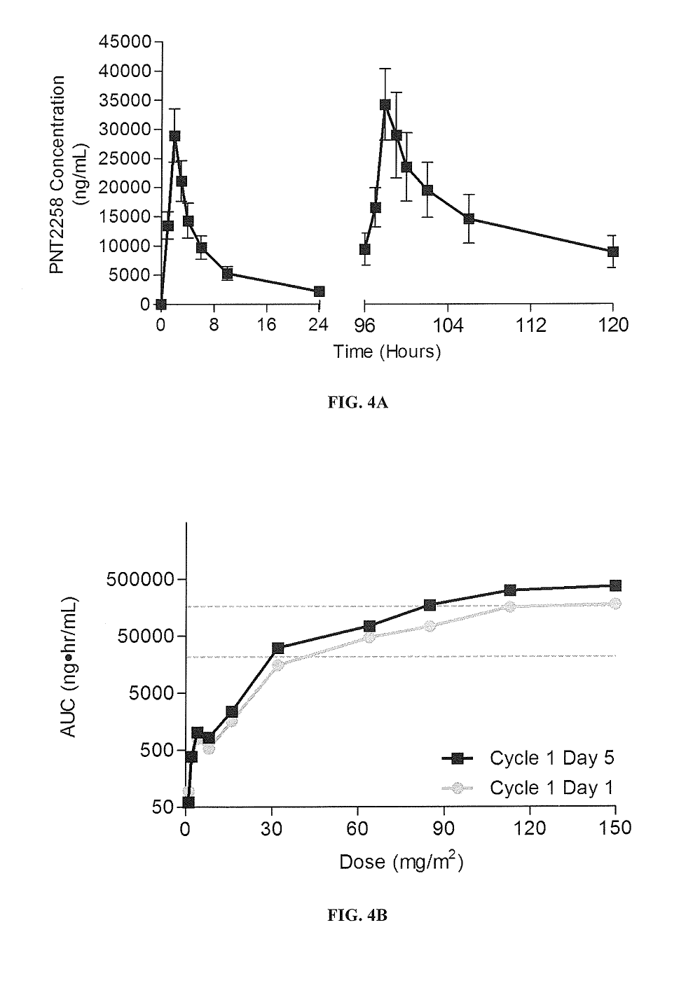

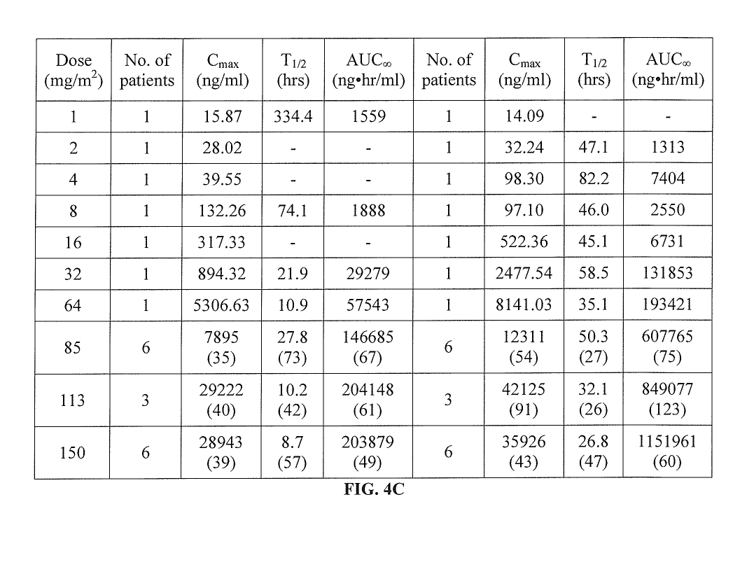

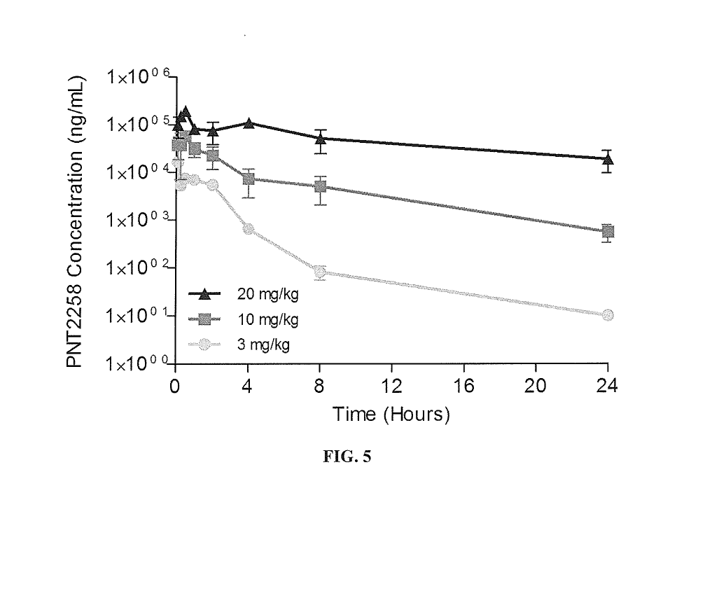

A graph of PNT2258 exposure in a representative cohort (150 mg/m²) in cycle 1, day 1 and cycle 1, day 5 are shown in FIG. 4. The lower panel shows that PNT2258 doses of greater than or equal to 32 mg/m² results in human exposure levels exceeding that required for anti-tumor effect in mouse xenograph models of human tumors (upper and lower threshold levels shown on the graph. The exposure levels in patients compared to mice and are also shown in FIG. 5. The pharmacokinetic assay used for patients is identical to that used for mice. In brief, plasma samples were treated with 10% (v/v) Tween-20 detergent and vortexed prior to analysis to liberate the analyte from the liposome. The samples were then diluted 4-fold with template probe (complementary to the entire sequence of PNT100 using all deoxy nucleotides) containing biotin on its 3′end with a 9-mer overhang to its opposing end. This step was carried out at 37° C. for 1 hour in excess concentrations of template probe (10 nM) to allow for slow and selective binding of intact analyte, minimizing non-specific noise. Following immobilization of the hybridized duplex to a Neutravidin coated plate surface, a signaling probe containing a digoxigenin-label on its 3′-end was added (75 nM). This mixture contained T4 DNA ligase enzyme (2 units/mL) and ATP (0.10 mM) in order to ligate the 3′ terminus of the ODN with the 5′end of the ligation probe. Any un-ligated ligation probe was washed away following a stringent wash step, while any ligation probe that was successfully ligated to the analyte remained intact.

### Example 6

### Tumor Response During Study

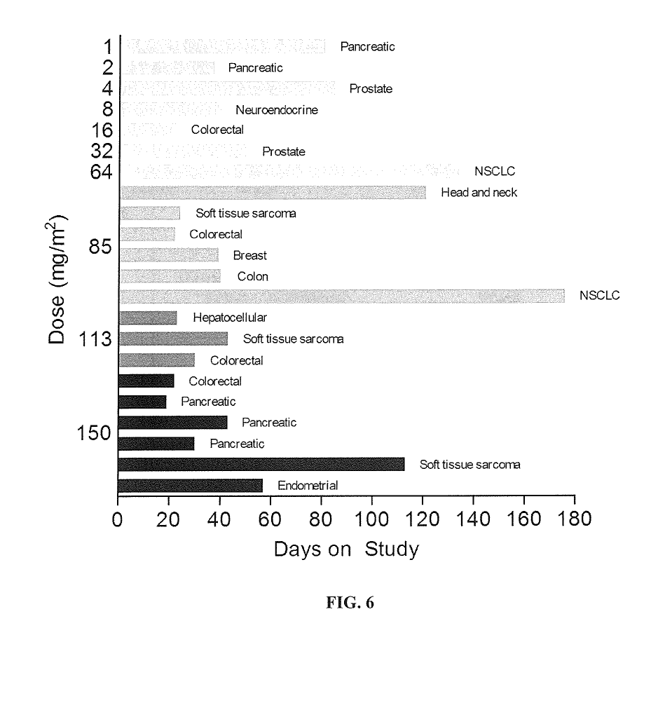

The median number of cycles the subject patients remained in the study is two cycles. The median time a patient remained in the study is 6 weeks. Note that several patients treated with PNT2258 remained in the study for 6-8 cycles (i.e., 16-24 weeks), as shown in FIG. 6. It is interesting to note that the patients who stayed on study longest due to stable disease correspond well with tumor types known to be BCL2-dependent and are in tissues of the reticuloendothelial system (RES).

### Example 6

### Analysis of BCL-2 Expression in Subject Peripheral Blood Mononuclear Cells (PBMCs) Pre- and Post-Dose of PNT2258

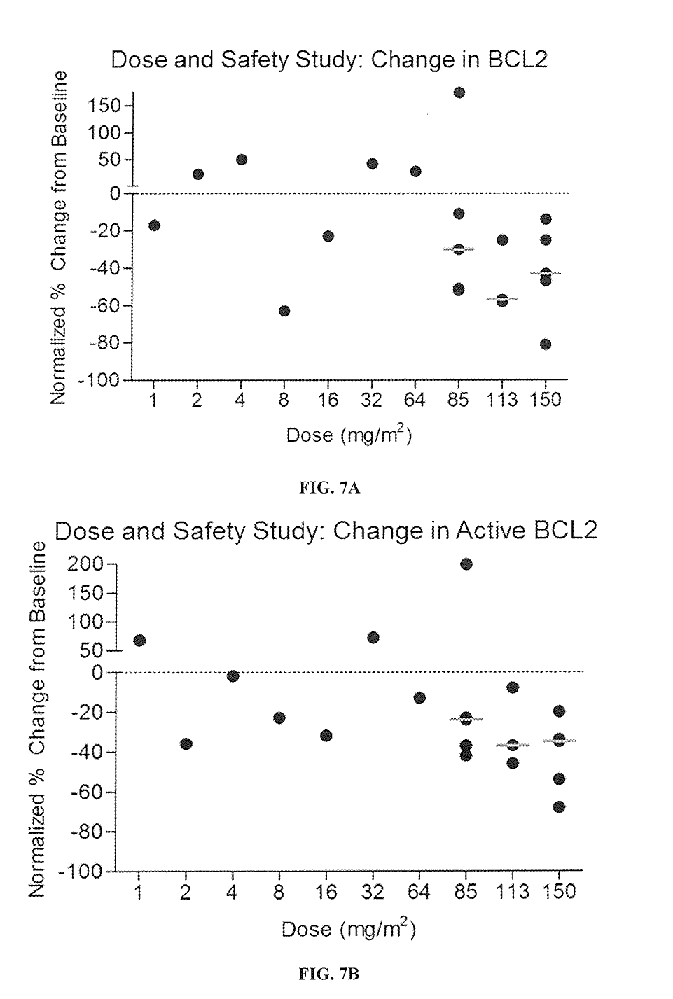

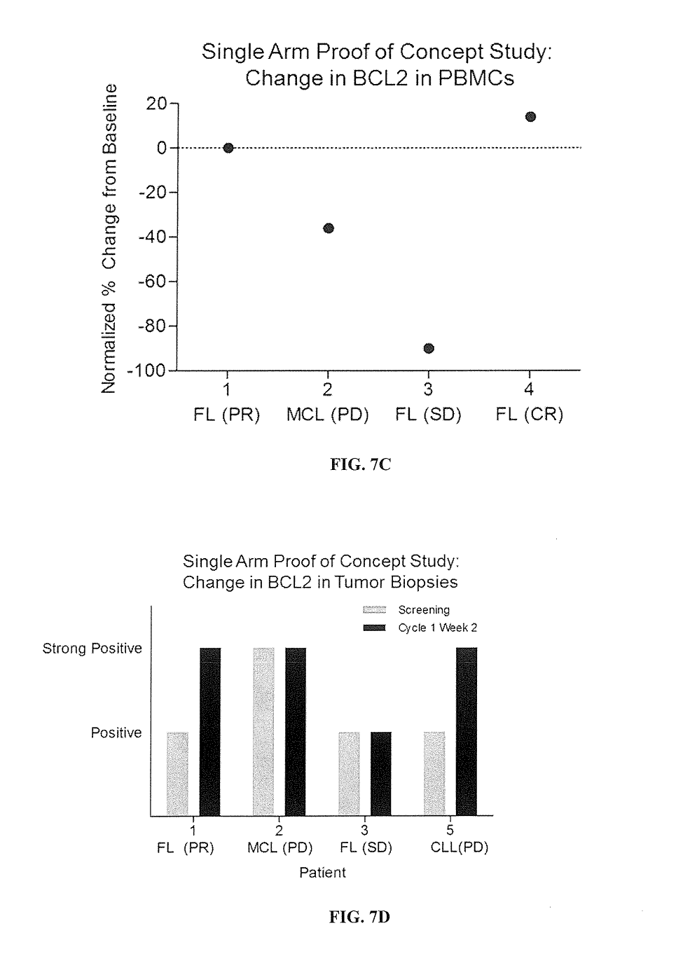

Peripheral blood mononuclear cells (PBMCs) are widely used as surrogates of tumor tissue/cells if the protein of interest is expressed in both the tumor cells and the PBMCs. The percent change in BCL2, activated BCL2, caspase-3 and PARP cleavage from baseline (pre-dose) and post-Day 5 dosing with PNT2258 are shown in FIG. 7 (left). The majority of patients demonstrated a reduction in BCL2 following PNT2258 dosing. Further evidence is provided for a reduction of BCL2 in the observed increase in capsase-3 and PARP cleavage. A reduction in BCL2 initiates a cascade of events leading to the activation of caspase enzymes and the cleavage of PARP, which are hallmarks of apoptotic cell death.

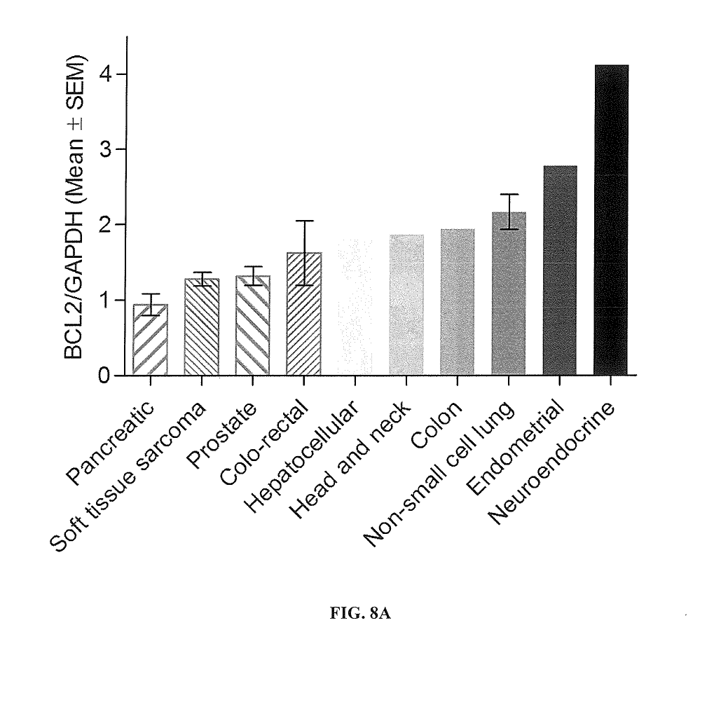

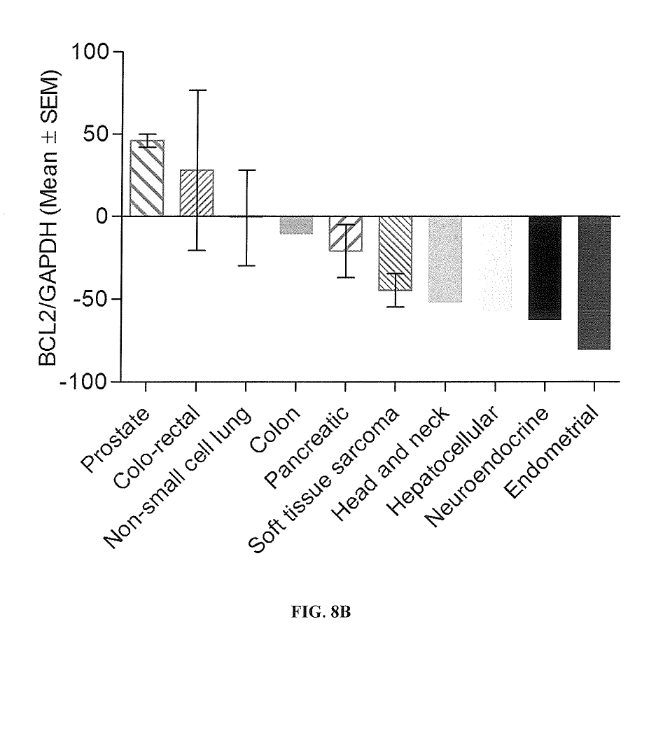

A dose-dependent decrease in BCL2 was noted following PNT2258 treatment with a dose-saturation at approximately 100 mg/m². (FIG. 7, right). Examining the data across subject patient tumor type yields interesting results, where there appears to be differences in the degree of BCL2 reduction with pancreatic, lung and sarcoma cancers showing the largest percentages. (FIG. 8). Of note, prostate and colorectal cancers appear to respond to PNT2258 by increasing BCL2, perhaps in response to treatment.

The extent of BCL2 knockdown in PBMCs is likely an underestimation of the ability of PNT2258 to modulate BCL2 levels. This is due to the fact that PBMCs consist of NK and T cells (lymphocytes, basophils, monocytes, eosinophils) and that this measurement is highly time-dependent. Reductions in lymphocytes, basophils, monocytes are noted following PNT2258 treatment. Therefore, the PBMC population being sampled may be (1) cells that are quiescent and not actively cell cycling or (2) newly released cells. It is further complicated by fact that in cells are likely cleared when BCL2 levels are highly suppressed.

### Example 7

### Analysis of Lymphocytes and Platelet Number/Counts in Patients Dosed with PNT2258

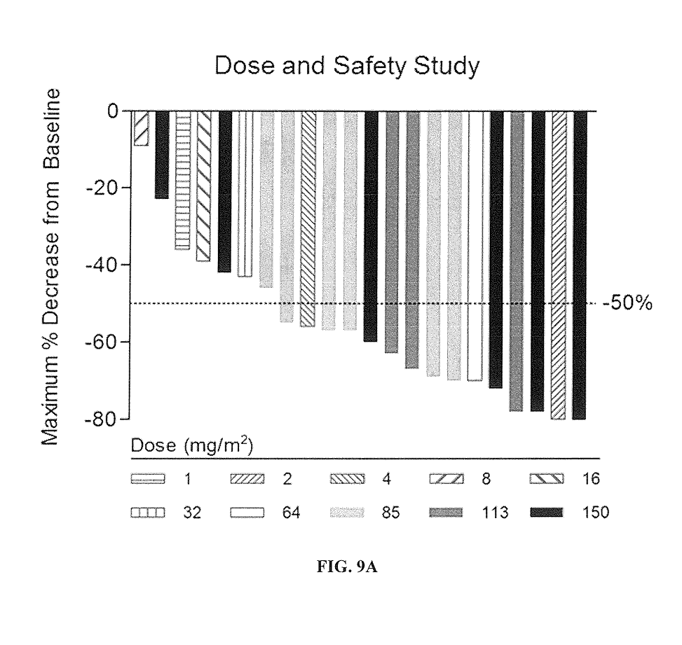

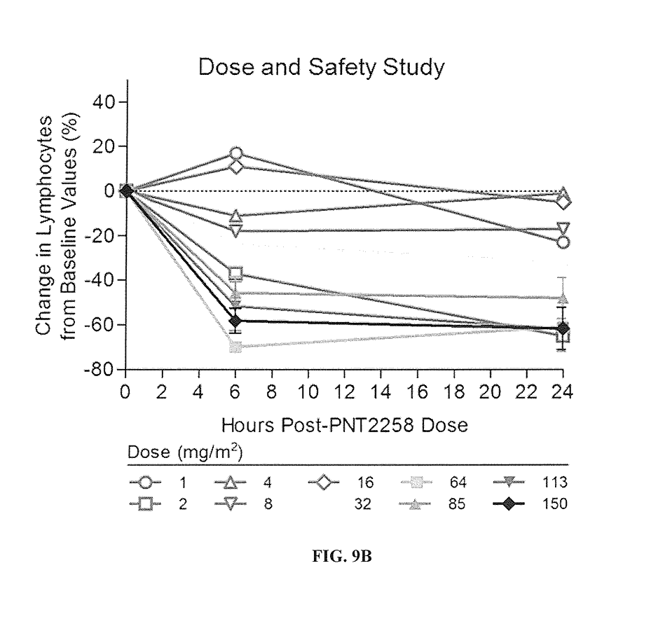

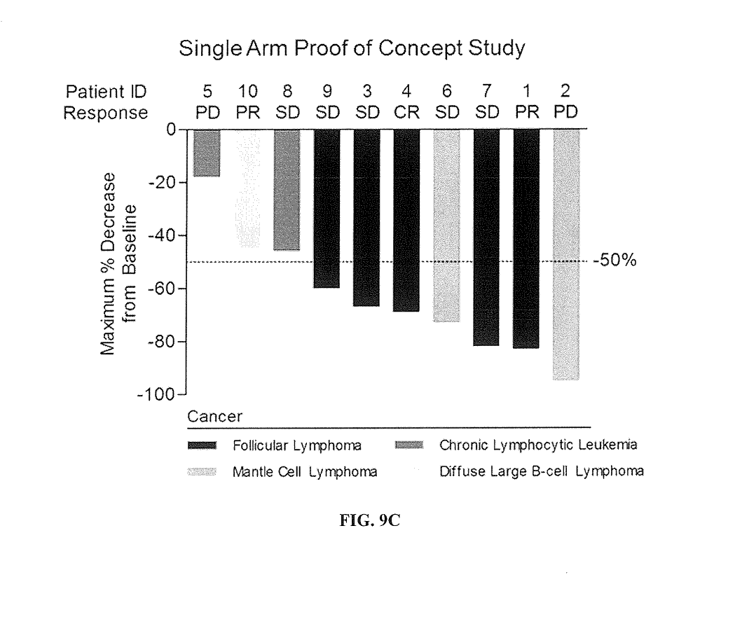

Lymphocytes are intense expressers of BCL2, and their clearance is BCL-2 dependent. BCL2 sequesters Bim, a pro-apototic protein belonging to a distinct subgroup of proteins resembling other BCL2 family members within the short BH3 domain. Bim is essential for hemopoietic cell homeostasis. PNT2258 caused a transient, but clearly measurable decrease in lymphocytes due to targeting of BCL. (FIGS. 9A-C). Lymphocytes decrease during PNT2258 administration, with dose saturation around 100× administration.

Thrombocytopenia is a common side effect of chemotherapeutic agents. For BCL2-targeted agents, platelet reductions can represent a dose-limiting toxicity. This toxicity may result from an on-target effect of modulating BCL2 family members thereby causing enhanced apoptotic clearance of platelets.

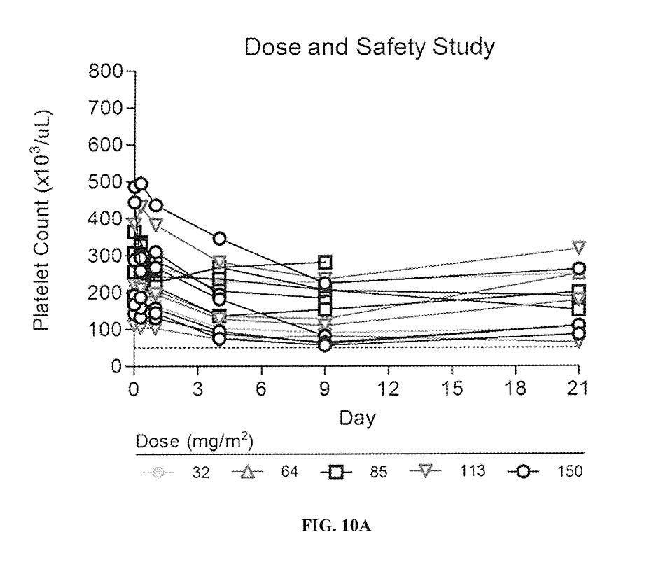

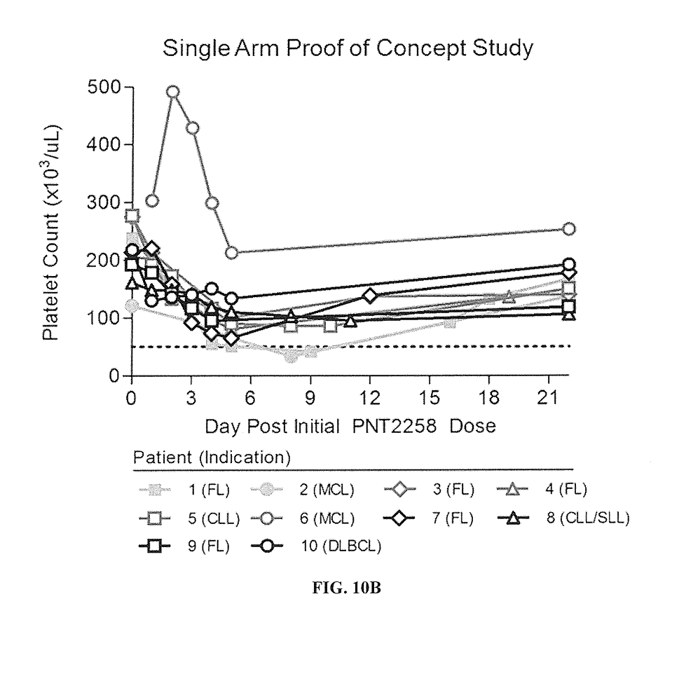

The thrombocytopenia observed with PNT2258 may be a function of BCL2 suppression and a liposome carrier effect on bone marrow and spleen (RES tissues), rather than on circulating platelets. The dose-dependent platelet nadir occurs at days 5-9, suggesting effects that are primarily due to megakaryocytes and on-target bcl-2 effect. The data suggests a downward trend in platelet counts following PNT2258 dosing that began at Cohort 7 with effects observed on Day 5 and nadir on Day 9. (FIGS. 10A-B) The timing of the decrease and the transient effect seen in this study is consistent with the idea that PNT2258 influences megakaryotes rather than circulating platelets.

Platelets are a nuclear and thus should not be influenced by PNT2258. On the other hand, megakaryocytes shed platelets following their maturation. Megakaryocytes are produced primarily by the bone marrow and spleen and tailor their cytoplasm and membranes to enable platelet biogenesis through an enlargement and endomitosis, a process that amplifies DNA by as much as 64-fold. Not to be limited by theory, it is at this point PNT2258 is believed to act, and therefore may influence platelet production and account for the transient and delayed downward trend of platelets noted at higher doses. In contrast, an immediate thrombocytopenia is observed with ABT-263, likely due to its targeted disruption of BCL2, Bcl-xL and Mcl-1 in circulating cells, causing their clearance.

With regards to a carrier effect, the toxicology data in rats and cynomolgus monkeys demonstrate that a reduction in platelet counts were seen only with the high dose of liposome control, PNT2258 and the monkey homologue PNT2258cy, indicative of an overall non-specific effect. Platelet reductions are not observed at lower doses of PNT2258 that are well above the range achieved in the 64 mg/m² cohort. Further, overall, the clinical thrombocytopenia was minimal and could be managed with appropriate treatment. Only one patient experienced Grade 3 then 4 thrombocytopenia.

### Example 8

### Co-Administration of PNT2258 with Metformin

PNT2258 results in cytotoxicity and reduction of BCL-2, in vitro and in vivo animal models, as well as in testing in humans. In humans, an increase in leptin has been seen, hypothesized to be due to PNT2258 downregulation of BCL-2.

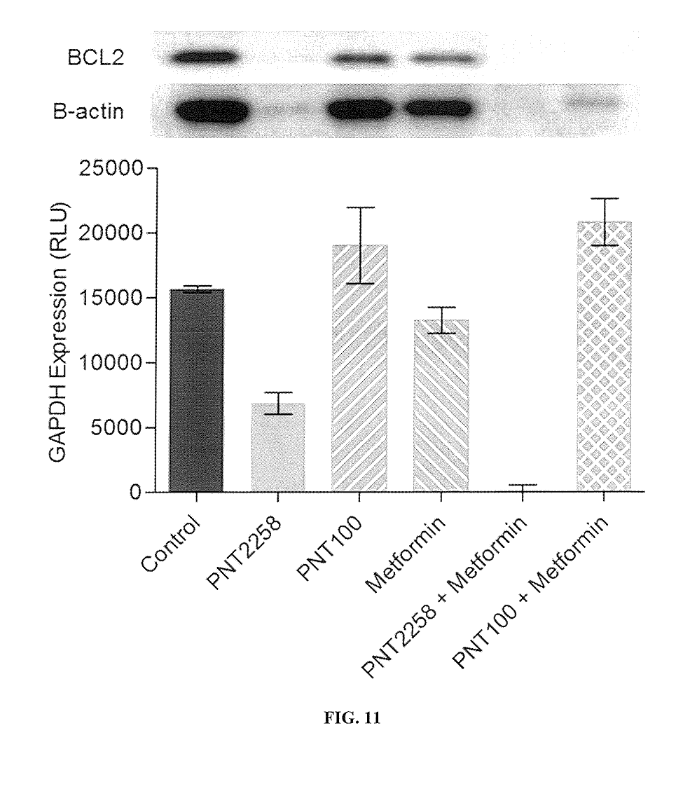

A preliminary study was done to assess whether co-administration of a metabolic-effecting drug, such as the leptin-blocker metformin would have an effect on bcl-2 expression in a Pfeiffer human lymphoma cell line. PNT2258, PNT100, PNT2258+metformin (MTF), PNT100+MTF was administered to the Pfeiffer cells in culture. Bcl-2 expression levels and b-actin levels were monitored by Western blot, as well as the levels of GAPDH in the culture medium. B-actin and GAPDH may be taken as markers of loss of cell function (e.g., after bcl-2 down-regulation—caused apoptosis initiation.) After 6 days in culture, PNT2258+metformin or PNT100+metformin results in synergy for BCL-2, and b-actin. A synergistic reduction of GAPDH was seen with the PNT2258+MTF treatment. (See FIG. 11.) These reductions support the hypothesis that blocking leptin prevents the resistance pathway of PNT2258.

The recently completed clinical trial provides proof-of concept that DNAi agents have promise as a novel class of anticancer therapeutic. PNT2258: (1) demonstrated safety and tolerability at doses of up to 150 mg/m² in patients with advanced solid tumors which represents therapeutic exposures at least five-fold above levels where antitumor effects were observed in preclinical studies, (2) resulted in BCL2 protein reduction with a corresponding increase in caspase-3 and PARP levels in peripheral blood mononuclear cells

What is claimed is:

1. A method of treating cancer, comprising:
administering to a patient an effective amount of an oligonucleotide compound comprising an oligomer that hybridizes under physiological conditions to an oligonucleotide sequence selected from SEQ ID NO:1249 or SEQ ID NO:1254 or the complements thereof, wherein the oligonucleotide is administered on one or more days of a dosing cycle.

2. The method of claim 1 wherein the oligomer is administered in a liposome formulation.

3. The method of claim 2, wherein the liposome formulation is an amphoteric liposome formulation.

4. The method of claim 3, wherein the amphoteric liposome formulation comprises one or more amphoteric lipids.

5. The method of claim 4, wherein the amphoteric liposome formulation is formed from a lipid phase comprising a mixture of lipid components with amphoteric properties.

6. The method of claim 5 wherein the mixture of lipid components are selected from the group consisting of (i) a stable cationic lipid and a chargeable anionic lipid, (ii) a chargeable cationic lipid and chargeable anionic lipid and (iii) a stable anionic lipid and a chargeable cationic lipid.

7. The method of claim 6, wherein the lipid components comprise one or more anionic lipids selected from the group consisting of DOGSucc, POGSucc, DMGSucc, DPGSucc, DGSucc, DMPS, DPPS, DOPS, POPS, DMPG, DPPG, DOPG, POPG, DMPA, DPPA, DOPA, POPA, CHEMS and Cet-P.

8. The method of claim 6 or 7, wherein the lipid components comprise one or more cationic lipids selected from the group consisting of DMTAP, DPTAP, DOTAP, DC-Chol, MoChol, HisChol, DPIM, CHIM, DORIE, DDAB, DAC-Chol, TC-Chol, DOTMA, DOGS, (C18)2Gly+ N,N-dioctadecylamido-glycine, CTAP, CPyC, DODAP and DOEPC.

9. The method of any one of claims 5-8, wherein the lipid phase further comprises neutral lipids.

10. The method of claim 9, wherein the neutral lipids are selected from sterols and derivatives thereof, neutral phospholipids, and combinations thereof.

11. The method of claim 10, wherein the neutral phospholipids are phosphatidylcholines, sphingomyelins, phosphoethanolamines, or mixtures thereof.

12. The method of claim 11, wherein the phosphatidylcholines are selected from the group consisting of POPC, OPPC, natural or hydrogenated soy bean PC, natural or hydrogenated egg PC, DMPC, DPPC or DOPC and derivatives thereof and the phosphatidylethanolamines are selected from the group consisting of DOPE, DMPE, DPPE and derivatives thereof.

13. The method of claim 12, wherein the amphoteric liposomes comprise DOPE, POPC, CHEMS and MoChol.

14. The method of claim 13, wherein the molar ratio of POPC/DOPE/MoChol/CHEMS is about 6/24/47/23.

15. The method of any one of claims 1-14 wherein the oligomer hybridizes under physiological conditions to the oligonucleotide sequence SEQ ID NO:1249 or the complement thereof.

16. The method of claim 15 wherein the oligomer comprises an oligomer selected from the group consisting of SEQ ID NOs:1250, 1251, 1252, 1253, 1267-1477 or the complements thereof.

17. The method of claim 16 wherein the oligomer comprises an oligomer selected from the group consisting of SEQ ID NOs:1250, 1251, 1289-1358 or the complements thereof.

18. The method of any one of claims 1-17, wherein the oligomer comprises SEQ ID NO:1250 or 1251.

19. The method of claim 18, wherein the oligomer comprises SEQ ID NO:1251.

20. The methods of any one of claims 1-19 further comprising administering an additional chemotherapeutic agent.

21. The method of claim 20, wherein the additional chemotherapeutic agent is administered before, simultaneous with, or after the administration of the oligonucleotide compound of claim 1.

22. The method of any one of claims 1-21, wherein the dosage cycle comprises a daily dose of the oligomer from 1 mg/m² to 300 mg/m² per body surface area of the patient.

23. The method of claim 22, wherein the daily dose of the oligomer and liposome per surface area of the patient is from about 30 to 150 mg/m².

24. The method of claim 23, wherein the daily dose of the oligomer and liposome per surface area of the patient together is selected from about 30, 40, 50, 60, 70, 80, 90, 100, 110, 120, 130, 140, or 150 mg/m².

25. The method of claim 23, wherein the daily dose is 100 or 120 mg/m².

26. The method of any one of claims 1-25, wherein the oligomer is administered via an intravenous infusion to a cancer patient.

27. The method of any one of claims 1-25, wherein the oligomer is administered intraperitoneally as part of a dialysis regimen.

28. The method of claim 26 or 27, wherein the infusion occurs at a duration between 2 hours and 6 hours.

29. The method of claim 28, wherein the duration of 3 hours.

30. The method of claim 28, wherein the infusion is less than two hours.

31. The method of claim 28, wherein the duration is modified based on daily dose or volume of daily dose.

32. The method of claim 28, wherein the duration may be decreased to increase tolerability.

33. The method of any one of claims 1-32, further comprising administering a medication for increasing tolerability, wherein the administration of the medication occurs before or during administration of the oligomer of the present invention.

34. The method of claim 33, wherein the medication for increasing tolerability is the co-administration of intravenous, subcutaneous, sublingual, oral or rectally administered electrolyte solution.

35. The method of claim 34, wherein the solution is dextrose 5% in water or normal saline.

36. The method of claim 33, wherein the medication for increasing tolerability is the co-administration of intravenous, subcutaneous, sublingual, oral or rectally administered corticosteroid.

37. The method of claim 33, wherein the medication for increasing tolerability is the co-administration of intravenous, subcutaneous, sublingual, oral or rectally administered diphenhydramine.

38. The method of claim 33, wherein the medication for increasing tolerability is the co-administration of intravenous, subcutaneous, sublingual, oral or rectally administered anxiolytics.

39. The method of claim 33, wherein the medication for increasing tolerability is the co-administration of intravenous, subcutaneous, sublingual, oral or rectally administered anti-diarrheal medication.

40. The method of claim 33, wherein the medication for increasing tolerability is enhanced by the co-administration of intravenous, subcutaneous, sublingual, oral or rectally administered supportive care measure.

41. The method of claim 40, wherein the supportive care measure is hematologic growth factor support or erythropoiesis-stimulating agent.

42. The method of any one of claims 1-41, wherein the oligomer is SEQ ID NO:1251.

43. The method of any one of claims 1-42, wherein the administration of the oligomer is a daily dose of one or more, two or more, three or more, four or more, or five or more days of a dosing cycle.

44. The method of claim 43, wherein the administration of the oligomer is a daily dose for 1, 2, 3, 4, 5, 6, 7, 8, 9, 10 or more days of a dosing cycle.

45. The method of claim 43, wherein the dosing cycle is selected from 15, 18, 19, 20, 21, 22, 23, 24, 25, 28, or 30 days.

46. The method of claim 43, wherein the daily dose is administered on a schedule selected from once or twice per day; every 2, 3, 4, 5, or 6 days; weekly; or every 2, 3, 4 weeks, or monthly.

47. The method of any one of claims 1-46, wherein an overall survival rate of the patient is improved.

48. The method of any one of claims 1-47, wherein a progression-free of the patient is improved.

49. The method of any one of claims 1-48, wherein a tumor size is decreased in the patient.

50. The method of any one of claims 1-49, wherein a tumor metabolism of radioloabeled glucose is decreased.

51. The method of claim 50, wherein the tumor metabolism is measured by FDG-PET.

52. The method of any one of claims 1-51, wherein a quality of life of a patient is increased.

53. The method of any one of claims 1-52, wherein an ECOG performance of a patient status is improved.

54. The method of any one of claims 1-53, wherein a Cheson criteria of a patient is improved.

55. The method of any one of claims 1-54, wherein the patient does not experience a clinically significant neutropenia.

56. The method of any one of claims 1-55, wherein the patient does not experience a clinically significant tumor lysis syndrome.

57. The method of any one of claims 1-56, wherein the patient does not experience a clinically significant tumor lysis syndrome after the administration of a hydrating solution, potassium sequestration agent, or allopurinol.

58. The method of any one of claims 1-57, wherein the patient experiences a transient decrease in lymphocyte count.

59. The method of any one of claims 1-58, wherein the patient experiences a transient decrease in platelet count.

60. The method of any one of claims 1-59, wherein the patient does not experience a significant nausea or need for an anti-emetic medication.

61. The method of any one of claims 1-60, wherein the patient does not experience a significant diarrhea or need for an anti-diarrheal medication.

62. The method of any of claims 1-61, wherein the administration of the oligomer continues for 1, 2, 3, 4, 5, 6, 7, 8 or more dosing cycles.

63. The method of any one of claims 1-62 further comprising the administration of an additional chemotherapeutic agent, immunotherapeutic agent, radiotherapeutic agent selected from metformin, insulin, 2-deoxyglucose, sulfonylureas, anti-diabetic agents generally, mitochondrial oxidative-phosphorylation uncoupling agents, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), mTOR inhibitors, agents that influence cancer metabolism, antibodies or compositions that bind or block CD38, CD19 and CD20, antibodies that stimulate T-cell mediated killing such as PD-1, phosphatidylinositide 3-kinase inhibitors, inhibitors Bruton's tyrosine kinase or spleen tyrosine kinase.

64. A method of treating cancer comprising: administering to a patient an effective amount of a composition comprising:
an oligomer comprising SEQ ID NO:1251,
and a liposome comprising POPC/DOPE/MoChol/CHEMS in about a 6/24/47/23 molar ratio, wherein
wherein the composition is administered on a dosing cycle selected from 15, 18, 19, 20, 21, 22, 23, 24, 25, 28 or 30 days;
wherein the composition is administered daily for 1, 2, 3, 4, 5 or more days of a dosing cycle;
and wherein the dose is between about 30 and 150 mg/m² body surface of the subject.

65. The method of claim 64, wherein the composition is administered on days 1 and 2 of the dosing cycle.

66. The method of claim 64, wherein the composition is administered in combination with an additional therapeutic agent and administration schedule determined by a pharmacokinetic characteristic of the additional therapeutic agent.

67. The method of claim 64, wherein the composition is administered at a dose or schedule determined by saturation of a reticuloendothelial system.

68. The method of claim 64, wherein the oligomer is administered daily at 120 mg/m², and the composition is administered through intravenous administration on days 1-5 of a 21-day schedule.

69. A method of treating cancer, comprising:
administering to a patient an effective amount of an oligonucleotide compound comprising an oligomer that hybridizes under physiological conditions to an oligonucleotide sequence selected from SEQ ID NO:1249 or SEQ ID NO:1254 or the complements thereof, and
administering to the patient an effective amount of an additional chemotherapeutic agent, immunotherapeutic agent, or radiotherapeutic agent selected from metformin, insulin, 2-deoxyglucose, sulfonylureas, bendamustine, gemcitabine, lenalidomide, aurora A kinase, protease inhibitor, pan-DAC inhibitor, pomalidoide, lenalidomide, cytarabine, fludarabine, CPX-351, cytotoxic agents, anti-diabetic agent, mitochondrial oxidative-phoshorylation uncoupling agent, anti-leptin antibodies, leptin receptor agonists, soluble receptors or therapeutics, anti-adiponectin antibodies, adiponectin receptor agonists or antagonists, anti-insulin antibodies, soluble insulin receptors, insulin receptor antagonists, leptin mutens (i.e., mutant forms), BTK inhibitor, mTOR inhibitors, or agents that influence cancer metabolism, antibodies or compositions that bind or block CD38, CD19, CD30, and CD20, antibodies that stimulate T-cell mediated killing such as PD-1, phosphatidylinositide 3-kinase inhibitors, inhibitors Bruton's tyrosine kinase or spleen tyrosine kinase.

70. The method of claim 69, wherein the additional chemotherapeutic agent is a BTK, BCL2, CD20 or PI3K inhibitor to treat chronic lymphocytic leukemia (CLL).

71. The method of claim 69, wherein the additional chemotherapeutic agent is a BTK, BCL2, CD20 or PI3K inhibitor to treat NHL.

72. The method of claim 69, wherein the additional chemotherapeutic agent is comprised of a CD-20 inhibitor, bendamustine, lenalidomide, PI3K inhibitor, mTOR, aurora A kinase, protease inhibitor or pan-DAC inhibitor to treat follicular lymphoma.

73. The method of claim 69, wherein the additional chemotherapeutic agent is comprised of a CD-20 inhibitor, bendamustine, lenalidomide, gemcitabine, PI3K inhibitor, mTOR, aurora A kinase, protease inhibitor or CD30 inhibitor to treat diffuse large B-cell lymphoma.

74. The method of claim 69, wherein the additional therapeutic agent is a CD-20 inhibitor, PI3K inhibitor, BTK inhibitor, BCL2 inhibitor or bendamustine to treat CLL.

75. The method of claim 69, wherein the additional therapeutic agent is selected from pomalidoide or lenalidomide for multiple myleoma.

76. The method of claim 69, wherein the additional therapeutic agent is selected from cytarabine, fludarabine, CPX-351, PI3K inhibitor, or cytotoxic agents to treat acute myeloid leukemia (AML).

77. The method of claim 69, wherein the additional chemotherapeutic agent is administered before, simultaneous with, or after the administration of the oligonucleotide compound.

78. The method of any of claims 1-70, further comprising administering an additional oligonucleotide.
